(19) United States
(12) Patent Application Publication
(10) Pub. No.: US 2010/0086917 A1
(43) Pub. Date: Apr. 8, 2010

(54) Isolated polynucleotides, nucleic acid constructs, methods and kits for localization of rna and/or polypeptides within living cells

(76) Inventors: Liora Haim, Yavne (IL); Jeffrey E. Gerst, Ness Ziona (IL)

Correspondence Address: MARTIN D. MOYNIHAN d/b/a PRTSI, INC., P.O. BOX 16446, ARLINGTON, VA, 22215, US

(21) Appl. No.: 12/311,351

(22) PCT Filed: Sep. 24, 2007
(86) PCT No.: PCT/IL2007/001175

§ 371 Date: Nov. 24, 2009

Related U.S. Application Data
(60) Provisional application No. 60/847,664, filed on Sep. 28, 2006.

(51) Int. Cl. C12Q 1/68; C07H 21/04; C12N 15/74; C12N 5/071; C12N 5/04; C12N 1/19

(52) U.S. Cl. 435/6; 536/23.1; 435/320.1; 435/325; 435/419; 435/254.2

(57) ABSTRACT

An isolated polynucleotide comprising a first nucleic acid sequence which comprises two functionally compatible recognition sites for a site-specific recombination enzyme and a second nucleic acid sequence encoding a protein binding-RNA sequence is provided. Also provided are nucleic acid constructs, methods and kits for localization of an mRNA and/or a polypeptide encoded by a given gene-of-interest within living cells.

### FIELD AND BACKGROUND OF THE INVENTION

The invention relates to isolated polynucleotides, nucleic acid constructs, methods and kits for detecting the localization of RNAs and/or polypeptides encoded by a gene-of-interest within living cells.

mRNA localization is proving to be an important determinant in protein localization. Thus, local mRNA translation is involved in cell-fate determination, cell polarization, and body plan morphogenesis in eukaryotes (3-8). The localization of mRNA within the cytoplasm depends on transport from the nucleus and typically involves anchoring to, and trafficking via, the cytoskeleton. In addition, targeting to particular cytoplasmic regions involves cis-acting elements [e.g., sequences at the 3′-untranslated region (UTR)] as well as trans-acting elements such as RNA-binding proteins. Thus, identification of the temporal and spatial localization of endogenous mRNAs in living cells may contribute to the understanding of cellular processes occurring during normal cell cycle.

One method of examining the localization of endogenous mRNA is RNA in situ hybridization. In this method labeled probes (e.g., RNA, DNA or oligonucleotide probes) which include sequences complementary to the endogenous mRNA-of-interest are applied to fixed cells or tissues under conditions which enable hybridization and the localization of the mRNA-of-interest is detected by the presence of the bound probes to the cells or tissues. However, since in situ hybridization is performed on fixed cells or tissues it offers a good spatial resolution of the RNA within a cell but is limited in the temporal resolution and is unsuited for determining how quickly, or by what route, the mRNA travels to its destination.

Attempts to identify mRNA localization within living cells utilized various expression constructs which induced the expression of exogenous RNA molecules within the cell. For example, U.S. Pat. No. 6,586,240 to Singer R H et al., and Bertrand, E. et al. (Mol. Cell 2, 437-445, 1998) discloses a two-plasmid system that is transformed into cells and enables visualization of a reporter mRNA molecule under the control of an exogenous promoter and a selected sequence of the 3′-UTR belonging to the gene-of-interest. However, since reporter mRNA expression is driven by an exogenous promoter, which differs greatly from the endogenous promoter in controlling the both timing and degree of expression of the gene-of-interest, both the intracellular levels and localization of the reporter mRNA may differ from the naturally occurring mRNA. In addition, if the plasmids include only selected sequences of the 3′-UTR (which may be insufficient for proper localization of the mRNA encoded by the gene-of-interest), the localization of the exogenously-expressed reporter mRNA will be different from that of the endogenous mRNA. Endogenous mRNAs in living cells have been tracked using the QUAL-FRET probe design (Abe and Kool, 2006, PNAS USA 103:263-268).

Recent PCR-based strategies for gene-tagging in the yeast, via homologous recombination, have led to the creation of yeast deletion libraries (9), as well as GFP- and epitope-tagged expression libraries (10, 11). Thus, Huh et al., (11) generated a construct which includes the GFP coding sequence and a selectable marker for homologous recombination in yeast cells which was used to localize polypeptides encoded by a gene-of-interest within living cells. However, following homologous recombination of such a construct, the selectable marker remains in the cell genome, thus substantially increasing the distance between the coding sequence and the 3′-UTR of the gene-of-interest. As a result, transcription of the sequence encoding the polypeptide-of-interest is no longer under the regulatory control of the endogenous 3′-UTR.

### SUMMARY OF THE INVENTION

According to one aspect of the present invention there is provided an isolated polynucleotide comprising a first nucleic acid sequence which comprises two functionally compatible recognition sites for a site-specific recombination enzyme and a second nucleic acid sequence encoding a protein binding-RNA sequence.

According to another aspect of the present invention there is provided an isolated polynucleotide comprising a first nucleic acid sequence which comprises two functionally compatible recognition sites for a site-specific recombination enzyme, a second nucleic acid sequence encoding a protein binding-RNA sequence and a third nucleic acid sequence encoding a reporter polypeptide.

According to yet another aspect of the present invention there is provided a nucleic acid construct comprising the isolated polynucleotide of the invention.

According to still another aspect of the present invention there is provided a cell transformed with the nucleic acid construct of the invention.

According to an additional aspect of the present invention there is provided a system comprising: (i) a first isolated polynucleotide comprising a first nucleic acid sequence which comprises two functionally compatible recognition sites for a site-specific recombination enzyme and a second nucleic acid sequence encoding a protein binding-RNA sequence; and (ii) a second isolated polynucleotide comprising a first nucleic acid sequence which comprises two functionally compatible recognition sites for a site-specific recombination enzyme and a second nucleic acid sequence encoding a reporter polypeptide.

According to yet an additional aspect of the present invention there is provided a system comprising: (i) a first nucleic acid construct comprising a first nucleic acid sequence which comprises two functionally compatible recognition sites for a site-specific recombination enzyme and a second nucleic acid sequence encoding a protein binding-RNA sequence; and (ii) a second nucleic acid construct comprising a first nucleic acid sequence which comprises two functionally compatible recognition sites for a site-specific recombination enzyme and a second nucleic acid sequence encoding a reporter polypeptide.

According to still an additional aspect of the present invention there is provided a transformed cell having a genome which comprises an exogenous polynucleotide being transcriptionally regulated by endogenous 5′ and 3′-untranslated regions of a gene-of-interest, the exogenous polynucleotide comprising a first nucleic acid sequence which comprises at least one recognition site for a site-specific recombination enzyme, and a second nucleic acid sequence encoding a reporter polypeptide.

According to a further aspect of the present invention there is provided a method of identifying a localization of an RNA encoded by a gene-of-interest within a cell, the method comprising: (a) introducing into the cell the isolated polynucleotide of the invention so as to enable homologous recombination of the isolated polynucleotide between endogenous 5′ and 3′-untranslated regions of the gene-of-interest; and (b) detecting the RNA encoded by the gene-of-interest via the protein binding-RNA sequence; thereby identifying the localization of the RNA encoded by the gene-of-interest within the cell.

According to yet a further aspect of the present invention there is provided a kit for identifying a localization of an RNA encoded by a gene-of-interest within a cell, the kit comprising: (i) the isolated polynucleotide of the invention; and (ii) a pair of oligonucleotides which enable homologous recombination of the isolated polynucleotide between endogenous 5′ and 3′-untranslated regions of the gene-of-interest.

According to still a further aspect of the present invention there is provided a method of identifying a localization of a polypeptide encoded by a gene-of-interest within a cell, the method comprising: (a) introducing into the cell an isolated polynucleotide capable of homologous recombination between endogenous 5′ and 3′-untranslated regions of the gene-of-interest, the isolated polynucleotide comprising a first nucleic acid sequence which comprises two functionally compatible recognition sites for a site-specific recombination enzyme, and a second nucleic acid sequence encoding a reporter polypeptide; and (b) detecting within the cell a presence of the reporter polypeptide; thereby identifying the localization of the polypeptide encoded by the gene-of-interest within the cell.

According to still a further aspect of the present invention there is provided a kit for identifying a localization of a polypeptide encoded by a gene-of-interest within a cell, the kit comprising: (i) an isolated polynucleotide capable of homologous recombination between endogenous 5′ and 3′-untranslated regions of the gene-of-interest, the isolated polynucleotide comprising a first nucleic acid sequence which comprises two functionally compatible recognition sites for a site-specific recombination enzyme, and a second nucleic acid sequence encoding a reporter polypeptide; and (ii) a pair of oligonucleotides which enable homologous recombination of the isolated polynucleotide between the endogenous 5′ and 3′-untranslated regions of the gene-of-interest.

According to still a further aspect of the present invention there is provided a method of identifying a localization of an RNA and/or a polypeptide encoded by a gene-of-interest within a cell, the method comprising: (a) introducing into the cell the isolated polynucleotide of the invention so as to enable homologous recombination of the isolated polynucleotide between endogenous 5′ and 3′-untranslated regions of the gene-of-interest; (b) detecting the RNA encoded by the gene-of-interest via the protein binding-RNA sequence; and/or (c) detecting the reporter polypeptide; thereby identifying the localization of the RNA and/or the polypeptide encoded by the gene-of-interest within the cell.

According to still a further aspect of the present invention there is provided a kit for identifying a localization of an RNA and/or a polypeptide encoded by a gene-of-interest within a cell, the kit comprising: (i) the isolated polynucleotide of the invention; and (ii) a pair of oligonucleotides which enable homologous recombination of the isolated polynucleotide between endogenous 5′ and 3′-untranslated regions of the gene-of-interest.

According to further features in the embodiments of the invention described below, the first and the second nucleic acid sequences are sequentially arranged.

According to still further features in the described embodiments the third nucleic acid sequence is positioned upstream of the first nucleic acid sequence.

According to still further features in the described embodiments the isolated polynucleotide further comprising additional nucleic acid sequences which enable homologous recombination with a gene-of-interest.

According to still further features in the described embodiments the pair of oligonucleotides is selected from the group of oligonucleotide pairs consisting of SEQ ID NOs:1 and 2, 3 and 4, 5 and 6, 7 and 8, 9 and 10, 11 and 12, 13 and 14, 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 106 and 107, 108 and 109, 110 and 111, 112 and 113, 114 and 115, 116 and 117, 118 and 119, 120 and 121, 122 and 123, 124 and 125, 126 and 127, 128 and 129, 130 and 131, 132 and 133, 134 and 135, 136 and 137, 138 and 139, 140 and 141, 142 and 143, 144 and 145, 146 and 147, 148 and 149, 150 and 151, 152 and 153, 154 and 155, 156 and 157, 158 and 159, 160 and 161, 162 and 163, 164 and 165, 166 and 167, 168 and 169 and 170 and 171.

According to still further features in the described embodiments the kit further comprising a reagent for detecting the protein binding-RNA sequence.

According to still further features in the described embodiments the kit further comprising a reagent for detecting the reporter polypeptide.

According to still further features in the described embodiments detecting the RNA encoded by the gene-of-interest is effected by expressing within the cell an exogenous polynucleotide encoding a polypeptide capable of binding the protein binding-RNA sequence.

According to still further features in the described embodiments detecting the RNA encoded by the gene-of-interest is effected by introducing into the cell an exogenous polypeptide capable of binding the protein binding-RNA sequence.

According to still further features in the described embodiments the polypeptide capable of binding the protein binding-RNA sequence is attached to a label.

According to still further features in the described embodiments the pair of oligonucleotides is selected from the group of oligonucleotide pairs consisting of SEQ ID NOs:91 and 2, 93 and 4, 95 and 6, and 97 and 8.

According to still further features in the described embodiments the kit further comprising a reagent for detecting the protein binding-RNA sequence and/or the reporter polypeptide.

According to still further features in the described embodiments the kit further comprising packaging materials packing the isolated polynucleotide and the pair of oligonucleotides.

According to still further features in the described embodiments the kit further comprising at least one reagent for PCR amplification of the isolated polynucleotide with the pair of oligonucleotides.

According to still further features in the described embodiments expression of the polynucleotide is regulated by the endogenous 5′ and 3′-untranslated regions of the gene-of-interest.

According to still further features in the described embodiments the first nucleic acid sequence further comprising a selectable marker.

According to still further features in the described embodiments the two functionally compatible recognition sites are positioned so as to enable excision of the selectable marker following homologous recombination of the isolated polynucleotide in a genome of a cell.

According to still further features in the described embodiments the each of the two functionally compatible recognition sites for the site-specific recombination enzyme comprises a loxP sequence.

According to still further features in the described embodiments the site-specific recombination enzyme comprises a Cre recombinase.

According to still further features in the described embodiments the protein binding-RNA sequence is capable of binding a protein selected from the group consisting of a bacteriophage MS2 coat protein, an IRP1 protein, a zipcode binding protein, a box C/D snoRNA binding protein and an aptamer.

According to still further features in the described embodiments the cell is a living cell.

According to still further features in the described embodiments the cell is a eukaryotic cell.

According to still further features in the described embodiments the cell is a yeast cell.

According to still further features in the described embodiments the reporter polypeptide comprises an antibody binding antigen or a labeled protein.

According to still further features in the described embodiments the RNA encoded by the gene-of-interest is selected from the group consisting of ASH1, SRO7, PEX3, OXA1, PEX14, PEX13, PEX11, PEX15, PEX1, PEX5, AAT2, GPD1, DCI1, POX1, PCS60, MDH3, PCD1, PEX12 and POT1.

According to still further features in the described embodiments the gene-of-interest is selected from the group consisting of a peroxin and a peroxisomal matrix protein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c are schematic representations of exemplary nucleic acid constructs of the present invention. FIG. 1a—A schematic representation of the MS2 loop genomic tagging strategy (m-TAG). (1)—Forward and reverse oligonucleotide primers having identity to the coding region (including stop codon) and 3′-UTR of a given open reading frame (ORF) of a gene-of-interest, respectively, are used to amplify a template cassette by PCR. The template cassette contains 12 MS2 loop sequences (MS2L) and a selectable marker (SpHIS5 in this case) flanked by loxP sites. PCR amplification yields the product shown in step (2). (2)—The PCR product is transformed into yeast and homologous recombination results in integration into the ORF between the coding region and 3′-UTR to yield the allele shown in step (3). (3) cre recombinase expression excises the selectable marker located between the loxP sites, leaving one loxP site and MS2L juxtaposed between the coding region and 3′-UTR, as shown in step (4). (5) After verification of integration and marker excision by PCR analysis and sequencing, cells are transformed with a plasmid expressing MS2-CP-GFP(x3) in order to visualize mRNA localization. FIG. 1b—A schematic representation of the mRFP and MS2 loop genomic tagging strategy. (1)—A forward oligonucleotide primer having identity to the coding region (lacking the stop codon) of a given ORF and the 5′ end of mRFP, and a reverse primer having identity to the 5′ end of the ORF 3′-UTR are used to amplify a template cassette by PCR. The template cassette contains 12 MS2 loop sequences (MS2L) and a selectable marker (SpHIS5 in this case) flanked by loxP sites. PCR amplification yields the product shown in step (2). (2)—The PCR product is transformed into yeast and homologous recombination results in integration into the ORF between the coding region and 3′-UTR to yield the allele shown in step (3). (3) cre recombinase expression excises the selectable marker located between the loxP sites, leaving one loxP site and MS2L juxtaposed between the ORF coding region-mRFP and 3′-UTR, as shown in step (4). (5) After verification of integration and marker excision by PCR analysis and sequencing, cells are transformed with a plasmid expressing MS2-CP-GFP(x3) in order to visualize mRNA localization. Protein localization is visualized by RFP fluorescence. FIG. 1c—A schematic representation of the mRFP genomic tagging strategy. (1)—A forward oligonucleotide primer having identity to the coding region (lacking the stop codon) of a given ORF and the 5′ end of mRFP, and a reverse primer having identity to the 5′ end of the ORF 3′-UTR are used to amplify the template cassette by PCR. The template cassette contains the mRFP1 sequence and a selectable marker (SpHIS5 in this case) flanked by loxP sites. PCR amplification yields the product shown in step (2). (2)—The PCR product is transformed into yeast and homologous recombination results in integration into the ORF between the coding region and 3′-UTR to yield the allele shown in step (3). (3) cre recombinase expression excises the selectable marker located between the loxP sites, leaving one loxP site juxtaposed between the ORF coding region-mRFP and 3′-UTR, as shown in (4). Protein localization is visualized by RFP fluorescence, after verification of integration and marker excision by PCR analysis and sequencing.

FIGS. 2a-d depict PCR analysis and detection of MS2 loop integration and marker excision. FIGS. 2a-b are a schematic presentation (FIG. 2a) and a gel image (FIG. 2b) depicting the verification of loxP::SpHIS5::loxP::MS2L integration into the ASH1 locus. Integration of the loxP::SpHIS5::loxP::MS2L cassette after transformation into wild-type yeast was performed with the reverse oligonucleotide used originally to amplify the insertion cassette (SEQ ID NO:2) and a forward oligonucleotide complimentary to the coding region (5′ to the predicted site of integration at the ASH1 locus) (SEQ ID NO:59). These primers were used to amplify genomic DNA derived from wild-type (WT) control cells (lane 3) or cells transformed with the ASH1::loxP::SpHIS5::loxP::MS2L::ASH1^{3′-UTR} fragment (lane 4). The PCR product obtained from the transformed strain (lane 4) had a mobility ˜2.2 kb in agarose gels, which corresponds to the 12 MS2 loops and SpHIS5 marker. This was verified by DNA sequencing (data not shown). No fragment was amplified from genomic DNA derived from the control cells (lane 3) or from the negative control lacking DNA (No DNA, lane 2). M=DNA mobility markers (lane 1). FIGS. 2c-d are a schematic presentation (FIG. 2c) and a gel image (FIG. 2d) depicting the verification SpHIS5::loxP marker excision and ASH1::loxP::MS2L::ASH1^{3′-UTR} expression. After cre recombinase induction and selection on medium containing histidine, genomic DNA and total RNA was extracted from both wild-type control cells (WT) and the putative ASH1::loxP::MS2L::ASH1^{3′-UTR} integrated strain (ASH1^{INT}). Amplification of genomic DNA (genomic DNA, lanes 2 and 3) was performed using forward and reverse oligonucleotides complimentary to the coding region (5′ to the site of insertion) (SEQ ID NO:59) and 3′-UTR (3′ to the site of insertion) (SEQ ID NO:60), respectively. The mobility of the PCR product obtained from the integrated strain (lane 3) was ˜790 bp larger than that obtained from the wild-type strain (lane 2). No product was obtained from the control reaction lacking DNA (No DNA, lane 8). RT-PCR of total RNA obtained from the integrated strain (lane 7), using the same oligonucleotides, also yielded a fragment ˜790 bp larger than that obtained from total RNA derived from wild-type control cells (lane 6). DNA sequencing demonstrated that the 12 MS2 loops are present in the transcribed mRNA derived from ASH1: :loxP::MS2L-ASH1^{3′-UTR} cells (not shown). PCR performed on total RNA (not reverse transcribed) yielded no products (lanes 4 and 5), revoking DNA contamination. M=DNA mobility markers (lane 1).

FIGS. 3a-f are representative fluorescent microscopy images depicting the localization of endogenous ASH1 mRNA to the bud tip of yeast cells in vivo. Strain cells with the integrated ASH1::loxP::MS2L::ASH1^{3′-UTR} cassette were further transformed with plasmids expressing MS2-CP fused with one GFP molecule (MS2-CP-GFP) (FIG. 3a), two GFP molecules (MS2-CP-GFP(x2) (FIG. 3b), or three GFP molecules (MS2-CP-GFP(x3) (FIGS. 3c-f). Shown are cells at the early G2-M phase (FIGS. 3a-c and e), the S phase (FIG. 3d) and the late G2-M phase (FIG. 3f). GFP granules in the bud mark the location of granular mRNA. All pictures are merged windows of DIC and GFP fluorescence microscopy

FIGS. 4a-c are representative fluorescence microscopy (FIGS. 4b-c) and DIC (FIG. 4a) images depicting endogenous localization of SRO7 mRNA to the bud tip in vivo. SRO7::loxP::MS2L::SRO7^{3′-UTR} integrated cells were transformed with a plasmid expressing MS2-CP-GFP(x3). The GFP granule at the bud tip marks the localization of granular SRO7 mRNA.

FIGS. 5a-l are representative fluorescence (FIGS. 5b-d, f-h, j-l) and DIC (FIGS. 5a, e, i) microscopy images depicting endogenous localization of PEX3 mRNA to the ER in vivo. PEX3::loxP::MS2L::PEX3^{3′-UTR} integrated cells were transformed with plasmids expressing MS2-CP-GFP(x3) and Sec63-RFP (an ER marker). The green fluorescence signal represents granular PEX3 mRNA (FIGS. 5b, f, i), while the red fluorescence signal represents ER staining (FIGS. 5c, g, k). Note the co-localization of the PEX3 mRNA (green fluorescence signal) to the ER (red fluorescence signal).

FIGS. 6a-l are representative fluorescence (FIGS. 6b-d, f-h and j-l) and DIC (FIGS. 6a, e and i) microscopy images depicting endogenous localization of OXA1 mRNA to mitochondria in vivo. OXA1::loxP::MS2L::OXA1^{3′-UTR} integrated cells were transformed with plasmids expressing MS2-CP-GFP(X3) and Oxa1-mRFP (a mitochondrial marker). The green fluorescence signal represents granular OXA1 mRNA, while the red fluorescence signal represents Oxa1-mRFP labeling of mitochondria. Note the co-localization of OXA1 mRNA to the mitochondria.

FIGS. 7a-x are representative light (FIGS. 7a, e, i, m, q, u) fluorescence (FIGS. 7b, c, f, g, j, k, n, o, r, s, v, w) and merged (FIGS. 7d, h, l, p, t, x) microscopy images depicting that endogenous mRNAs encoding peroxins localize mainly to peroxisomes. ORF::loxP::MS2L::3′UTR integrated cells [wherein the open reading frame (ORF) refers to an ORF of the PEX5, PEX15, PEX13, PEX11, PEX14 or PEX1 genes] were transformed with a plasmids expressing MS2-CP fused with three GFP molecules and RFP-PTS1, as a marker for peroxisomes. The cells were grown on SC medium containing oleate (SC, 0.2% Glucose, 0.2% Oleate, 0.25% Tween). The localization of mRNA to peroxisomes is given in percent (%).

FIGS. 8a-t are representative light (FIGS. 8a, e, i, m, q) fluorescence (FIGS. 8b, c, f, g, j, k, n, o, r, s) and merged (FIGS. 8d, h, l, p, t) microscopy images depicting the localization of endogenous mRNAs encoding peroxisomal matrix proteins. ORE::loxP::MS2L::3′UTR integrated cells [wherein the open reading frame (ORF) refers to an ORF of the PCS60, DC11, POX1, GPD1 or AAT2 genes encoding peroxisomal matrix proteins] were transformed with plasmids expressing MS2-CP fused with three GFP molecules and RFP-PTS1, as a marker of peroxisomes. The cells were grown on SC medium containing oleate (SC containing 0.2% Glucose, 0.2% Oleate, and 0.25% Tween). The localization of mRNA to peroxisomes is given in percent (%).

### DESCRIPTION OF THE PREFERRED EMBODIMENTS

The present invention is of a genomic tagging strategy which can be used to localize an RNA (preferably mRNA) and/or a polypeptide encoded by a gene-of-interest within living cells. Specifically, the present invention is of isolated polynucleotides, nucleic acid constructs, cells transformed with the isolated polynucleotides and nucleic acid constructs, methods and kits for localization of RNA and/or polypeptide encoded by a gene-of-interest within living cells.

The principles and operation of the isolated polynucleotides, nucleic acid construct, methods and kits of localizing RNA and/or polypeptide encoded by a gene-of-interest according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

mRNA localization is proving to be an important determinant in protein localization, yet no technique is currently available for examining the localization of endogenous mRNAs in living cells. In situ hybridization can be used to examine endogenous mRNA localization, but can only be performed with fixed cells or tissues. Plasmids can be used to exogenously express mRNAs bearing binding sites for an RNA binding protein (RBP, e.g., the MS2 coat protein), and when co-expressed with the RBP fused with a fluorescent protein (e.g., green fluorescent protein) can localize the mRNAs in vivo [U.S. Pat. No. 6,586,240 to Singer R H et al., and Bertrand, E. et al. (Mol. Cell 2, 437-445, 1998)]. However, as expression of the reporter mRNA is driven by an exogenous promoter, and since the plasmid includes only selected sequences of the 3′-UTR which may be insufficient for proper mRNA localization, the localization of the reporter mRNA may be different from that of the endogenous mRNA encoded by the gene-of-interest.

In order to localize polypeptides encoded by genes-of-interest, Huh et al., (11) generated a construct which includes the GFP coding sequence and a selectable marker for homologous recombination in yeast cells. However, following homologous recombination of such a construct, the selectable marker remains in the cell genome, thus increasing the distance between the coding sequence and the 3′-UTR of the gene-of-interest. As a result, the transcription of the sequence encoding the polypeptide-of-interest is no longer under the regulatory control of the endogenous 3′-UTR. Thus, there is a fundamental need for an in vivo strategy of tagging endogenously expressed mRNAs and/or polypeptides for visualization in living cells.

While reducing the present invention to practice, the present inventors have uncovered a genomic-tagging strategy that allows for the localization of RNAs (preferably mRNA) expressed by a gene-of-interest within living cells [see the Examples section which follows and Haim, L., et al., 2007 (“A PCR-based genomic integration method to visualize the localization of endogenous mRNAs in living yeast.” Nat. Methods 4:409-412) and Tyagi S. 2007 (News and Views, Nat. Methods 4:391-392)]. This strategy is based on tagging the gene-of-interest, while still allowing it to be naturally expressed in living cells under its endogenous transcriptional control. This is in sharp contrast to prior attempts by Singer R H et al. (U.S. Pat. No. 6,586,240) and Bertrand, E. et al. (Mol. Cell 2, 437-445, 1998) who used a plasmid-based system for the exogenous expression of mRNA from a gene-of-interest under the transcriptional control of an exogenous promoter and selected sequences derived from the 3′-UTR of the gene-of-interest.

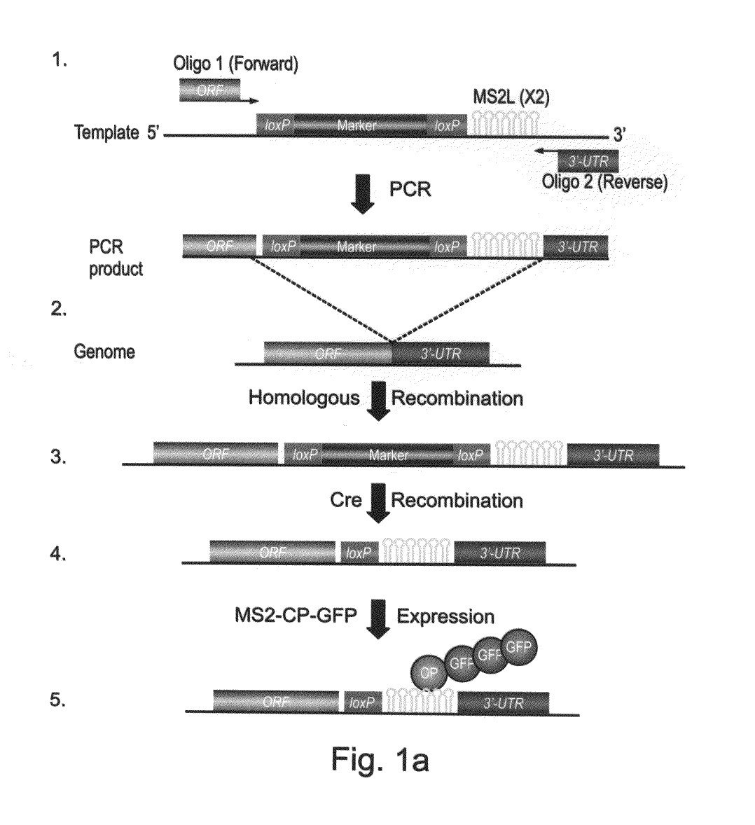

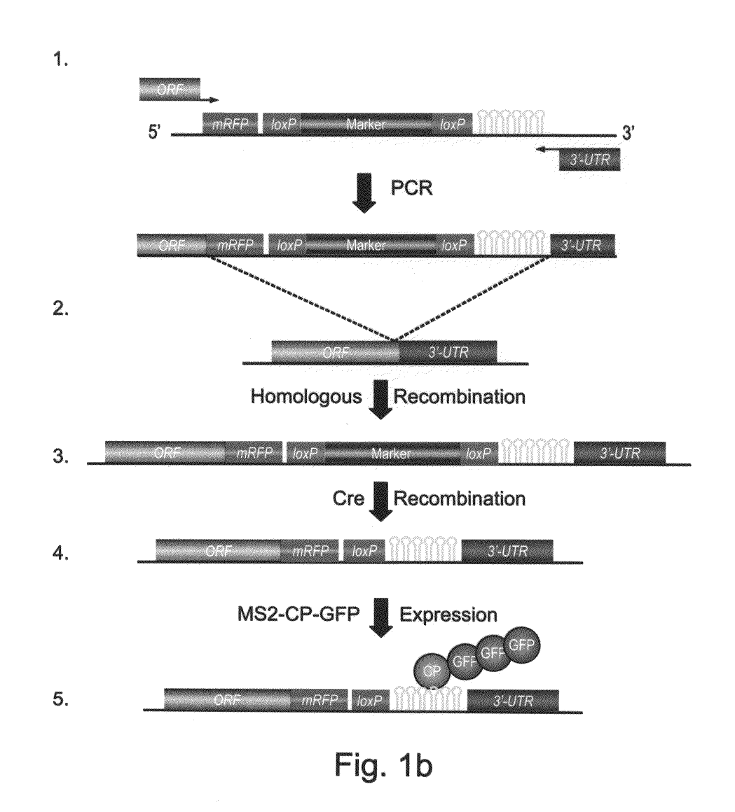

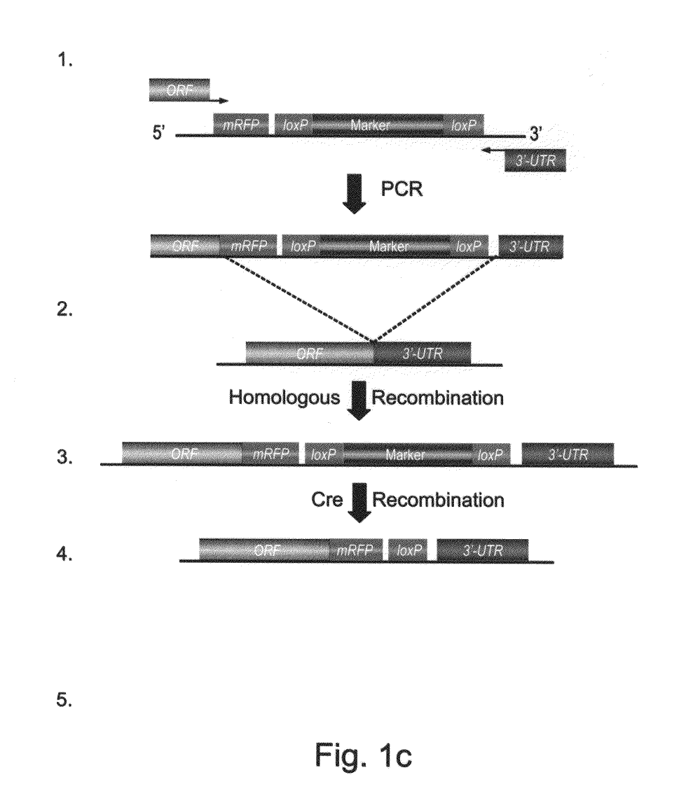

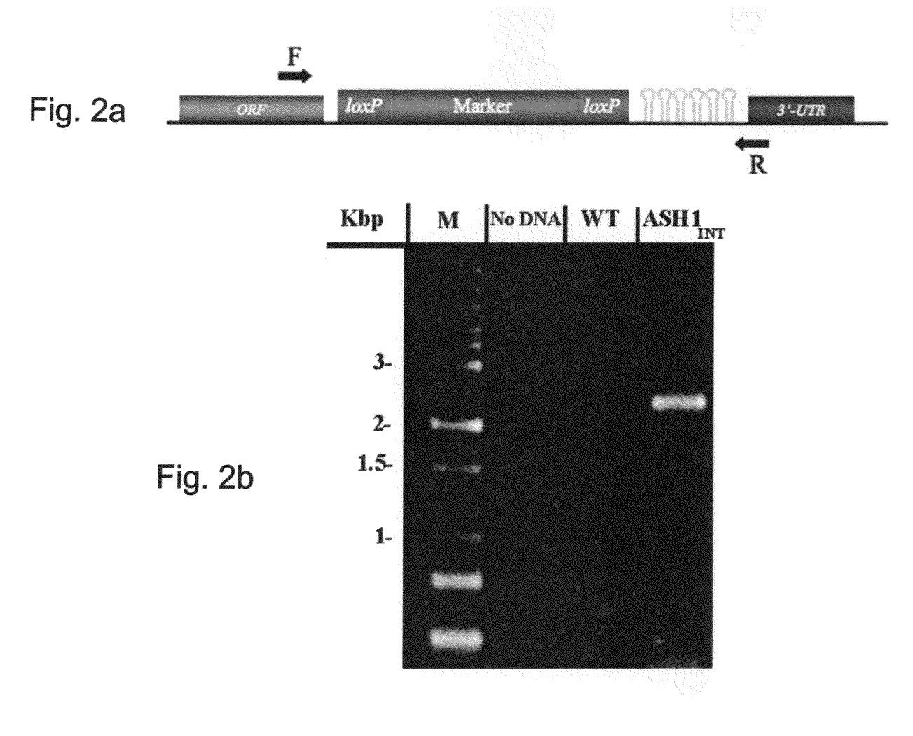

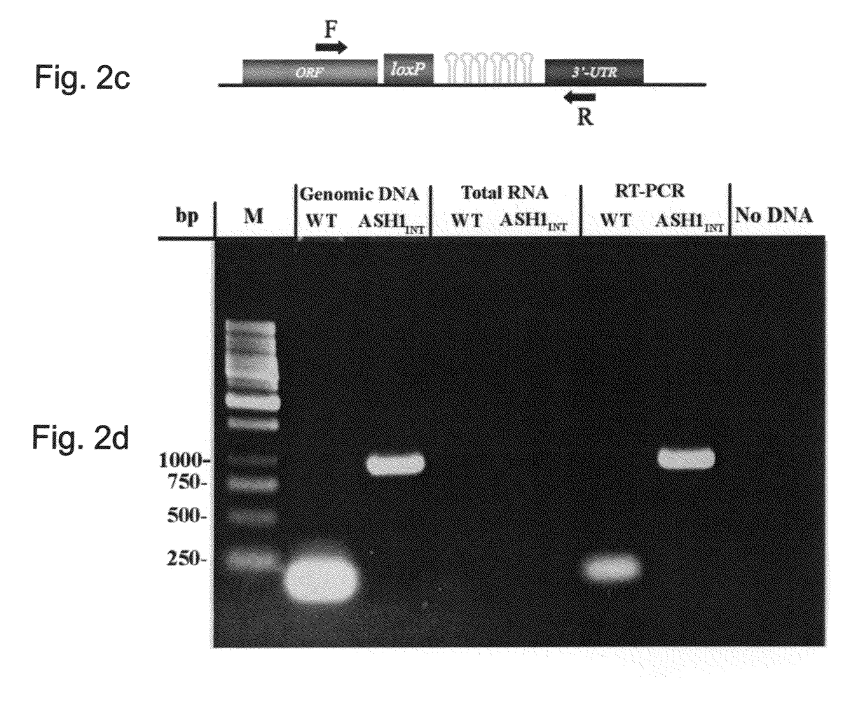

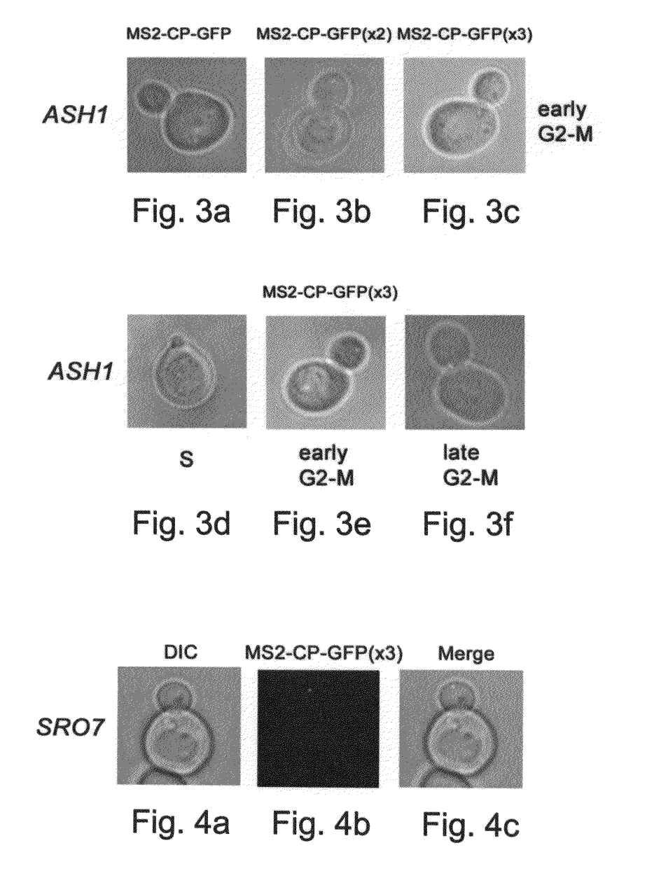

As is shown in FIGS. 1a and 2a-b and is described in Example 1 of the Examples section which follows, the present inventors have constructed a polynucleotide which includes a protein-binding RNA sequence between a portion of the coding sequence and the 3′-UTR of the gene-of-interest such that following homologous recombination in the genome of yeast cells the protein-binding RNA sequence is transcribed under the transcriptional control of the endogenous gene-of-interest. For visualization of the mRNA from a given gene-of-interest, the cells were further transfected with an expression vector encoding the RNA-binding protein fused to GFP (e.g., three copies of the GFP coding sequence). Thus, as is further shown in FIGS. 3a-f, 4a-c, 5a-l, 6a-l, 7a-x and 8a-t and is described in Examples 2, 5, 6 and 7 of the Examples section which follows, the present inventors have demonstrated, for the first time, the localization of the endogenous ASH1, SRO7, PEX3, OXA1, PEX14, PEX13, PEX11, PEX15, PEX1, PEX5, AAT2, GPD1, DC11, POX1, PCS60, MDH3, PCD1, PEX12 and POT1 RNA molecules within living cells.

In addition, the present inventors have uncovered a construct which enables the localization of a polypeptide expressed from a gene-of-interest (see FIG. 1c and Example 4 of the Examples section which follows) as well as a construct which enables the localization of both an mRNA- and a polypeptide expressed from the gene-of-interest in living cells under endogenous transcriptional control (see FIG. 1b and Example 3 of the Examples section which follows).

Since a gene-of-interest may encode several RNA isoforms (e.g., splice variants) and/or several polypeptide isoforms (e.g., variants of different size and structure), the present invention envisages the detection of all RNA and/or polypeptide isoforms encoded by a gene-of-interest which share a common nucleic acid sequence that is used for integration of the polynucleotide within the cell genome. For example, such a common nucleic acid sequence can be on one hand the 3′-end of the coding sequence of the gene-of-interest (e.g., a portion of the last coding exon) and/or the very 5′-end of the 3′-UTR of the gene-of-interest.

Thus, according to one aspect of the present invention there is provided an isolated polynucleotide comprising a first nucleic acid sequence which comprises two functionally compatible recognition sites for a site-specific recombination enzyme and a second nucleic acid sequence encoding a protein binding-RNA sequence.

As used herein the phrase “functionally compatible recognition sites for a site-specific recombination enzyme” refers to specific nucleic acid sequences which are recognized by a site-specific recombination enzyme to allow site-specific DNA recombination (i.e., a crossover event between homologous sequences). An example of a site-specific recombination enzyme is the Cre recombinase (e.g., GenBank Accession No. YP_{—}006472), which is capable of performing DNA recombination between two loxP sites [e.g., a loxP site is set forth by SEQ ID NO:98 (ATAACTTCGTATAATGTATGCTATACGAAGTTAT)]. Cre recombinase can be obtained from various suppliers such as the New England BioLabs, Inc, Beverly, Mass., or it can be expressed from a nucleic acid construct in which the Cre coding sequence is under the transcriptional control of an inducible promoter (e.g., the galactose-inducible promoter) as in plasmid pSH47 used by the present inventors (see the Examples section which follows).

As mentioned, the second nucleic acid sequence encoding a protein binding-RNA sequence. As used herein the phrase “protein binding-RNA sequence” refers to an RNA sequence which serves as a binding site for an RNA binding-protein. Preferably, the RNA sequence forms a secondary structure (e.g., a stem-loop structure) which can bind to a specific domain of the RNA binding-protein. Preferably, the length of the protein binding-RNA sequence is less than 100 nucleic acids, more preferably, less than 50 nucleic acids, even more preferably, between 15 and 25 nucleic acids. Preferably, the binding interaction between the protein binding-RNA sequence and the specific domain of the RNA binding-protein displays high specificity, which results in a high signal-to-noise ratio. In addition, it will be appreciated that the second nucleic acid sequence which encodes the protein binding-RNA sequence can include more than one copy of the protein binding-RNA sequence (identical or different) in order to increase the interaction between the protein binding-RNA sequence and the RNA binding-protein domain. For example, the second nucleic acid sequence can encode at least 2, more preferably, between 6-24 copies of the protein binding-RNA sequence.

A preferred protein binding-RNA sequence is the bacteriophage MS2 binding site (AAACATGAGGATCACCCATGT; SEQ ID NO:94). Complete MS2 nucleotide sequence information can be found in Fiers et al., Nature 260:500-507 (1976). Additional information concerning the MS2 sequence-specific protein-RNA binding interaction appears in Valegard et al., J. Mol. Biol. 270:724-738 (1997); Fouts et al., Nucleic Acids Res. 25:4464-4473 (1997); and Sengupta et al., Proc. Natl. Acad. Sci. USA 93:8496-8501 (1996). The number of copies of the MS2-CP binding stem-and-loop sequence included in the second nucleic acid sequence may vary and can be, for example, 6, 12, and 24 copies. For example, the second nucleic acid sequence used by the present invention (SEQ ID NO:101) includes 12 copies of sequence encoding the MS2 stem-and-loop structure (SEQ ID NO:94; see FIG. 1a and description in the “General Materials and Experimental Methods” of the Examples section which follows).

Other pairs of protein binding-RNA sequence/RNA binding-protein domain which can be used along with this aspect of the present invention include the hairpin II of the U1 small nuclear RNA and the RNA-binding domain of the U1A spliceosomal protein (Oubridge et al., Nature 372:432-438 (1994); the IRP1 protein and the IRE target RNA sequence (a stem-loop structure found in the untranslated regions of mRNAs encoding certain proteins involved in iron utilization) [Klausner et al., Cell 72:19-28 (1993); Melefors et al., Bioessays 15:85-90 (1993)]; the HIV REV and RRE (Zapp & Green, Nature 342:714-716 (1989); Heaphy et al., Cell 60:685-693 (1990); Malim et al. Cell 60:675-683 (1990)]; the zipcode binding protein and the zipcode RNA element (Steward et al., in mRNA Metabolism and Posttranscriptional

Gene Regulation, Wiley-Liss, New York, 127-146); and the box C/D motif and box C/D snoRNA family-specific binding protein [Samarsky et al., EMBO J. 17:3747-3757, (1998)].

In addition, the protein binding-RNA sequence can be an aptamer produced by in vitro selection. An aptamer that binds to a protein (or binding domain) of choice can be produced using conventional techniques, without undue experimentation, essentially as described in Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996).

As used herein the phrase “isolated polynucleotide” refers to a nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA-dependent DNA polymerase. Such a sequence can be subsequently amplified in vivo or in vitro using a DNA-dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode an RNA or a polypeptide encoded by a gene-of-interest, as well as some intronic sequences interposing therebetween. The intronic sequences can be of any source, including of other genes, and typically will include conserved splicing signal sequences. Such intronic sequences may further include cis acting expression regulatory elements.

Preferably, the first and the second nucleic acid sequences of the isolated polynucleotide of this aspect of the present invention are sequentially arranged, i.e., are arranged such that the first nucleic acid sequence is positioned upstream of the second nucleic acid sequence. It will be appreciated that additional nucleic acid sequences such as linkers (which join the segments of the polynucleotide) can be placed between the first and second nucleic acid sequences without affecting the functional activity of the isolated polynucleotide in homologous recombination with genomic sequences. Non-limiting examples of such linkers are provided in nucleic acids 1435-1442 of SEQ ID NO:103.

Preferably, the first nucleic acid sequence further comprises a selectable marker. Such a selectable marker can be any nucleic acid sequence which when transformed or integrated into a cell imparts the cell an advantage (i.e., a positive selection marker) or a disadvantage (i.e., a negative selection marker) according to which the cell can be selected. Non-limiting examples of selectable markers include drug-resistance genes (e.g., antibiotic resistance genes such as Kanamycin resistance, Ampicillin resistance, G418 resistance, and Hygromycin resistance), genes encoding polypeptides (e.g., His3, Ura3, Trp1 , Leu2, and Ade2) which participate in the biosynthesis of an essential nutrient and enable a cell having such a marker to grow on a medium devoid of such a nutrient, or a lethal marker (e.g., a thymidine kinase) which when present in a cell causes cell death, and genes encoding visual markers (e.g. green fluorescent protein (GFP) for fluorescence imaging or an eye color marker—white; w⁺ for use in the selection of Drosophila). For example, a suitable marker for selecting cells (e.g., yeast cells) which underwent homologous recombination with the isolated polynucleotide of the present invention is a marker that participates in the biosynthesis of an essential nutrient. Thus, cells are cultured in the presence of a culture medium devoid of the essential nutrient and only cells in which the isolated polynucleotide has integrated in the genome are capable of growing. Additionally or alternatively, a suitable marker for selecting prokaryotic (e.g., bacteria) or other eukaryotic cells (e.g., Drosophila or mammalian cells, such as mouse or human) which underwent homologous recombination with the isolated polynucleotide of the present invention can be a marker conferring drug-resistance, such as ampicillin-, Kanamycin-, G418-, and hygromycin-resistance; genetic selection (e.g. eye color selection in Drosophila); or selection based upon fluorescence. Thus, in the case of selection for antibiotic-resistance cells (e.g., mouse or human embryonic stem cells) are cultured in the presence of a culture medium including the drug (e.g., antibiotic) and only cells in which the isolated polynucleotide has integrated in the genome are capable of growing. Likewise, cells bearing the GFP marker can be identified and sorted using fluorescence-activated cell sorting, while Drosophila bearing the white gene can be identified by visual inspection.

Preferably, the selectable marker included in the first nucleic acid sequence of the isolated polynucleotide of the present invention is positioned (placed) between the two recognition sites for the site-specific recombination enzyme such that following induction of site-specific recombination the marker can be excised from the isolated polynucleotide. For example, when homologous recombination is performed with a Cre recombinase, a selectable marker which is positioned between the two parallel loxP sites of the first nucleic acid sequence is removed, leaving the isolated polynucleotide with only one loxP site.

It will be appreciated that the removal of the selectable marker is advantageous in order to enable the endogenous 3′-UTR sequence to control the correct RNA trafficking (e.g., mRNA trafficking) and prevent mis-targeting of the mRNA encoded by the gene-of-interest within the cells. Thus, a presence of a long sequence (e.g., 2 kb) of a selectable marker can hamper the natural transcriptional regulation of the RNA encoded by the gene-of-interest.

Preferably, to enable homologous recombination of the isolated polynucleotide of the present invention into a genomic sequence of the gene-of-interest, the isolated polynucleotide further comprising additional nucleic acid sequences (e.g., a third and a forth nucleic acid sequences) which correspond to endogenous sequences of the gene-of-interest.

For example, a third nucleic acid sequence can correspond to a portion of the coding sequence of the RNA molecule encoded by the gene-of-interest (e.g., a portion at the 3′-end of the coding sequence) such that a cross over event will occur at this sequence.

In addition, a fourth nucleic acid sequence can correspond to a portion of the 3′- UTR of the genomic sequence of the gene-of-interest, preferably, to a sequence derived from the 5′-end of the 3′-UTR sequence [e.g., the nucleic acid sequence which immediately follows the stop codon of the encoded polypeptide by the gene-of-interest]. It will be appreciated that to enable homologous recombination between the isolated polynucleotide of the present invention and the genomic sequence encoding the mRNA/polypeptide of the gene-of-interest, the third nucleic acid sequence is preferably positioned upstream of the first nucleic acid sequence of the isolated polynucleotide and the fourth nucleic acid sequence is preferably positioned downstream of the second nucleic acid sequence of the isolated polynucleotide. It will be appreciated that since homologous recombination in mammalian cells requires longer flanking sequences as compared to those needed in yeast cells, to enable homologous recombination in mammalian cells the third and forth nucleic acid sequences of the invention may include several hundreds or thousands of nucleic acids.

As mentioned hereinabove and is described in FIG. 1b and Example 3 of the Examples section which follows, the present inventors have uncovered that the localization of a polypeptide encoded by a gene-of-interest can be visualized along with the mRNA of the same gene-of-interest in living cells by inserting an isolated polynucleotide designed to tag both the endogenously transcribed mRNA and the endogenously translated protein.

Thus, according to an additional aspect of the present invention there is provided an isolated polynucleotide comprising a first nucleic acid sequence which comprises two functionally compatible recognition sites for a site-specific recombination enzyme, a second nucleic acid sequence encoding a protein binding-RNA sequence and a third nucleic acid sequence encoding a reporter polypeptide.

As used herein the phrase “reporter polypeptide” refers to any polypeptide which can be detected in a cell. Preferably, the reporter polypeptide of this aspect of the present invention can be directly detected in the cell (no need for a detectable moiety with an affinity to the reporter) by exerting a detectable signal which can be viewed in living cells (e.g., using a fluorescent microscope). Non-limiting examples of a nucleic acid sequence encoding a reporter polypeptide according to this aspect of the present invention include the red fluorescent protein (RFP) (e.g., SEQ ID NO:100) or the green fluorescent protein (GFP) (e.g., SEQ ID NO:99).

Alternatively, the reporter polypeptide can be indirectly detected such as when the reporter polypeptide is an epitope tag. Indirect detection can be effected by introducing a detectable moiety (labeled antibody) having an affinity to the reporter or when the reporter is an enzyme by introducing a labeled substrate. For example, the reporter polypeptide can be an antigen which is recognized by and binds to a specific antibody. Preferably, when such a reporter polypeptide is utilized the antibody or the polypeptide capable of binding the reporter protein is labeled (e.g., by covalently attaching to a label such as a fluorescent dye).

Preferably, the first and the second nucleic acid sequences of the isolated polynucleotide of this aspect of the present invention are sequentially arranged. More preferably, the third nucleic acid sequence is positioned upstream of the first nucleic acid sequence.

Preferably, to enable homologous recombination, the isolated polynucleotide of this aspect of the present invention further includes additional nucleic acid sequences corresponding to a portion of the coding sequence and the 3′-UTR of the gene-of-interest, essentially as described in the Examples section which follows.

Thus, the present invention provides a transformed cell having a genome which comprises an exogenous polynucleotide being transcriptionally regulated by endogenous 5′ and 3′-UTRs of the gene-of-interest, the exogenous polynucleotide comprising a first nucleic acid sequence which comprises at least one recognition site for a site-specific recombination enzyme, and a second nucleic acid sequence encoding a protein binding-RNA sequence and/or a third nucleic acid sequence encoding a reporter polypeptide.

Preferably, within the transformed cell, the expression of the exogenous polynucleotide is regulated by the endogenous 5′ and 3′-UTRs of the gene-of-interest.

In addition, the present invention further envisages the use of an isolated polynucleotide for tagging a polypeptide encoded by a gene-of-interest in living cells, as described in FIG. 1c and Example 4 of the Examples section which follows. Thus, the isolated polynucleotide is inserted via homologous recombination between the endogenous coding sequence and 3′-UTR of the gene-of-interest, such that transcription and localization of the mRNA which is translated to generate the polypeptide (encoded by the gene-of-interest) is under the control of the endogenous sequences, leading to normal mRNA trafficking and subsequently normal polypeptide targeting within the cell.

Thus, according to an additional aspect of the present invention, there is provided a transformed cell having a genome which comprises an exogenous polynucleotide being transcriptionally regulated by endogenous 5′ and 3′-UTRs of the gene-of-interest, the exogenous polynucleotide comprising a first nucleic acid sequence which comprises at least one recognition site for a site-specific recombination enzyme, and a second nucleic acid sequence encoding a reporter polypeptide.

It will be appreciated that localization of the RNA and/or the polypeptide encoded by a gene-of-interest may be further achieved by a polynucleotide system which includes two polynucleotides capable of homologous recombination: one which can localize the RNA (e.g., mRNA) encoded by the gene-of-interest and the second, which can localize the polypeptide encoded by the gene-of-interest.

Thus, according to yet an additional aspect of the present invention there is provided a system of isolated polynucleotides. The system comprising: (i) a first isolated polynucleotide comprising a first nucleic acid sequence which comprises two functionally compatible recognition sites for a site-specific recombination enzyme and a second nucleic acid sequence encoding a protein binding-RNA sequence; and (ii) a second isolated polynucleotide comprising a first nucleic acid sequence which comprises two functionally compatible recognition sites for a site-specific recombination enzyme and a second nucleic acid sequence encoding a reporter polypeptide.

To obtain large amounts of any of the isolated polynucleotides described hereinabove or the system containing same, the isolated polynucleotide is preferably ligated into a nucleic acid construct.

The nucleic acid construct (also referred to herein as an “expression vector”) of the present invention may include additional sequences that render this vector suitable for replication and integration in prokaryotes, eukaryotes, or preferably both (e.g., shuttle vectors). In addition, a typical cloning vector may also contain transcription and translation initiation sequences, transcription and translation terminators, and a polyadenylation signal.

In addition to the embodiments already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote extra-chromosomal replication of the viral genome in permissive cell types. Plasmids bearing these viral replicons are replicated episomally as long as the appropriate factors are provided by genes either carried on the plasmid or with the genome of the host cell.

The expression vector of the present invention may or may not include a eukaryotic replicon. If a eukaryotic replicon is present, the vector is capable of amplification in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(±), pGL3, pZeoSV2(±), pSecTag2, pDisplay, pEF/myc/cyto, pCMV/myc/cyto, pCR3.1, pSinRep5, DH26S, DHBB, pNMT1, pNMT41, and pNMT81, which are available from Invitrogen, pCI which is available from Promega, pMbac, pPbac, pBK-RSV and pBK-CMV, which are available from Strategene, pTRES which is available from Clontech, and their derivatives.

Examples of yeast expression vectors containing constitutive or inducible promoters are disclosed in U.S. Pat. No: 5,932,447; Sikorski & Hieter, Genetics 122:19-27 (1989) and Christianson et al. Gene 110;119-122 (1992).

Expression vectors containing regulatory elements from eukaryotic viruses such as retroviruses can be also used. SV40 vectors include pSVT7 and pMT2, for instance. Vectors derived from bovine papilloma virus include pBV-1MTHA, and vectors derived from Epstein-Barr virus include pHEBO and p2O5. Other exemplary vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

It will be appreciated that any of the isolated polynucleotide, nucleic acid constructs and/or systems thereof described hereinabove can be used to transform cells by methods well known in the art.

The present invention further provides a method of identifying the localization of an RNA and/or a polypeptide encoded by a gene-of-interest within a cell.

The method is effected by: (a) introducing into the cell the isolated polynucleotide of the present invention, so as to enable homologous recombination of the isolated polynucleotide between endogenous 5′ and 3′-UTRs of the gene-of-interest; (b) detecting the RNA encoded by the gene-of-interest via the protein binding-RNA sequence; and/or (c) detecting the reporter polypeptide.

Methods of introducing isolated polynucleotides into cells are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, lipofection and electroporation.

Detecting RNA (e.g., mRNA) localization via the protein binding-RNA sequence can be performed by either expressing within or introducing to the cell (which underwent homologous recombination with the isolated polynucleotide of the present invention) a polypeptide capable of binding the protein binding-RNA sequence. Non-limiting examples of such polypeptides are described hereinabove and in the Examples section which follows. For example, to detect the MS2L protein-binding RNA sequence the present inventors have expressed the coding sequence of the MS2 coat protein (SEQ ID NO:102) in yeast cells which were subjected to homologous recombination with the isolated polynucleotide of the present invention [e.g., the polynucleotide including a portion of the ASH1 3′-UTR with the MS2L RNA sequence (SEQ ID NO:101)]. Additionally or alternatively, the RNA binding protein itself can be administered to the cells (e.g., the MS2 coat protein set forth by GenBank Accession No. NP_{—}040648) and bind to the MS2L protein-binding RNA sequence.

Preferably, the polypeptide capable of binding the protein-binding RNA sequence is labeled (i.e., attached to a label). Such a labeled polypeptide can be obtained by forming a fusion protein containing the coding sequence of a polypeptide capable of binding the protein-binding RNA sequence and of a polypeptide capable of exerting a fluorescent signal such as the green fluorescent protein (GFP). It will be appreciated that the coding sequence of the polypeptide capable of binding the protein-binding RNA sequence can be expressed from a constitutive or inducible exogenous promoter, or from the promoter sequence derived from the genomic sequence of the gene-of-interest (which encodes the RNA and/or the polypeptide to be localized within the cell) in order to correlate co-transcription of both the RNA encoded by the gene-of-interest and the coding sequence of the RNA binding protein. A non-limiting example of such a labeled polypeptide is the polypeptide expressed from the pMS2-CP-GFP(x3) nucleic acid construct (SEQ ID NO:92) which encodes the MS2 coat protein (SEQ ID NO:102) along with three copies of the GFP coding sequence (SEQ ID NO:99), essentially as described in the Examples section which follows. It will be appreciated that such a labeled polypeptide can be viewed using a fluorescent microscope. Since the polypeptide capable of binding the protein-binding RNA sequence is labeled even without binding to the protein binding-RNA sequence, measures are taken in order to discriminate between the background labeling obtained in the whole cell and the punctuated labeling obtained within the specific localization of the RNA encoded by the gene-of-interest (See FIGS. 3a-f, 4a-c, 5a-l and 6a-l). It should be noted that various known algorithms can be used in order to automatically subtract the background labeling from the labeling corresponding to the expression of the RNA encoded by the gene-of-interest and those of skills in the art know how to implement such algorithms to any image analysis system.

As mentioned, the reporter polypeptide can exert a detectable moiety such as red fluorescence. Thus, detection of the reporter polypeptide (described hereinabove) can be performed using methods known in the art such as by a fluorescent microscope (e.g., a confocal microscope).

Preferably, the cell used by the method of this aspect of the present invention is capable of homologous recombination or is modified to allow homologous recombination. Such a cell is preferably a eukaryotic cell such as a mammalian cell, yeast cell, and a plant cell.

Preferably, identification of the localization of the RNA and/or the polypeptide encoded by the gene-of-interest is performed in a living cell, i.e., while the cell is still alive and is capable of proliferation, differentiation and metabolism of nutrients

It will be appreciated that the method of identifying the localization of the RNA and/or the polypeptide encoded by the gene-of-interest may be used in a high throughput process for the localization of all mRNAs and/or polypeptides within the cell. Thus, specific pairs of primers can be prepared in order to PCR amplify the isolated polynucleotide of the present invention along with the additional gene-specific sequences (e.g., which are derived from the 3′-end of the coding sequence and the 5′-end of the 3′-UTR of the gene-of-interest). The amplified PCR products can be introduced into cells and undergo homologous recombination with the cell genome. It will be appreciated that for the detection of mRNA encoded by each gene-of-interest a unique pair of protein binding RNA sequence and an RNA binding protein attached to a specific label can be used. The specific labels used can be, for example, RFP, GFP, yellow fluorescent protein (YFP), cyano fluorescent protein (CFP) and variants thereof which exhibit non-overlapping emission spectra and thus can be distinguished when applied in a single cell.

The present invention further provides kits for localization of an mRNA and/or a polypeptide encoded by a gene-of-interest. Such a kit includes (i) the isolated polynucleotide of the present invention, and (ii) a pair of oligonucleotides which enable homologous recombination of the isolated polynucleotide between endogenous 5′ and 3′-UTRs of the gene-of-interest.

Thus, for localization of an mRNA encoded by a given gene-of-interest, the kit includes a specific pair of oligonucleotides which enable homologous recombination of the isolated polynucleotide (which includes a first nucleic acid sequence which comprises two functionally compatible recognition sites for a site-specific recombination enzyme and a second nucleic acid sequence encoding a protein binding-RNA sequence) between endogenous 5′ and 3′-UTRs of the gene-of-interest. For example, for localization of ASH1 RNA, such a kit includes the pair of oligonucleotides set forth by SEQ ID NOs:1 and 2; for localization of SRO7 RNA, such a kit includes the pair of oligonucleotides set forth by SEQ ID NOs:3 and 4; for localization of OXA1 RNA, such a kit includes the pair of oligonucleotides set forth by SEQ ID NOs:5 and 6; for localization of PEX3 RNA, such a kit includes the pair of oligonucleotides set forth by SEQ ID NOs:7 and 8; for localization of SNC1 RNA, such a kit includes the pair of oligonucleotide set forth by SEQ ID NOs:9 and 10; for localization of DCI1 RNA, such a kit includes the pair of oligonucleotide set forth by SEQ ID NOs:11 and 12; for localization of FOX2 RNA, such a kit includes the pair of oligonucleotides set forth by SEQ ID NOs: 13 and 14; for localization of PCS60 RNA, such a kit includes the pair of oligonucleotides set forth by SEQ ID NOs:15 and 16; for localization of PEX1 RNA, such a kit includes the pair of oligonucleotides set forth by SEQ ID NOs: 17 and 18; for localization of PEX14 RNA, such a kit includes the pair of oligonucleotides set forth by SEQ ID NOs:19 and 20; for localization of PEX13 RNA, such a kit includes the pair of oligonucleotides set forth by SEQ ID NOs:106 and 107; for localization of PEX11 RNA, such a kit includes the pair of oligonucleotides set forth by SEQ ID NOs:108 and 109; for localization of PEX15 RNA, such a kit includes the pair of oligonucleotides set forth by SEQ ID NOs:21 and 22; for localization of PEX5 RNA, such a kit includes the pair of oligonucleotides set forth by SEQ ID NOs:31 and 32; for localization of AAT2 RNA, such a kit includes the pair of oligonucleotides set forth by SEQ ID NOs:110 and 111; for localization of GPD1 RNA, such a kit includes the pair of oligonucleotides set forth by SEQ ID NOs:112 and 113; for localization of POX1 RNA, such a kit includes the pair of oligonucleotides set forth by SEQ ID NOs:35 and 36; for localization of MDH3 RNA, such a kit includes the pair of oligonucleotides set forth by SEQ ID NOs:132 and 133; for localization of PCD1 RNA, such a kit includes the pair of oligonucleotides set forth by SEQ ID NOs:136 and 137; for localization of PEX12 RNA, such a kit includes the pair of oligonucleotides set forth by SEQ ID NOs:150 and 151; for localization of POT1 RNA, such a kit includes the pair of oligonucleotides set forth by SEQ ID NOs:33 and 34. Additional pairs of oligonucleotides which can be used for the localization of other RNAs encoded by genes-of-interest are provided in Table 1 of the Examples section which follows.

Additionally or alternatively, when the kit is used for identifying the localization of a polypeptide encoded by a gene-of-interest (without the localization of the mRNA encoded by the same gene-of-interest), such a kit includes a specific pair of oligonticleotides which enable homologous recombination of the isolated polynucleotide (which includes a first nucleic acid sequence which comprises at least one recognition site for a site-specific recombination enzyme, and a second nucleic acid sequence encoding a reporter polypeptide) between endogenous 5′ and 3′-UTRs of a genomic sequence encoding the polypeptide of the gene-of-interest. For example, for localization of Ash1 protein such a kit includes the pair of oligonucleotides set forth by SEQ ID NOs:91 and 2; for localization of Sro7 protein such a kit includes the pair of oligonucleotides set forth by SEQ ID NOs:93 and 4; for localization of Oxa1 protein such a kit includes the pair of oligonucleotides set forth by SEQ ID NOs:95 and 6; and for localization of Pex3 protein such a kit includes the pair of oligonucleotides set forth by SEQ ID NOs:97 and 8 (see Table 4 of the Examples section which follows).

Still additionally or alternatively, when the kit is used for identifying the localization of both the mRNA and the polypeptide encoded by the gene-of-interest, such a kit includes a specific pair of oligonucleotides which enable homologous recombination of the isolated polynucleotide (which includes a first nucleic acid sequence which comprises two functionally compatible recognition sites for a site-specific recombination enzyme, a second nucleic acid sequence encoding a protein binding-RNA sequence and a third nucleic acid sequence encoding a reporter polypeptide) between endogenous 5′ and 3′-UTRs of the gene-of-interest. For example, for co-localization of Ash1 RNA and protein such a kit includes the pair of oligonucleotides set forth by SEQ ID NOs:91 and 2; for co-localization of Sro7 RNA and protein such a kit includes the pair of oligonucleotides set forth by SEQ ID NOs:93 and 4; for co-localization of Oxa1 RNA and protein such a kit includes the pair of oligonucleotides set forth by SEQ ID NOs:95 and 6; and for co-localization of Pex3 RNA and protein such a kit includes the pair of oligonucleotides set forth by SEQ ID NOs:97 and 8 (see Table 4 of the Examples section which follows).

Preferably, the kit further comprising a reagent for detecting the protein binding-RNA sequence [e.g., GFP(x3) conjugated to the RNA-binding protein described hereinabove and in the Examples section which follows] and/or the reporter polypeptide (e.g., the mRFP protein).

In addition, the kit may further include reagents suitable for PCR amplification of the isolated polynucleotide with the pair of oligonucleotides. Such reagents can be Taq polymerase and suitable buffers.

The compositions included in the kit of the present invention (e.g., the isolated polynucleotides, pairs of oligonucleotides) may be presented in a pack or dispenser device. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and

Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N. Y. (1994), Third Edition; “Current Protocols in

Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Materials and Experimental Methods

Media, DNA and Genetic Manipulations—Yeast were grown in standard growth media containing either 2% glucose or 3.5% galactose. Synthetic complete (SC) and drop-out media were prepared similar to that described elsewhere (24). Standard methods were used for the introduction of DNA into yeast and the preparation of genomic DNA (24).

Plasmids—Plasmid pUG27 (Euroscarf; Universitat Frankfurt, Frankfurt, Germany), which contains the loxP-SpHIS5-loxP cassette, was used as the vector backbone to create the template plasmid for generating integration constructs by PCR. A multicopy plasmid expressing Sec63-RFP, pSM1960, was generously provided by S. Michaelis (John Hopkins, Baltimore, Md., USA). Plasmid pSL-MS2-12X, which contains 12 tandem MS2 loop sequences, was provided by R. Singer (Albert Einstein College of Medicine, NY). Plasmid pSL-MS2-12X was altered by Pfu mutagenesis to add an EcoRV site 5′ to the MS2 loop sequence and yielded plasmid p12MS2L-RV. Next, a 694 bp fragment containing 12 MS2 loops was excised from p12MS2L-RV using EcoRV (which cuts at EcoRV sites 5′ and 3′ to the loops) and inserted in the correct orientation into the EcoRV site located downstream of the second loxP sequence in pUG27 to yield the template plasmid for mRNA localization—pLOXHIS5MS2L. The template plasmid for protein and mRNA localization, pRFPLOXHIS5MS2L, was created by first amplifying mRFP (lacking its start codon) from pRSET-B/RFP (provided by R. Tsien, UCSD, CA) using a forward oligonucleotide containing a HindIII site and a reverse oligonucleotide complementary to a sequence in the plasmid downstream of mRFP. The mRFP gene in pRSET-B/RFP contains a HindIII site downstream of its stop codon. The PCR-amplified fragment was cloned into pGEM-Teasy (Promega) to yield plasmid pRFP-HIII. Next, a 700 bp HindIII fragment was excised from pRFP-HIII and cloned (in the correct orientation) into the HindIII site situated upstream to the first loxP site in pLOXHIS5MS2L to yield pRFPLOXHIS5MS2L. A 694 bp fragment containing MS2L was excised from pRFPLOXHIS5MS2L using EcoRV and the vector re-ligated to yield pRFPLOXHIS5. Plasmid pSH47, which expresses CRE recombinase from a galactose-inducible promoter, was obtained from Euroscarf. Plasmid pCP-GFP1, which expresses MS2-CP fused with GFP under the MET25 promoter was provided by K. Bloom (U. North Carolina, Chapel Hill, N.C.). A double GFP MS2-CP fusion (MS2-CP-GFP(x2)) was created by first amplifying GFP from pCP-GFP using oligonucleotides containing EcoRV sites and cloning into pGEM-Teasy (Promega) to yield plasmid pGFP-RV. Next, a 721 bp EcoRV fragment was cloned (in the correct orientation) into the EcoRV site situated between MS2-CP and GFP in pCP-GFP to yield pMS2CPGFP(x2). A triple GFP MS2-CP fusion (MS2-CP-GFP(x3)) was created by eliminating the 3′ EcoRV site (situated between the two GFP genes) in pMS2CPGFP(x2), by site-directed mutagenesis with Pfu polymerase, and subsequent insertion of GFP into the 5′ EcoRV site. A plasmid expressing OXA1-mRFP was created by first amplifying OXA1 bp PCR and subsequent subcloning into the SalI-SmaI site of pAD4Δ, a 2u vector bearing the LEU2 selection marker and ADH1 promoter, to yield pAD4Δ-OXA 1 . Next, a PCR-amplified fragment encoding monomeric RFP (mRFP) was subcloned into the SmaI-SacI sites of pAD4Δ-OXA1 to yield pAD4Δ-OXA1-RFP; following which 500 bp of the OXA1 3′-UTR was subcloned in its correct orientation into the SacI site of pAD4Δ-OXA 1-RFP to yield pAD4Δ-OXA1-RFP-3′-UTR. All constructs were verified by sequencing.

Genomic integration of either MS2-CP binding sites or mRFP and MS2-CP binding sites into yeast—The integration constructs described above (pLOXHIS5MS2L, pRFPLOXHIS5MS2L, and pRFPLOXHIS5) can be used for the tagging of any yeast gene of interest by PCR amplification using specific oligonucleotide primers (for a given gene) to generate the DNA integration fragment. For mRNA tagging alone, the forward primer for MS2L tagging includes a sequence complementary to the 3′ end of the coding region (overlapping by ˜40 bp and including the stop codon) and the 5′ end of the loxP::SpHIS5::loxP::MS2L cassette in pLOXHIS5MS2L. For dual mRNA and protein tagging or protein tagging alone, the forward primer includes sequence complementary to the 3′ end of the coding region of the gene of interest (overlapping by ˜40 bp and lacking the native stop codon) and the 5′ end of the mRFP sequence. In all cases, a reverse oligonucleotide complementary (by ˜40 bp) to the 5′ end of the 3′-UTR and 3′ end of the cassette was used in the PCR reaction with pLOXHIS5MS2L, pRFPLOXHIS5MS2L, or pRFPLOXHIS5 as templates for mRNA tagging, mRNA and protein tagging, and protein tagging, respectively (see FIGS. 1a-c for a schematic representations, respectively). PCR products of the correct size were transformed into wild-type yeast and grown on plates containing SC medium lacking histidine for 3-5 days in 26° C. To confirm integration, genomic DNA was extracted from single colonies and PCR amplification, using a forward primer complementary to the coding region and reverse primer complementary to the loxP::SpHIS5::loxP::MS2L cassette (in the case of mRNA tagging); mRFP::loxP::SpHIS5::loxP::MS2L cassette (in the case of mRNA and protein tagging); or the mRFP::loxP::SpHIS5::loxP cassette (in the case of protein tagging alone) and the 3′-UTR, was performed. PCR products were sized on agarose gels and sequenced for verification. Yeast bearing correct loxP::SpHIS5::loxP::MS2L integrations were transformed with pSH47 and grown on SC medium lacking histidine and uracil.

Cre recombinase expression was induced by growing transformed cells in SC medium containing galactose and lacking uracil for 16 hours in 26° C. Cells were then diluted, plated and grown on SC medium lacking uracil, and replica plated to determine the presence or absence of the SpHIS5 auxotrophic marker. Yeast bearing the loxP::MS2L integration, mRFP::loxP::MS2L, and mRFP::loxP were verified by PCR amplification (using oligonucleotides complementary to the coding region and 3′-UTR, respectively) and DNA sequencing.

Finally, total RNA was purified from both wild-type and the loxP::MS2L integrated yeast strains using the Masterpure™ Yeast RNA purification kit (include DNase treatment). Total RNA was resuspended in 30 μl A DEPC-treated water and 1 μg aliquots were taken for the reverse transcription using M-MLV RT RNase H (−) (Promega). To detect specific transcripts, 40 ng of transcribed RNA was amplified by PCR using specific oligonucleotides.

MS2-CP-GFP expression and mRNA/protein visualization—Integrated loxP::MS2L and mRFP::loxP::MS2L strains were transformed with plasmids expressing MS2-CP-GFP, MS2-CP-GFP(x2) or MS2-CP-GFP(x3) and fusion protein expression induced by growth for 1 hour at 26° C. in synthetic medium lacking methionine. Cells were examined by fluorescence microscopy to visualize mRNA (by GFP fluorescence) or protein (by RFP fluorescence).

TABLE 1
Primers used for genomic integration of
MS2L-mRNA localization cassette

| | GenBank | |
|---|---|---|
| | Accession No. | Primer sequence |
| mRNA | (position) | (SEQ ID NO: ) (5′→3′) |
| ASH1 | NC_001143 | F (SEQ ID NO: 1): |
| | (94504-96270) | CTTATTTTGTAATTACATAACTGA |
| | | GACAGTAGAGAATTGAACGCTGCAGG |
| | | TCGACAACCC |
| | | R (SEQ ID NO: 2): |
| | | ATGTCTCTTATTAGTTGAAAGAGA |
| | | TTCAGTTATCCATGTAGCATAGGCCA |
| | | CTAGTGGATC |
| SR07 | NC_001148 | F (SEQ ID NO: 3): |
| | (634120-637221) | GAGCAGACTGGAAAAGATGTAATG |
| | | AAAGGTGCCCTTGGTTTTTAAACGCT |
| | | GCAGGTCGACAACCC |
| | | R (SEQ ID NO: 4): |
| | | ATAGAAGGAAGTTGCTCATTACCC |
| | | TGTATGAATTAGTGTATGTATCTGAT |
| | | ATCGATCGCGCGCAG |
| OXA1 | NC_001137 | R (SEQ ID NO: 5): |
| | (475015-476223) | AATTGTTCACAAATCAAACTTCAT |
| | | TAATAACAAAAAATGAACGCTGCAGG |
| | | TCGACAACCC |
| | | R (SEQ ID NO: 6): |
| | | TTTATATTTTTATATTTACAGAGA |
| | | GATATAGAGCCTTTATGCATAGGCCA |
| | | CTAGTGGATC |
| PEX3 | NC_001136 | F (SEQ ID NO: 7): |
| | (1127590-1126265) | CAACTTTGGCGTCTCCAGCTCGTT |
| | | TTCCTTCAAGCCTTAAACGCTGCA |
| | | GGTCGACAACCC |
| | | R (SEQ ID NO: 8): |
| | | TATATATATTCTGGTGTGAGTGTC |
| | | AGTACTTATTCAGAGAGCATAGGC |
| | | CACTAGTGGATC |
| SNC1 | NC_001133 | F (SEQ ID NO: 9): |
| | (87287-87753) | TGTAATCATCGTCCCCATTGCTGT |
| | | TCACTTTAGTCGATAGACGCTGCA |
| | | GGTCGACAACCC |
| | | R (SEQ ID NO: 10): |
| | | TATGGAAGCTCCCTATATATATAG |
| | | CATTGCGAGTGAACTTGCATAGGC |
| | | CACTAGTGGATC |
| DCI1 | NC_001147 | F (SEQ ID NO: 11): |
| | (675168-674353) | TAAACAGCTTCAAGAGGGAAACAG |
| | | GCGCCACAAGTTATAAACGCTGCA |
| | | GGTCGACAACCC |
| | | R (SEQ ID NO: 12): |
| | | GATTTTTTATGTTAAAATCCTATC |
| | | TCTAAATGCTATATTAGCATAGGC |
| | | CACTAGTGGATC |
| FOX2 | NC_001143 | F (SEQ ID NO: 13): |
| | (456697-453995) | CGCCGCTGTAAAACTATCGCAGGC |
| | | AAAATCTAAACTATAAACGCTGCA |
| | | GGTCGACAACCC |
| | | R (SEQ ID NO: 14): |
| | | ATAATCGCTATTTTTTATATTATT |
| | | CAAATCTTTTTTTAGCATAGGCCA |
| | | CTAGTGGATC |
| PCS60 | NC_001134 | F (SEQ ID NO: 15): |
| | (668346-666715) | AACTTTTGCTAAGAGCAGCAGAAA |
| | | TAAGAGTAAGTTGTAGACGCTGCA |
| | | GGTCGACAACCC |
| PCS60 | | R (SEQ ID NO: 16): |
| | | TAGAAGCTTTCAGAGAGCATAAAA |
| | | TTGTACAGGATACTGCGCATAGGC |
| | | CACTAGTGGATC |
| PEX1 | NC_001143 | F (SEQ ID NO: 17): |
| | (73870-70739) | GAATTCCATCGACATTGGTAGCCG |
| | | ACTCTCCCTTATGTGAACGCTGCA |
| | | GGTCGACAACCC |
| | | R (SEQ ID NO: 18): |
| | | TTTAAAGGGAAACGCGCTTTGTTC |
| | | TTTTCTTCTTCCTTTGCATAGGCC |
| | | ACTAGTGGATC |
| PEX14 | NC_001139 | F (SEQ ID NO: 19): |
| | (216278-217303) | TGACTGGCAAAATGGACAGGTCGA |
| | | AGACTCCATCCCATAGACGCTGCA |
| | | GGTCGACAACCC |
| | | R (SEQ ID NO: 20): |
| | | CAATTTCCGTTAAAAAACTAATTA |
| | | CTTACATAGAATTGCGGCATAGGC |
| | | CACTAGTGGATC |
| PEX15 | NC_001147 | F (SEQ ID NO: 21): |
| | (247149-248300) | CCAGATTGTAGGGTTGCTAAAACT |
| | | TCTAGCGAGTATATGAACGCTGCA |
| | | GGTCGACAACCC |
| | | R (SEQ ID NO: 22): |
| | | AAATAAGTAGGTAGGGTTTTATAA |
| | | ACTATTCAAATATTTCGCATAGGC |
| | | CACTAGTGGATC |
| PEX18 | NC_001140 | F (SEQ ID NO: 23): |
| | (420075-419224) | TGGTCTTGAGTTCCATGATGTTGA |
| | | AGACAGAATTGCTTAAACGCTGCA |
| | | GGTCGACAACCC |
| | | R (SEQ ID NO: 24): |
| | | CTGAAATTCATGGTTTAAATTAAA |
| | | GAAATTTCAAGGCCCGGCATAGGC |
| | | CACTAGTGGATC |
| PEX19 | NC_001136 | F (SEQ ID NO: 25): |
| | (337277-336249) | CCTTGATAAGGAATTAACCGACGG |
| | | TTGCAAACAACAATAAACGCTGCA |
| | | GGTCGACAACCC |
| | | R (SEQ ID NO: 26): |
| | | GATAATGAACTACTTTTTTTTTTT |
| | | TTTTTTTACTGTTATCATAAATAT |
| | | ATATACCGCATAGGCCACTAGTGG |
| | | ATC |
| PEX21 | NC_001139 | F (SEQ ID NO: 27): |
| | (970058-969192) | TTTCGTCAAGGACGAAATTCACAA |
| | | AGACATACTTGATTGAACGCTGCA |
| | | GGTCGACAACCC |
| | | R (SEQ ID NO: 28): |
| | | GTAGTAGTTACAAGAGGTACAATT |
| | | GTAGAAACTGCCTATAGCATAGGC |
| | | CACTAGTGGATC |
| PEX28 | NC_001140 | F (SEQ ID NO: 29): |
| | (397254-398993) | GATACATCGTGTTATTAAGAATGC |
| | | AACACCAGTAGCATAAACGCTGCA |
| | | GGTCGACAACCC |
| | | R (SEQ ID NO: 30): |
| | | AAGAGGGGTGGGGTTGTAGGTGAA |
| | | GGAAAACAATTGTGCAGCATAGGC |
| | | CACTAGTGGATC |
| PEX5 | NC_001136 | F (SEQ ID NO: 31): |
| | (950559-952397) | CATGGACCTGAAAAGATTTAAAGG |
| | | AGAATTTTCGTTTTGAACGCTGCA |
| | | GGTCGACAACCC |
| PEX5 | | R (SEQ ID NO: 32): |
| | | TGGGCAGTGATGCGAGAACATAAA |
| | | ATTGCGGAGAACATAGCATAGGCC |
| | | ACTAGTGGATC |
| POT1 | NC_001141 | F (SEQ ID NO: 33): |
| | (41444-40191) | TACTGGTATGGGTGCCGCCGCCAT |
| | | CTTTATTAAAGAATAGACGCTGCA |
| | | GGTCGACAACCC |
| | | R (SEQ ID NO: 34): |
| | | AATAAAAAGGGAGAATATTAACTA |
| | | TTATCAAGTATTAAAAGCATAGGC |
| | | CACTAGTGGATC |
| POX1 | NC_001139 | F (SEQ ID NO: 35): |
| | (108162-110408) | TGCAGCTAATGCGGAAATTTTATC |
| | | GAAAATAAACAAGTGAACGCTGCA |
| | | GGTCGACAACCC |
| POX1 | NC_001139 | R (SEQ ID NO: 36): |
| | (108162-110408) | CGCAAAACAGAGGGTTCGAAGGAA |
| | | AACAGGAAACCTCTACGCATAGGC |
| | | CACTAGTGGATC |
| SEC4 | NC_001138 | F (SEQ ID NO: 37): |
| | (130329-130976) | TAGTGGGAGCGGAAACAGTTCTAA |
| | | ATCAAATTGCTGTTGAACGCTGCA |
| | | GGTCGACAACCC |
| | | R (SEQ ID NO: 38): |
| | | TTCACGATTAATTCTCAAAGAAGC |
| | | AAAAATCTTCTTTTCTGCATAGGC |
| | | CACTAGTGGATC |
| SPS19 | NC_001146 | F (SEQ ID NO: 39): |
| | (259579-260457) | TCCAGAAGCCTTAATAAAGAGTAT |
| | | GACATCTAAATTATAAACGCTGCA |
| | | GGTCGACAACCC |
| | | R (SEQ ID NO: 40): |
| | | GAAAGTGTCATATAATAATAGTAC |
| | | TCAATACCTATACGTAGCATAGGC |
| | | CACTAGTGGATC |
| TES1 | NC_001142 | F (SEQ ID NO: 41): |
| | (468196-467147) | TGTCTACGGGTCAGAACGAGACAT |
| | | TCGAGCCAAGTTCTGAACGCTGCA |
| | | GGTCGACAACCC |
| | | R (SEQ ID NO: 42): |
| | | AATATATATGTATGTGTTTATACG |
| | | TGGGAGGGAATTGTCCGCATAGGC |
| | | CACTAGTGGATC |
| YOR084W | NC_001147 | F (SEQ ID NO: 43): |
| | (480589-481752) | GAATGAAGCTTTGGTTAAAACGAC |
| | | TAAACAAAAACTGTAAACGCTGCA |
| | | GGTCGACAACCC |
| | | R (SEQ ID NO: 44): |
| | | ATTATTATTGAATAATATATGTAA |
| | | TAGTGTACACAAGTGTGCATAGGC |
| | | CACTAGTGGATC |
| PEX13 | NC_001144 | F (SEQ ID NO: 106): |
| | (537274-538434) | GAAAATTGAGCATGTTGATGATGA |
| | | AACGCGTACACACTAGACGCTGCA |
| | | GGTCGACAACCC |
| | | R (SEQ ID NO: 107): |
| | | TATATATATATGCGAATATATGTG |
| | | TGCAAATATTGATGCAGCATAGGC |
| | | CACTAGTGGATC |
| PEX11 | NC_001147 | F (SEQ ID NO: 108): |
| | (47932-48642) | ATCTATCCTTGGTATGCAAGACAT |
| | | GTGGAAAGCTACATAGACGCTGCA |
| | | GGTCGACAACCC |
| PEX11 | | R (SEQ ID NO: 109): |
| | | TCAAACATAAGCGGAGAATAGCCA |
| | | AATAAAAAAAAAAGATGAAAAGAA |
| | | AGGCATAGGCCACTAGTGGATC |
| AAT2 | NC_001144 | F (SEQ ID NO: 110): |
| | (196830-198086) | TGAAGTGGTGCGCTTCTATACTAT |
| | | TGAAGCTAAATTGTAAACGCTGCA |
| | | GGTCGACAACCC |
| | | R (SEQ ID NO: 111): |
| | | ATGAAGAGTGTAATAGGTAAGTAT |
| | | AAGTATTATTTAATCAGCATAGGC |
| | | CACTAGTGGATC |
| GPD1 | NC_001136 | F (SEQ ID NO: 112): |
| | (411822-412997) | GCCGGACATGATTGAAGAATTAGA |
| | | TCTACATGAAGATTAGACGCTGCA |
| | | GGTCGACAACCC |
| | | R (SEQ ID NO: 113): |
| | | AGTGGGGGAAAGTATGATATGTTA |
| | | TCTTTCTCCAATAAATGCATAGGC |
| | | CACTAGTGGATC |
| ANT1 | NC_001139 | F (SEQ ID NO: 114): |
| | (469097-472303) | CCTAAAGCACAACGGACAACGCAA |
| | | GCTGGCTTCCACTTGAACGCTGCA |
| | | GGTCGACAACCC |
| | | R (SEQ ID NO: 115): |
| | | TCTAAACGCAATGTGCTTATTTCA |
| | | GTAATAGTAAGGATTCGCATAGGC |
| | | CACTAGTGGATC |
| CAT2 | NC_001145 | F (SEQ ID NO: 116): |
| | (192788-194800) | CGCCTTGGAAAATGAGAATAAACG |
| | | AAAAGCAAAGTTATGAACGCTGCA |
| | | GGTCGACAACCC |
| | | R (SEQ ID NO: 117): |
| | | AAAATATTCACAAATTAATTGAAG |
| | | AGGAAAGGTGAAAAATGCATAGGC |
| | | CACTAGTGGATC |
| CIT2 | NC_001135 | F (SEQ ID NO: 118): |
| | (120944-122326) | ATACAAGGAATTGGTCAAAAACAT |
| | | TGAAAGCAAACTATAGACGCTGCA |
| | | GGTCGACAACCC |
| | | R (SEQ ID NO: 119): |
| | | AAAAATATGCAGAGGGGTGTAAAA |
| | | GTAGGATGTAATCCAAGCATAGGC |
| | | CACTAGTGGATC |
| CTA1 | NC_001136 | F (SEQ ID NO: 120): |
| | (968129-969676) | AAAACATGCTTCTGAGCTTTCGAG |
| | | TAACTCCAAATTTTGAACGCTGCA |
| | | GGTCGACAACCC |
| | | R (SEQ ID NO: 121): |
| | | TGTCGTGGAAACAACGCCACTCAT |
| | | TTGTTACTTGAGCGTTGCATAGGC |
| | | CACTAGTGGATC |
| ECI1 | NC_001144 | F (SEQ ID NO: 122): |
| | (706200-707042) | TAGGCAGCTGGGCTCGAAACAAAG |
| | | GAAGCATCGTTTATGAACGCTGCA |
| | | GGTCGACAACCC |
| | | R (SEQ ID NO: 123) |
| | | TATATTGTGTGTGCGTTTTGTTTC |
| | | ACTGAGAAAGCGGACGGCATAGGC |
| | | CACTAGTGGATC |
| FAA2 | NC_001137 | F (SEQ ID NO: 124): |
| | (184540-186774) | ATACGCCGAAGGTTCACTAGTCAA |
| | | GACAGAAAAGCTTTAGACGCTGCA |
| | | GGTCGACAACCC |
| | | R (SEQ ID NO: 125): |
| | | TTTTTTCTAGTTTGAATGTGTTCC |
| | | AAATCGTCATAAGTACGCATAGGC |
| | | CACTAGTGGATC |
| FAT1 | NC_001134 | F (SEQ ID NO: 126): |
| | (318266-320275) | TGATTGGGAAGCCATCGATGCACA |
| | | AACAATTAAATTATGAACGCTGCA |
| | | GGTCGACAACCC |
| FAT1 | | R (SEQ ID NO: 127): |
| | | TGCAAGGAAAAATACTTTATCCTA |
| | | ATTCAGGAACATCAAAGCATAGGC |
| | | CACTAGTGGATC |
| INP1 | NC_001145 | F (SEQ ID NO: 128): |
| | (670062-671324) | ATTTCAGAGGAGATCCATATCTGG |
| | | TCTTGGCGACCTTTGAACGCTGCA |
| | | GGTCGACAACCC |
| | | R (SEQ ID NO: 129): |
| | | ACACCTACATTCATTTGTGCAGTT |
| | | ATGCTTTGAACTTCATGCATAGGC |
| | | CACTAGTGGATC |
| INP2 | NC_001145 | F (SEQ ID NO: 130): |
| | (584270-586387) | TTTGTATGAATTAAAAGGATTACT |
| | | AGGAAATGATTCATGAACGCTGCA |
| | | GGTCGACAACCC |
| | | R (SEQ ID NO: 131): |
| | | GTGTAATTAGTTATTTCAAAGTAC |
| | | ATATTAAAATATATTAGCATAGGC |
| | | CACTAGTGGATC |
| MDH3 | NC_001136 | F (SEQ ID NO: 132): |
| | (315357-316388) | AAAAGGCAAGAGTTTCATCCTAGA |
| | | CTCTTCCAAGCTATGAACGCTGCA |
| | | GGTCGACAACCC |
| | | R (SEQ ID NO: 133): |
| | | AGGAGTATAGAGTTAAGAAAAATA |
| | | TAAAAATTGAAGTAGCGCATAGGC |
| | | CACTAGTGGATC |
| NPY1 | NC_001139 | F (SEQ ID NO: 134): |
| | (376104-377258) | CTATAAAAACTTACGTAAGACCTC |
| | | ATCGAGCCATCTATAGACGCTGCA |
| | | GGTCGACAACCC |
| | | R (SEQ ID NO: 135): |
| | | CTGAAGCACGCCTATTTATCAATG |
| | | TTTATTATATTAAAAAGCATAGGC |
| | | CACTAGTGGATC |
| PCD1 | NC_001144 | F (SEQ ID NO: 136): |
| | (441716-442738) | CTACATGAAGCACCTGCTGGAGTG |
| | | CCGCTCGCTTTGGTAAACGCTGCA |
| | | GGTCGACAACCC |
| | | R (SEQ ID NO: 137): |
| | | TGAGAGTATTGTTAGGCAACGCAT |
| | | TATACCACAGTTTTTTGCATAGGC |
| | | CACTAGTGGATC |
| PEX2 | NC_001142 | F (SEQ ID NO: 138): |
| | (36919-37734) | TGGATCCTCTGGGAGACTGACCGC |
| | | CTCACCAGTGTACTAAACGCTGCA |
| | | GGTCGACAACCC |
| | | R (SEQ ID NO: 139): |
| | | ATACACATATATAGAGATACAAGC |
| | | GAGGGAACGGGGCCCTGCATAGGC |
| | | CACTAGTGGATC |
| PEX4 | NC_001139 | F (SEQ ID NO: 140): |
| | (756901-757452) | GTACTTCCTAGCAGAAAGAGAGCG |
| | | GATCAACAACCATTGAACGCTGCA |
| | | GGTCGACAACCC |
| | | R (SEQ ID NO: 141): |
| | | CCCATTGTTTGCCATTCGAACACA |
| | | TCCATCCTACGTGGTAGCATAGGC |
| | | CACTAGTGGATC |
| PEX6 | NC_001146 | F (SEQ ID NO: 142): |
| | (19541-22633) | TCATTATGAAGCGGTGAGAGCTAA |
| | | TTTTGAAGGTGCTTAAACGCTGCA |
| | | GGTCGACAACCC |
| | | R (SEQ ID NO: 143): |
| | | ATATTTACAAATTTACCTATACGC |
| | | TCTGAGTTGATATTACGCATAGGC |
| | | CACTAGTGGATC |
| PEX7 | NC_001136 | F (SEQ ID NO: 144): |
| | (740470-741597) | ATGGGATGGAAATTTATTTGTATG |
| | | GAACGGCTTAGGTTGAACGCTGCA |
| | | GGTCGACAACCC |
| PEX7 | | R (SEQ ID NO: 145): |
| | | GTTTAAATAATGCAAAAAATTTGT |
| | | GTAAAAAGAATATGTGGCATAGGC |
| | | CACTAGTGGATC |
| PEX8 | NC_001139 | F (SEQ ID NO: 146): |
| | (637748-639517) | GTACACAACGGTCTTATCAAGTCA |
| | | ATCTTCTAAATTATAGACGCTGCA |
| | | GGTCGACAACCC |
| | | R (SEQ ID NO: 147): |
| | | AGGAATATAAAAAGGCGCTACTAT |
| | | AAAGTACTTAATGATAGCATAGGC |
| | | CACTAGTGGATC |
| PEX10 | NC_001136 | F (SEQ ID NO: 148): |
| | (998860-999873) | ACACTGTCAACCACAGGAAATTCT |
| | | GGTCCTGCGGCAATAGACGCTGCA |
| | | GGTCGACAACCC |
| | | R (SEQ ID NO: 149): |
| | | GACAATGCTAAAAGAGTAGTCAAA |
| | | TTATTGATTAGTTCCTGCATAGGC |
| | | CACTAGTGGATC |
| PEX12 | NC_001145 | F (SEQ ID NO: 150): |
| | (324235-325434) | ATGGGAAGTTGTGACAGGTATTAG |
| | | GAAGCTACTAATCTGAACGCTGCA |
| | | GGTCGACAACCC |
| | | R (SEQ ID NO: 151): |
| | | TATATATTACACAGAATTATTTTC |
| | | TTCACTTCCTCCGTCAGCATAGGC |
| | | CACTAGTGGATC |
| PEX17 | NC_001146 | F (SEQ ID NO: 152): |
| | (245618-246217) | AATCAAAGGTTGGTTTGTGAATGG |
| | | CCAAGTGCCAAGGTAAACGCTGCA |
| | | GGTCGACAACCC |
| | | R (SEQ ID NO: 153): |
| | | CACTAGAGCGTTTTAAATTCAATG |
| | | CTATTATTTTTGATTGGCATAGGC |
| | | CACTAGTGGATC |
| PEX22 | NC_001133 | F (SEQ ID NO: 154): |
| | (42178-42720) | CGATGTAGAGGATGTGCTGATTGA |
| | | CACTTTATGCAATTAAACGCTGCA |
| | | GGTCGACAACCC |
| | | R (SEQ ID NO: 155): |
| | | TTTATTCTTTACATACTGTTACAA |
| | | GAAACTCTTTTCTACAGCATAGGC |
| | | CACTAGTGGATC |
| PEX25 | NC_001148 | F (SEQ ID NO: 156): |
| | (337435-338619) | GATAACAACAAAGAGGTCACTTTG |
| | | CTCTTCAAAAGATTGAACGCTGCA |
| | | GGTCGACAACCC |
| | | R (SEQ ID NO: 157): |
| | | TATATATGTACATATCTATATGTA |
| | | TACATATTTTTATATAGCATAGGC |
| | | CACTAGTGGATC |
| PEX27 | NC_001147 | F (SEQ ID NO: 158): |
| | (710447-711577) | CAAAGTCACTTCGGCTAATGAACA |
| | | TACAAGCGCTGTTTGAACGCTGCA |
| | | GGTCGACAACCC |
| | | R (SEQ ID NO: 159): |
| | | ACGAAATAAAGAGGGATGCAACGA |
| | | ACTTGGTCATCTGTTGGCATAGGC |
| | | CACTAGTGGATC |
| PEX29 | NC_001136 | F (SEQ ID NO: 160): |
| | (1415202-1416866) | AATCGAAGAGCTAACAGACACTCT |
| | | CAATTCAACTATATAAACGCTGCA |
| | | GGTCGACAACCC |
| | | R (SEQ ID NO: 161): |
| | | GACTGTATCATCAGTGAACATATA |
| | | GTATAACAAATCAAGTGCATAGGC |
| | | CACTAGTGGATC |
| PEX30 | NC_001144 | F (SEQ ID NO: 162): |
| | (779215-780786) | AAATCCAACCATTGGTCGCGATAG |
| | | CAAGAAGGCCGTATGAACGCTGCA |
| | | GGTCGACAACCC |
| PEX30 | | R (SEQ ID NO: 163): |
| | | TAGAGATTATATTATGTAAAGGTA |
| | | AAAACGGGAGCGAGCAGCATAGGC |
| | | CACTAGTGGATC |
| PEX31 | NC_001139 | F (SEQ ID NO: 164): |
| | (502942-504330) | AATACAAATATCTGATGTTTCAAT |
| | | GTCTCCTTCTCTATAAACGCTGCA |
| | | GGTCGACAACCC |
| | | R (SEQ ID NO: 165): |
| | | ACCAGTGTGAACGTTGTTGTCCAT |
| | | ATGGGGCATGCACTCAGCATAGGC |
| | | CACTAGTGGATC |
| PEX32 | NC_001134 | F (SEQ ID NO: 166): |
| | (572366-573607) | CAGGTCAAGAAAATGGAAACGACG |
| | | CCTCTTCCATTTGTAAACGCTGCA |
| | | GGTCGACAACCC |
| | | R (SEQ ID NO: 167): |
| | | ACCAACTATATATGCAGTTTAGAG |
| | | GCTTAAAGCAATACTAGCATAGGC |
| | | CACTAGTGGATC |
| PXA1 | NC_001148 | F (SEQ ID NO: 168): |
| | (273254-275866) | TGAGAGGACGAAGCTACGGGAAAA |
| | | GCTTGAAATTATTTGAACGCTGCA |
| | | GGTCGACAACCC |
| | | R (SEQ ID NO: 169): |
| | | TATATTCGCTAAATAAAATCTCTC |
| | | CCTTTCTAGGGTGTTTGCATAGGC |
| | | CACTAGTGGATC |
| PXA2 | NC_001143 | F (SEQ ID NO: 170): |
| | (86230-88791) | AAAAGTTAAAACAAAAAAGGAAGA |
| | | AGGAAAGGAGAGGTAAACGCTGCA |
| | | GGTCGACAACCC |
| | | R (SEQ ID NO: 171): |
| | | CAATTTATACATGATTTGGATCCT |
| | | CCTTTGGCTATGTATGGCATAGGC |
| | | CACTAGTGGATC |

Table 1: Provided are mRNA names along with their nucleic acid sequences (GenBank Accession numbers of contigs with the specific position of each of the mRNA nucleic acid sequences) and the forward (F) and reverse (R) primers used to prepare an mRNA-specific integration cassette for homologous recombination within the yeast genome. The underlined sequences in the forward or reverse primers correspond to the nucleic acid sequence derived from the plasmid upstream (5′) to thefirst loxP site (in the forward primer) or to the plasmid sequence downstream (3′) to the viral MS2L (in the reverse primer) of the integration cassette.

TABLE 3
Primers used for preparation of constructs

| | Primer sequence |
|---|---|
| Primer name | (SEQ ID NO: ) (5′-3′) |
| pSL-MS2-12X mut.F | 5′-CGTCGACCTGAGGTGATATCAACCCGGGC |
| | CC-3′ |
| | (SEQ ID NO: 45) |
| pSL-MS2-12X mut. R | 5′-GGGCCCGGGTTGATATCACCTCAGGTCGA |
| | CG-3′ |
| | (SEQ ID NO: 46) |
| GFP-RV F | 5′-GATATCGTGTCTAAAGGTGAAGAATTATT |
| | C-3′ |
| | (SEQ ID NO: 47) |
| GFP-RV R | 5′-GATATCTTTGTACAATTCATCCATAC |
| | C-3′ |
| | (SEQ ID NO: 48) |
| pMS2CPGFP(X2) | 5′-GAATTGTACAAAGAGATCAAGCTTATC |
| mut. F | G-3′ |
| | (SEQ ID NO: 49) |
| pMS2CPGFP(X2) | 5′-CGATAAGCTTGATCTCTTTGTACAATT |
| mut. | C-3′ |
| | (SEQ ID NO: 50) |
| OX1A-Orf F | 5′-TGACTGGTCGACCGATGTTCAAACTCACC |
| | TC-3′ |
| | (SEQ ID NO: 51) |
| OXA1-Orf R | 5′-TGACTGCCCGGGTTTTTTGTTATTAATGA |
| | AG-3′ |
| | (SEQ ID NO: 52) |
| OX1A-Utr F | 5′-AGCGAGCTCTAAAGGCTCTATATCTCT |
| | C-3′ |
| | (SEQ ID NO: 53) |
| OXA1-Utr R | 5′-TGACTGGAGCTCATCGCAAGGCTGTTTTA |
| | AG-3′ |
| | (SEQ ID NO: 54) |
| mRFP F | 5′-TCAGTCACCCGGGGCCTCCTCCGAGGAC |
| | G-3′ |
| | (SEQ ID NO: 55) |
| mRFP R | 5′-TCAGTCGAGCTCTTAGGCGCCGGTGG |
| | A-3′ |
| | (SEQ ID NO: 56) |
| mRFP HindIII F | 5′-GACGATGAAAGCTTAGGCGCCGGTGCCTC |
| | CTCCGAGGACGTCATC-3′ |
| | (SEQ ID NO: 57) |
| mRFP HindIII R | 5′-CTCAGCTTCCTTTCGGGCTTTGTTAG |
| | C-3′ |
| | (SEQ ID NO: 58) |
| ASH1-Det F | 5′-CTGCGAAATTGAAGGGTACCG-3′ |
| | (SEQ ID NO: 59) |
| ASH1-Det R | 5′-GCACAGACAAGGAGAGAAATG-3′ |
| | (SEQ ID NO: 60) |
| SRO7-Det F | 5′-CGACTATGCTACCGCCATGGG-3′ |
| | (SEQ ID NO: 61) |
| SRO7-Det R | 5′-CAAACCTTCGTAAAACTAGACATGTATAA |
| | TG-3′ |
| | (SEQ ID NO: 62) |
| OXA1-Det F | 5′-TGACTGGTCGACCGATGTTCAAACTCACC |
| | TC-3′ |
| | (SEQ ID NO: 63) |
| OXA1-Det R | 5′-TGACTGGAGCTCATCGCAAGGCTGTTTTA |
| | AG-3′ |
| | (SEQ ID NO: 64) |
| PEX3-Det F | 5′-GAACGAATACCTGGCCACTC-3′ |
| | (SEQ ID NO: 65) |
| PEX3-Det R | 5′-TCAGTCAGTGAGCTCCCGAACATTGGGCA |
| | C-3′ |
| | (SEQ ID NO: 66) |
| SNC1-Det F | 5′-GAAAAGCCATGTGGTACAAGG-3′ |
| | (SEQ ID NO: 67) |
| SNC1-Det R | 5′-ATAAGAACAAAGTAAATATACGCCC-3′ |
| | (SEQ ID NO: 68) |
| POX1-Det F | 5′-CATAAGATGGCCTCTCACTAGG-3′ |
| | (SEQ ID NO: 69) |
| POX1-Det R | 5′-CCGTATCAGTTTTCAATATAGGATCA-3′ |
| | (SEQ ID NO: 70) |
| POT1-Det F | 5′-CCATCCCTTGGGTTGTACTG-3′ |
| | (SEQ ID NO: 71) |
| POT1-Det R | 5′-TTCAAATCAGCCCTCAAAGG-3′ |
| | (SEQ ID NO: 72) |
| PEX14-Det F | 5′-ATGACCCGGGATGAGTGACGTGGTCAGT |
| | A-3′ |
| | (SEQ ID NO: 73) |
| PEX14-Det R | 5′-TCGGAGCTCACAATATCTAGAGCCTC-3′ |
| | (SEQ ID NO: 74) |
| TES1-Det F | 5′-ATGACCCGGGATGAGTGCTTCCAAAATGG |
| | C-3′ |
| | (SEQ ID NO: 75) |
| TES1-Det R | 5′-GATACCCGCTCGTGAAAGG-3′ |
| | (SEQ ID NO: 76) |
| SPS19-Det F | 5′-TCCCCGGGATGGATACTATGAATACAGCA |
| | A-3′ |
| | (SEQ ID NO: 77) |
| SPS19-Det R | 5′-TGGAGCTCCTTAGTTCAAACATATGGT |
| | G-3′ |
| | (SEQ ID NO: 78) |
| PEX11-Det F | 5′-GGCGATGAGCATGAGGATCAC-3′ |
| | (SEQ ID NO: 79) |
| PEX11-Det R | 5′-GAAGGGTCGAATCAAACATAA-3′ |
| | (SEQ ID NO: 80) |
| PEX12-Det F | 5′-GAGGCCTGTCCCGTTTGCG-3′ |
| | (SEQ ID NO: 81) |
| PEX12-Det R | 5′-CAATGGGAAATTTCAAATATG-3′ |
| | (SEQ ID NO: 82) |
| PEX13-Det F | 5′-GTTCCAGAAAACCCAGAGATG-3′ |
| | (SEQ ID NO: 83) |
| PEX13-Det R | 5′-GTTTCTGCTGATTCTCCCTGG-3′ |
| | (SEQ ID NO: 84) |
| INP1-Det F | 5′-CCGATGCCGTGTCAATCTCC-3′ |
| | (SEQ ID NO: 85) |
| INP1-Det R | 5′-TTGAGCTCCAATTTGAAACTGCTGGTA |
| | A-3′ |
| | (SEQ ID NO: 86) |
| NPY1-Det F | 5′-CCGATGCCGTGTCAATCTCC-3′ |
| | (SEQ ID NO: 87) |
| NPY1-Det R | 5′-GTTTTCTTCGGGTAACTGAGTG-3′ |
| | (SEQ ID NO: 88) |
| PCD1-Det F | 5′-CAACCGAACGGAAGAAGTG-3′ |
| | (SEQ ID NO: 89) |
| PCD1-Det R | 5′-GATTAAGGACATCCAGTATG-3′ |
| | (SEQ ID NO: 90) |
| CIT2-Det F | 5′-TAGCACCTGGCGTATTGACT-3′ |
| | (SEQ ID NO: 172) |
| CIT2-Det R | 5′-CGAGGAAGGAAATAGTAACG-3′ |
| | (SEQ ID NO: 173) |
| IDP1-Det F | 5′-GCCTCAATATTTGCCTGGAC-3′ |
| | (SEQ ID NO: 174) |
| IDP1-Det R | 5′-TGGATCTCTCCTGCCTAATC-3′ |
| | (SEQ ID NO: 175) |
| PEX10-Det F | 5′-CAATTACTAGGTCGTCTGTTGGTC-3′ |
| | (SEQ ID NO: 176) |
| PEX10-Det R | 5′-CCACATTGGTGTATAGTTGG-3′ |
| | (SEQ ID NO: 177) |
| PEX17-Det F | 5′-GTCCACTCAAACTTCAGACG-3′ |
| | (SEQ ID NO: 178) |
| PEX17-Det R | 5′-GTTTCTGCACTTTCACTTGC-3′ |
| | (SEQ ID NO: 179) |
| PEX2-Det F | 5′-ATTCGCTGGGTTAGAATACC-3′ |
| | (SEQ ID NO: 180) |
| PEX2-Det R | 5′-CGTCGCTTCCCACATCGTCC-3′ |
| | (SEQ ID NO: 181) |
| PEX22-Det F | 5′-AACAAGCCATTGGGGATGCC-3′ |
| | (SEQ ID NO: 182) |
| PEX22-Det R | 5′-CCCTGGCATTGTTAGACATC-3′ |
| | (SEQ ID NO: 183) |
| PEX25-Det F | 5′-TGTTAATAACGACGCAGAGG-3′ |
| | (SEQ ID NO: 184) |
| PEX25-Det R | 5′-GGAATGACTACCGCCACCCC-3′ |
| | (SEQ ID NO: 185) |
| PEX27-Det F | 5′-CAGTGGTAATGCAATAAAGG-3′ |
| | (SEQ ID NO: 186) |
| PEX27-Det R | 5′-TCAAGTGGAAGCGGAGTGGG-3′ |
| | (SEQ ID NO: 187) |
| PEX29-Det F | 5′-AAACCTTGGTGAGGAGGAAG-3′ |
| | (SEQ ID NO: 188) |
| PEX29-Det R | 5′-TAGTACCAGCAGCGGGAAGG-3′ |
| | (SEQ ID NO: 189) |
| PEX30-Det F | 5′-CACGAATGGTTTAACCGCTG-3′ |
| | (SEQ ID NO: 190) |
| PEX30-Det R | 5′-GAATACTTTCCCATCCGC-3′ |
| | (SEQ ID NO: 191) |
| PEX32-Det F | 5′-GAAGGGTGATGACCACATTC-3′ |
| | (SEQ ID NO: 192) |
| PEX32-Det R | 5′-TCTATTTGGATTGTTCCCTC-3′ |
| | (SEQ ID NO: 193) |
| PEX6-Det F | 5′-TCTCTGCTCAGATGCAATGC-3′ |
| | (SEQ ID NO: 194) |
| PEX6-Det R | 5′-ATACTATGAGCCGGGGAGGG-3′ |
| | (SEQ ID NO: 195) |
| PEX7-Det F | 5′-ATGCGCACGGGCTGGCAATC-3′ |
| | (SEQ ID NO: 196) |
| PEX7-Det R | 5′-GTCAGAAGCGTTGTTACCC-3′ |
| | (SEQ ID NO: 197) |
| PEX8-Det F | 5′-GGGACTCTTTGCACGAGACG-3′ |
| | (SEQ ID NO: 198) |
| PEX8-Det R | 5′-TTGAAGGGGGGTATCTTTGG-3′ |
| | (SEQ ID NO: 199) |
| PXA1-Det F | 5′-GGTGGTGAAAAGCAAAGAGT-3′ |
| | (SEQ ID NO: 200) |
| PXA1-Det R | 5′-AGGTTGTCCTTGATACGTGG-3′ |
| | (SEQ ID NO: 201) |
| PXA2-Det F | 5′-GATCAACAGGTGCCACTTTG-3′ |
| | (SEQ ID NO: 202) |
| PXA2-Det R | 5′-CAGTGGGTCGTGACATGAAT-3′ |
| | (SEQ ID NO: 203) |

Table 2: Provided are primers sequences used for the plasmid construction: SEQ ID NOs: 45 and 46 are primers used to add a EcoRV site to the MS2L sequence in pSL-MS2-12X plasmid. SEQ ID NOs: 47 and 48 were used for GFP amplification from pCP-GFP. SEQ ID NOs: 49 and 50 were used to eliminate the 3′ EcoRV site in pMS2CPGFP(x2). SEQ ID NOs: 51 and 52 and SEQ ID NOs: 53 and 54 were used to amplify the OXA1 ORF and 3′-UTR, respectively, from genomic DNA. SEQ IDNOs: 55 and 56 are used for mRFP amplification to aid in the construction of pAD4Δ-OXA1-RFP, SEQ ID NOs: 57 and 58 were used for mRFP amplification for the construction of pRFPLOXHIS5MS2L. SEQ ID NOs: 59-90 are used for the detection of integration for each tagged gene (one oligonucleotide pair was used for each gene of interest).

TABLE 3
Yeast strains used in the present study

| Strain | Genotype | Source |
|---|---|---|
| BY4741 | MATa his3Δ1 leu2Δ0 met15Δ0 ura3Δ0 | Euroscarf |
| W303-1a | MATa ade2 can1 his3 leu2 lys2 trp1 ura3 | J. Hirsch |
| LHY1 (ASH1_{INT}) | MATa his3Δ1 leu2Δ0 met15ΔO ASH1::loxP::MS2L::ASH1^{3′-UTR} | This study |
| LHY2 (SRO7_{INT}) | MATa ade2 can1 his3 leu2 lys2 trp1 SRO7::loxP::MS2L::SRO7^{3′UTR} | This study |
| LHY3 (OXA1_{INT}) | MATa his3Δ1 leu2Δ0 met15Δ0 ura3Δ0 OXA1::loxP::MS2L::OXA1^{3′-UTR} | This study |
| LHY4 (SNC1_{INT}) | MATa his3Δ1 leu2Δ0 met15Δ0 ura3Δ0 SNC1::loxP::MS2L::SNC1^{3′-UTR} | This study |
| LHY5 (PEX3_{INT}) | MATa his3Δ1 leu2Δ0 met15Δ0 ura3Δ0 PEX3::loxP::MS2L::PEX3^{3′-UTR} | This study |
| LHY6 (POX1_{INT}) | MATa his3Δ1 leu2Δ0 met15Δ0 ura3Δ0 POX1::loxP::MS2L:: POX1^{3′-UTR} | This study |
| LHY7 (POT1_{INT}) | MATa his3Δ1 leu2Δ0 met15Δ0 ura3Δ0 POT1::loxP::MS2L:: POT1^{3′-UTR} | This study |
| LHY8 (PEX14_{INT}) | MATa his3Δ1 leu2Δ0 met15Δ0 ura3Δ0 PEX14::loxP::MS2L::PEX14^{3′-UTR} | This study |
| LHY9 (TES1_{INT}) | MATa his3Δ1 leu2Δ0 met15Δ0 ura3Δ0 TES1::loxP::MS2L::TES1^{3′-UTR} | This study |
| LHY10 (SPS19_{INT}) | MATa his3Δ1 leu2Δ0 met15Δ0 ura3Δ0 SPS19::loxP::MS2L::SPS19^{3′-UTR} | This study |
| LHY11 (PEX11_{INT}) | MATa his3Δ1 leu2Δ0 met15Δ0 ura3Δ0 PEX11::loxP::MS2L::PEX11^{3′-UTRK} | This study |
| LHY12 (PEX12_{INT}) | MATa his3Δ1 leu2Δ0 met15Δ0 ura3Δ0 PEX12::loxP::MS2L::PEX12^{3′-UTR} | This study |
| LHY13 (PEX13_{INT}) | MATa his3Δ1 leu2Δ1 met15Δ0 ura3Δ0 PEX13::loxP::MS2L::PEX13^{3′-UTR} | This study |
| LHY14 (INP1_{INT}) | MATa his3Δ1 leu2Δ0 met15Δ0 ura3Δ0 INP1::loxP::MS2L::INP1^{3′-UTR} | This study |
| LHY15 (NPY1_{INT}) | MATa his3Δ1 leu2Δ0 met15Δ0 ura3Δ0 NPY1::loxP::MS2L::NPY1^{3′-UTR} | This study |
| LHY16 (PCD1_{INT}) | MATa his3Δ1 leu2Δ0 met15Δ0 ura3Δ0 PCD1::loxP::MS2L::PCD1^{3′-UTR} | This study |
| LHY1P | MATa/α his3Δ1/his3Δ1 leu2Δ0/leu2Δ0 met15Δ0/MET15 ura3Δ0/ura3Δ0 | This study |
| | ASH1/ASH1::mRFP::loxP::MS2L::ASH1^{3′-UTR} | |

Table 3: These yeast strains were used for the detection of endogenous mRNAs in vivo using MS2-CP-GFP(x2) and MS2-CP-GFP(x3).

TABLE 4
Primers used for the amplification of the
mRFP-loxP-MS2L protein-mRNA localization cassette
as well as for the amplification of the mRFP-loxP
protein localization cassette

| Protein | |
|---|---|
| (GenBank | Primer sequence |
| Accession No.) | (SEQ ID NO: ) (5′→3′) |
| Ash1 | F (SEQ ID NO: 91) |
| (NP_012736) | CTTATTTTGTAATTACATAACTGAGACAGTAGAGAA |
| | TGGAGGCGCCGGTGCCTCCTCCG |
| | R (SEQ ID NO: 2) |
| | ATGTCTCTTATTAGTTGAAAGAGATTCAGTTATCCA |
| | TGTAGCATAGGCCACTAGTGGATC |
| Sro7 | F (SEQ ID NO: 93) |
| (NP_015357) | GCAGACTGGAAAAGATGTAATGAAAGGTGCCCTTGG |
| | TTTTGGAGGCGCCGGTGCCTCCTCCG |
| | R (SEQ ID NO: 4) |
| | ATAGAAGGAAGTTGCTCATTACCCTGTATGAATTAG |
| | TGTATGTATCTGATATCGATCGCGCGCAG |
| Oxa1 | F (SEQ ID NO: 95) |
| (NP_011081) | CAAAATTGTTCACAAATCAAACTTCATTAATAACAA |
| | AAAAGGAGGCGCCGGTGCCTCCTCCG |
| | R (SEQ ID NO: 6) |
| | TTTATATTTTTATATTTACAGAGAGATATAGAGCCT |
| | TTATGCATAGGCCACTAGTGGATC |
| Pex3 | F (SEQ ID NO: 97) |
| (NP_010616) | CAGCAACTTTGGCGTCTCCAGCTCGTTTTCCTTCAA |
| | GCCTGGAGGCGCCGGTGCCTCCTCCG |
| | R (SEQ ID NO: 8) |
| | TATATATATTCTGGTGTGAGTGTCAGTACTTATTCA |
| | GAGAGCATAGGCCACTAGTGGATC |

Table 4: Provided are protein names along with their amino acid sequences (GenBank Accession numbers) and the forward (F) and reverse (R) primers used to prepare the mRFP-loxP-MS2L protein-mRNA localization cassette (pRFPLOXHIS5MS2L; SEQ ID NO: 96) and the mRFP-loxP protein localization cassette (pRFPLOXHIS5; SEQ ID NO: 105) for homologous recombination within the yeast genome. The underlined sequences in the forward or reverse primers correspond to thenucleic acid sequence derived from the red fluorescent protein (mRFP) sequence (in the forward primer) or the plasmid sequence (in the reverse primer) which is 3′ to MS2L of the mRFP-loxP-MS2L cassette or 3′ to the second (and more downstream) loxP site in the mRFP-loxP cassette.

### Example 1

### Generation of an mRNA Localization Construct for Directed Genomic Integration into the Gene-of-Interest

The Experimental Approach

To enable detection of intracellular localization of endogenous mRNAs in live cells, the present inventors have constructed an integration construct, as follows. The integration construct includes a yeast transformation selection marker flanked by loxP sites, for Cre-directed excision, upstream of 12 MS2 loop sequences. After integration, Cre-mediated excision of the selectable marker, and subsequent transcription from the endogenous promoter an mRNA sequence with a unique secondary structure is expressed for each gene of interest. Once expressed, the unique MS2L secondary structure can bind to a specific viral MS2 coat protein (MS2-CP) which is co-expressed in the cells. The inventors created an MS2-CP coat protein that is conjugated to either two or three tandem green fluorescent protein (GFP) proteins which, upon binding to the secondary structure of MS2L RNA, forms an intense and highly localized green fluorescence signal within living cells. The integration construct can be easily adapted to any yeast gene of interest by PCR amplification using specific primers. One of the PCR primers (the forward primer) includes the nucleic acid sequence derived from the 3′-end of the gene-of-interest conjugated to a nucleic acid sequence that corresponds to the sequence upstream (5′) of the first loxP site of the integration vector. The other primer (the reverse primer) includes a nucleic acid sequence derived from the 3′-UTR of the gene-of-interest conjugated to a nucleic acid sequence which corresponds to the sequence upstream (5′) to the MS2L loops in the integration construct. A schematic illustration of the integration construct is depicted is FIG. 1a.

The insertion cassette—The insertion cassette contains 12 MS2-CP binding sites (known as MS2 loops; MS2L) cloned downstream to the S. pombe HIS5 selectable marker which itself is flanked by loxP sites. The latter are used for Cre recombinase-mediated excision of the selectable marker after integration and upon cre heterologous expression in yeast (13). This step is necessary in order to place the MS2-CP binding sites directly downstream of the stop codon and upstream of the 3′-UTR, the latter being both important and often necessary for mRNA targeting in yeast and higher eukaryotes (3-7). Moreover, the present inventors have recently demonstrated that the 3′-UTR may facilitate the trafficking of a number of polarity and secretion factor mRNAs to the bud tip in vegetatively growing yeast and subsequent protein enrichment therein (14). Thus, integrity of the 3′-UTR within the transcript is likely to be essential for both proper mRNA and protein localization in yeast. As other genome-wide tagging strategies have employed integration constructs that invariably dissociate the 3′-UTR from the coding sequence, for example, upon insertion of the GFP gene and kanamycin resistance gene (a selectable marker) at the 3′-end of genes (11), it is likely that resulting mRNAs could be mislocalized. Given the importance of maintaining presence of 3′-UTR in the transcribed mRNA, and in close proximity to the coding region, excision of the selectable marker after integration is mandated (FIGS. 1a-c).

Experimental Results

Construction of an integration cassette for the ASH1 mRNA in yeast—Since both integration and Cre recombinase-mediated excision in yeast can be monitored by PCR (15), the tagging technique of the present invention was first tested on ASH1 mRNA (GenBank Accession No. NC_{—}001143, the nucleic acid sequence which begins at position 94504 and ends at position 96270). The ASH1 mRNA is known to be localized to the bud tip in yeast using both in situ and plasmid-based in vivo labeling methodologies (1, 2, 16, 17). During budding, ASH1 mRNA is actively exported from the mother cell and local translation in the daughter cell prevents mating-type switching (16-18).

Verification of integration of the ASH1-loxP cassette in yeast genome—PCR was used to amplify the loxP::SpHIS5::loxP::MS2L cassette (FIG. 1A) using a forward oligonucleotide complementary to the 3′ end of the coding region of ASH1 (including the stop codon) and a short sequence upstream of the first loxP site (SEQ ID NO:1), and a reverse oligonucleotide complementary to the beginning of the 3′-UTR of ASH1 and the end of the MS2 loop sequence (a non-repetitive stretch of nucleotides situated 3′ to the 12 MS2 loops) (SEQ ID NO:2). This 2213 bp fragment was used to transform wild-type yeast cells (BY4741) and upon selection in the absence of histidine led to the appearance of individual colonies on plates. DNA extraction from single colonies and amplification using a forward oligonucleotide complementary to the coding region of ASH1 and a reverse oligonucleotide complementary to the loxP::SpHIS5::loxP::MS2L cassette and the 3′-UTR of ASH1, revealed proper integration as evidenced by electrophoresis on ethidium bromide—stained agarose gels (FIG. 2a) and by DNA sequencing (data not shown). A frequency of up to ˜60% was typically observed for this ASH1::loxP::SpHIS5::loxP::MS2L::ASH1^{3′-UTR} integration event. Next, cre recombinase gene expression driven from a galactose-inducible promoter was used to excise SpHIS5 via the loxP sites. Recombination was verified by PCR analysis (FIG. 2b) and both marker excision and subsequent loss of histidine prototrophy were demonstrated to occur at a frequency of ˜100% to yield ASH1:loxP::MS2L::ASH1^{3′-UTR}. Finally, total RNA was extracted from yeast after Cre-mediated recombination and was subjected to reverse transcription (RT)-PCR followed by DNA sequencing which verified that both the MS2 loops and 3′-UTR were indeed present in the transcript (FIG. 2b and data not shown).

Altogether, these results demonstrate that a PCR-based strategy can be used to integrate viral RNA binding sites into the yeast genome with relative ease and efficiency.

### Example 2

### The MS2L-Integration Cassette Enables The Detection of mRNA Localization in Live Yeast Cells

Experimental Results

Localization of ASH1 mRNA after induction of the MS2-CP-GFP fusion protein requires at least 2 GFP tags—To visualize ASH1 mRNA localization in the ASH1::loxP::MS2L::ASH1^{3′-UTR} strain, an MS2-CP-GFP fusion protein was expressed under control of the MET25 methionine-repressible promoter. After a 1 hour of induction in medium lacking methionine, the cells were examined for the presence of fluorescent-labeled mRNA granules (granular mRNA) typically seen upon induction (1, 2, 19). While GFP fluorescence was detected, granular mRNA was not seen in these cells (FIG. 3a). Longer times of MS2-CP-GFP induction did not improve this result (data not shown). Because the endogenous levels of mRNA are on the order of 10-50-fold less than that expressed by plasmids (14), it was assumed that the lack of granular mRNA might be due to the low mRNA levels expressed from the native ASH1 promoter. To improve the signal, the present inventors have created double and triple GFP-tagged MS2-CP fusions and expressed them in ASH1::loxP::MS2L::ASH1^{3′-UTR} yeast. As is shown in FIGS. 3b-f, expression of the double GFP tag [GFP(X2)] led to the appearance of granules in 3% of the cells (n=200 cells), while expression of the triple GFP tag [GFP(x3)] led to the appearance of granules in 19% of the cells (n=200 cells). In the latter (FIGS. 3c-f), ASH1 mRNA granules were located at the bud tip in 98% of small- and medium-budded cells [S (FIG. 3d) and early G2-M (FIG. 3e) phase; n=91 cells] and at the bud neck in 82% of large budded cells [late G2-M phase (FIG. 3f); n=110 cells], as was seen in earlier studies (1, 2, 19). These granules were around 300-500 nm in size and were not stationary, but moved erratically in and around the bud tip, as seen previously using plasmid-based MS2-CP-GFP detection systems (1, 2, 14, 19). Thus, MS2-GFP(x2) and MS2-GFP(x3) labeling of endogenous ASH1 mRNA was identical to that observed using other detection systems.

In vivo localization of SRO7 mRNA to the bud tip in live yeast cells—To verify that other mRNAs can be localized using this novel integration strategy, the localization of SRO7 mRNA, which encodes a polarity and secretion factor involved in exocytosis (20), was examined. Previously, it was demonstrated by the present inventors that this mRNA is localized to the bud tip, as assayed using both in situ hybridization and plasmid-based MS2-GFP detection systems (14). Like ASH1 mRNA, SRO7 mRNA is delivered to the incipient bud in a manner dependent upon the SHE1-3 genes (14), which encode a type V myosin (She1/Myo4), an RNA binding protein (She2), and an adaptor protein (She3) (5). Moreover, both ASH1 and SRO7 mRNAs bind to She2 and are delivered to the bud along with cortical ER in an actin-dependent fashion (14). The localization of SRO7 mRNA in a SRO7::loxP::MS2L::SRO7^{3′-UTR} strain, created as described above, was examined by expressing MS2-GFP(x3). As is shown in FIGS. 4a-c, SRO7 mRNA is localized to the bud tip in at least 50% of small budded cells (n=100 cells), as previously seen using both in situ hybridization and plasmid-based MS2-CP-GFP systems (14). Thus, the MS2 loop genomic tagging strategy is also suitable for polarized mRNAs other than ASH1.

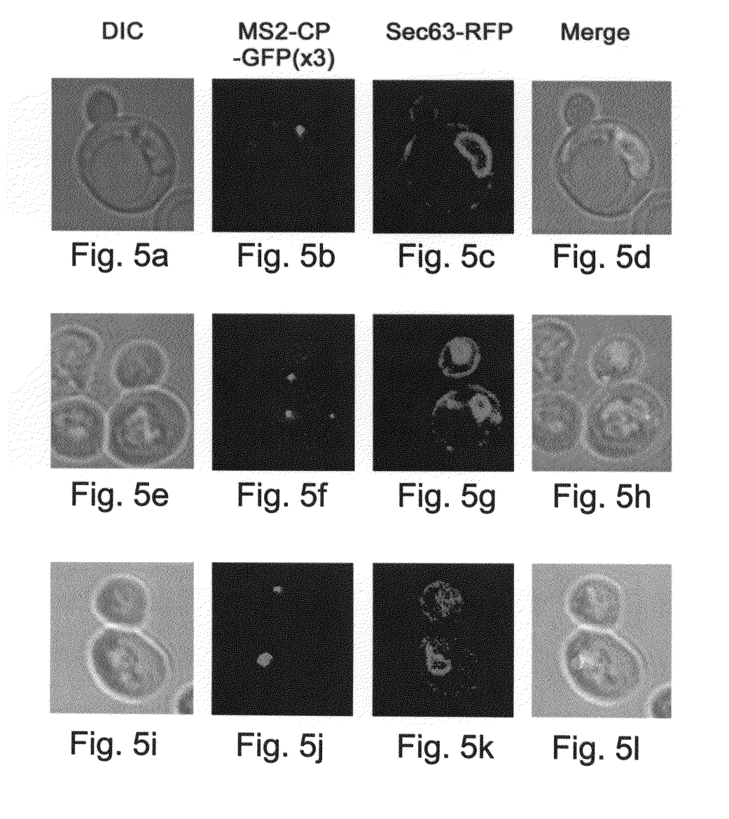

PEX3 mRNA localizes to the ER in live yeast cells—The localization of PEX3 mRNA, which encodes a peroxisomal protein that localizes to the endoplasmic reticulum (ER) upon translation and facilitates peroxisome assembly at the surface of the ER (21) was further examined. A strain including the PEX3::loxP::MS2L::PEX3^{3′-UTR} cassette was created and further examined for the localization of PEX3 mRNA in cells expressing MS2-GFP(x3). As is shown in FIGS. 5a-l, the fluorescent PEX3 mRNA granules were non-polarized (in contrast to ASH1 or SRO7 mRNAs) and localized to membranes labeled with Sec63-RFP, an endoplasmic reticulum (ER) marker. In addition, multiple fluorescent PEX3 mRNA granules could be observed in cells (through z sectioning) and were associated with Sec63-RFP, which yielded a typical ER labeling pattern. Additional studies in the lab have demonstrated that PEX3 mRNA co-fractionates with the ER (data not shown) and, thus, it was concluded by the present inventors that the mRNA encoding the peroxisomal assembly factor is ER-localized.

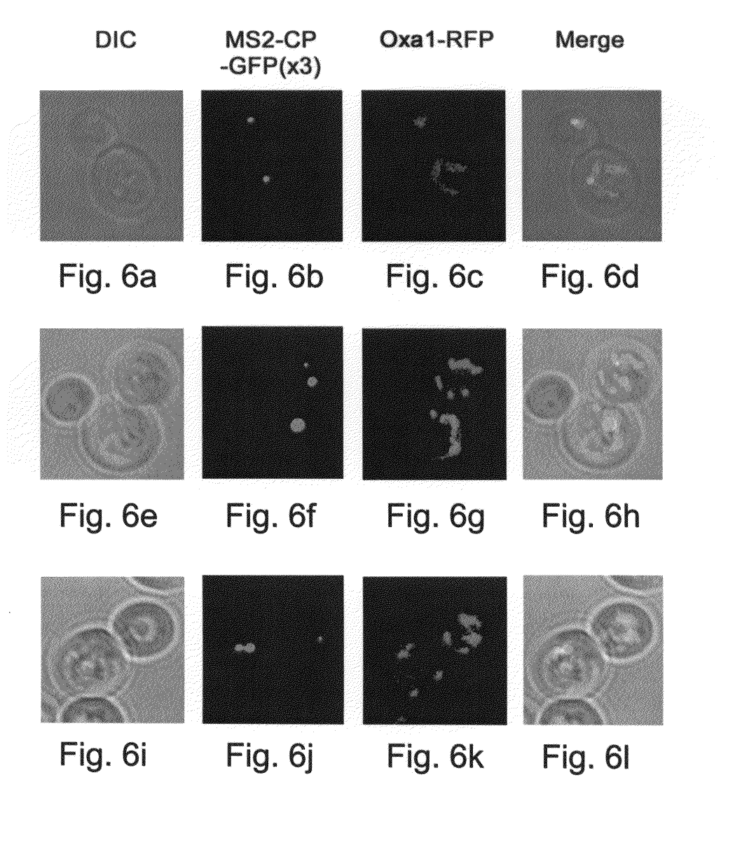

OXA1 mRNA localizes to the mitochondria in live yeast cells—Finally, the localization of OXA1, a mitochondria-localized mRNA in yeast (22), was demonstrated using the in vivo localization method of the present invention. A strain including the OXA1::loxP::MS2L::OXA1^{3′-UTR} cassette was created and examined for the localization of OXA1 mRNA in cells expressing MS2-GFP(x3). As is shown in FIGS. 6a-l, fluorescent OXA1 mRNA granules were non-polarized (only 14% of small- and medium-budded cells had OXA1 mRNA at the bud tip; n=50 cells) and co-localized with Oxa1-mRFP protein in 82% of cells (n=50). The tubular and punctate mitochondrial morphology observed with Oxa1-mRFP is typical of yeast mitochondria and the granular labeling of OXA1 mRNA on mitochondria using MS2-CP-GFP has been previously demonstrated using plasmid-based mRNA expression systems (22). Thus, the genomic tagging methodology of the present invention allows for the detection of organelle-associated mRNAs. It should be noted that the quantity of fluorescent OXA1 mRNA granules observed was not as abundant as that seen upon expression of a reporter mRNA bearing the OXA1 3′-UTR and MS2 loops using a plasmid-based system (22). This reduction is probably due to the lower levels of endogenous mRNA expression.

Altogether, these results suggest that the tagging of individual genes with RBP binding sites is efficient and leads to the detection of granular mRNA upon MS2-GFP(x2) or MS2-GFP(x3) expression. Thus, the mRNA-tagging approach (m-TAG) of the present invention can be employed to map the localization of all endogenous mRNAs in yeast—the mRNA locome—in a simple and rapid fashion.

### Example 3

### Visualization of Endogenous mRNA and Protein Localization In Vivo

In addition to detection of endogenous mRNA localization in vivo, the inventors have also incorporated the monomeric red fluorescent protein (mRFP) gene upstream to the first loxP site in the integration construct (for schematic representation of the construct see FIG. 1b). This inclusion ablates the stop codon of the gene-of-interest and places MRFP in-frame to the coding sequence at the 3′ end of the gene. By using this integration construct co-detection in vivo of both endogenous mRNA and protein localization can be performed. Importantly, this construct allows for the proper determination of protein localization using a system which does not remove the endogenous 3′-UTR sequence, unlike that previously used to integrate GFP at the 3′ end of genes (11).

The integration construct has the mRFP gene located upstream to the first loxP site, which itself is 5′ to the SpHIS5 selection marker and MS2 loop sequences. The integration construct can be easily adapted to any gene-of-interest by PCR amplification using specific primers. The forward primer includes nucleotide sequence from the 3′-end of the gene-of-interest, wherein the stop codon is altered, fused to a sequence derived from the 5′ end of mRFP lacking its start codon. This ensures that the translated protein is a full-length fusion with mRFP. The reverse primer includes sequence from the 5′-end of the 3′-UTR of the gene of interest fused to a sequence that corresponds to the plasmid sequence downstream (3′) of the MS2-CP binding sites (MS2L). After integration, Cre-mediated excision of the SpHIS5 selection marker allows for transcription of an mRNA that includes MS2-CP binding sites and the 3′-UTR, and enables translation of the gene-of-interest fused with mRFP. Upon expression, MS2L tagged mRNAs can bind to MS2-CP-GFP(X3), which is co-expressed in the cells, to visualize the mRNA (by GFP fluorescence).

Correspondingly, red fluorescence indicates protein localization. To visualize ASH1 mRNA and Ash1-mRFP protein, an ASH1::mRFP::loxP::MS2L::ASH1^{3′-UTR} has been constructed and examined.

### Example 4

### Visualization of Endogenous Protein Localization In Vivo

The inventors have also incorporated the mRFP gene upstream to the first loxP site, without MS2L sequences in the integration construct (for schematic representation of the construct see FIG. 1c). This inclusion ablates the stop codon of the gene-of-interest and places mRFP in-frame to the coding sequence at the 3′ end of the gene. By using this integration construct detection in vivo of endogenous protein localization can be performed. Importantly, this construct allows for the proper determination of protein localization using a system which does not remove the endogenous 3′-UTR sequence, unlike that previously used to integrate GFP at the 3′ end of genes (11).

The integration construct has the mRFP gene located upstream to the first loxP site, which itself is 5′ to the SpHIS5 selection marker. The integration construct can be easily adapted to any gene-of-interest by PCR amplification using specific primers. The forward primer includes nucleotide sequence from the 3′-end of the gene-of-interest, wherein the stop codon is altered, fused to a sequence derived from the 5′ end of mRFP lacking its start codon. This ensures that the translated protein will be a full-length fusion with mRFP. The reverse primer includes sequence from the 5′-end of the 3′-UTR of the gene of interest fused to a sequence that corresponds to the plasmid sequence downstream (3′) of the second loxP site. After integration, Cre-mediated excision of the SpHIS5 selection marker allows for transcription of an mRNA that includes the 3′-UTR, and enables translation of the gene-of-interest fused with mRFP. Correspondingly, red fluorescence indicates protein localization. To visualize Ash1-mRFP protein, an ASH1: :mRFP: :loxP: :ASH1^{3′-UTR} strain was constructed and examined.

### Example 5

### Localization of mRNAS Encoding Peroxisomal Proteins: Peroxins

Peroxins are proteins that participate in peroxisome biogenesis, which includes membrane formation, protein import into the peroxisomal matrix, and proliferation of the organelle. Genetic and biochemical methods have been used to identify the 25 peroxins (PEX) in yeast. Many peroxins are membrane proteins that have no known peroxisome targeting sequence (PTS). The mechanism by which these proteins localize to the peroxisome is not totally clear. One way to achieve peroxisomal localization might be through mRNA localization and translocation upon translation. While PEX3 mRNA was shown earlier to be localized to the endoplasmic reticulum (ER) (Aronov et al., 2007), other peroxin proteins were found to be localized to the vicinity of peroxisome. To examine the localization of endogenous mRNAs encoding the Peroxin proteins, the present inventors have used the mRNA localization method described hereinabove, as follows.

Experimental Results

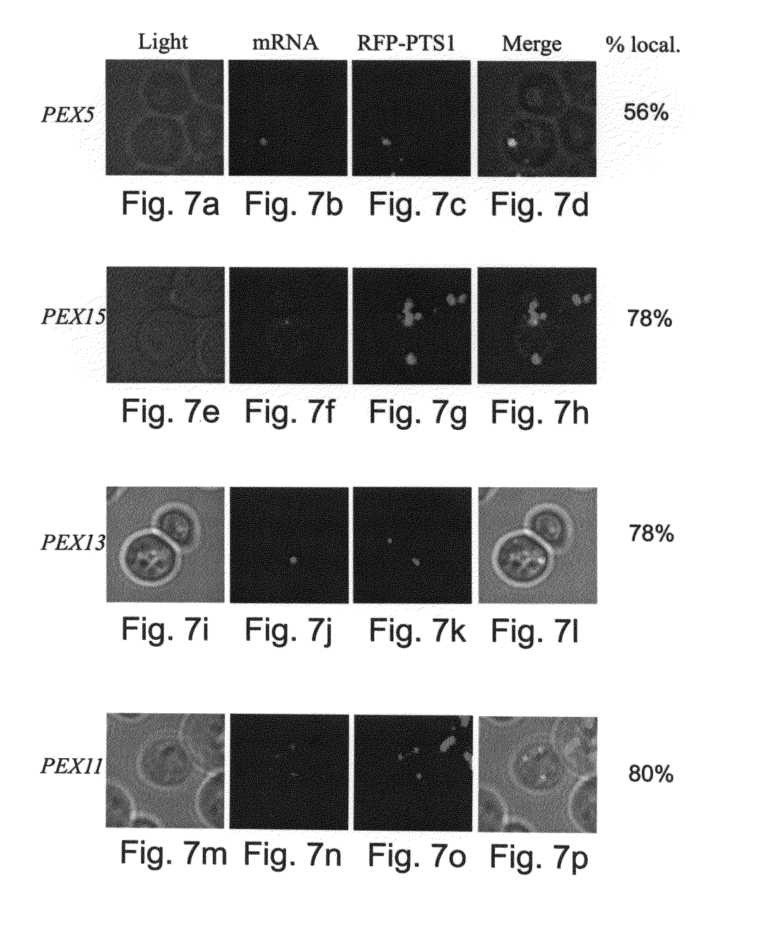

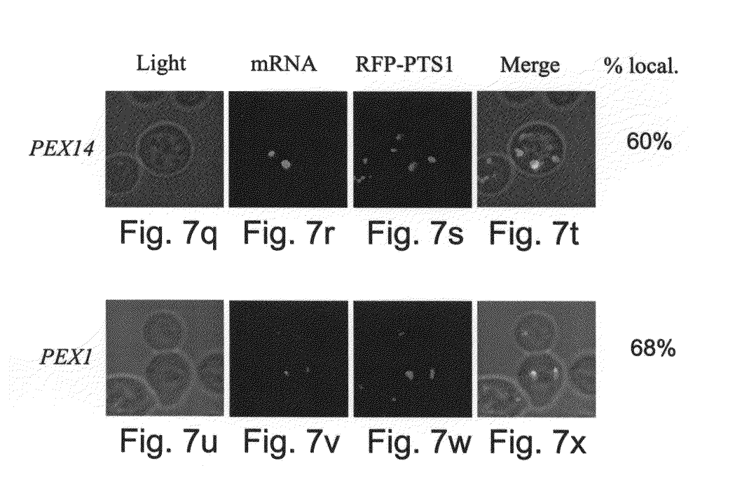

PEX14-Pex14 (Peroxin 14) (GenBank Accession No. and primers are provided in Table 1, hereinabove) is a peroxisomal membrane protein that is a central component of the peroxisomal protein import machinery. Pex14p (Peroxin 14 protein) interacts with the peroxisome targeting sequence 1 (PTS1) of Pex5 (Peroxin 5) and peroxisome targeting sequence 2 (PTS2) in Pex7 (Peroxin 7). To examine the localization of endogenous PEX14 mRNA, the m-TAG method of the invention was employed. However, no GFP granules were observed when integrated cells were grown on glucose-containing medium (Data not shown). As many peroxisomal genes are induced when yeast cells are grown on fatty acid-containing medium, PEX14_{INT} cells were grown on oleate-containing synthetic medium (SC, 0.2% Glucose, 0.2% Oleate, 0.25% Tween). Co-localization between endogenous PEX14 mRNA and peroxisomal marker (RFP-PTS1) was observed in 60% of cells expressing both RFP and GFP (n=50) on oleate-containing medium (FIGS. 7q-t). As a control, ASH1 integrated cells were transformed with RFP-PTS1. However, only a low correlation was seen between ASH1 mRNA and the peroxisomal marker (10% co-localization, n=30) (Data not shown).

PEX13-Pex13 (Peroxin 13) (GenBank Accession No. and primers are provided in Table 1, hereinabove) is an integral peroxisomal membrane receptor for the PTS1 peroxisomal matrix protein signal recognition factor Pex5. Pex13p has a src homology 3 (SH3) domain and interacts with Pex4. m-TAG was used to examine the endogenous localization of PEX13 mRNA and the co-localization between endogenous PEX13 mRNA and a peroxisomal marker (RFP-PTS1) was observed in 78% of the cells (grown on oleate) expressing both RFP and GFP (n=50) (FIGS. 7i-l).

PEX11- Pex11 (Peroxin 11) (GenBank Accession No. and primers are provided in Table 1, hereinabove) is a peroxisomal inner membrane protein required for peroxisome proliferation and medium-chain fatty acid oxidation. As the PEX11 promoter contains an oleate responsive element (ORE), the PEX11 integrated cells were also induced by oleate. Co-localization of PEX11 integrated cells with RFP-PTS1 was observed in 80% of the cells (n=50) (FIGS. 7m-p).

PEX15-Pex15p (Peroxin 15) (GenBank Accession No. and primers are provided in Table 1, hereinabove) is a tail-anchored type II (N_{cyt}-Cₗᵤₘₑₙ) integral peroxisomal membrane protein. Pex15p has a crucial role in peroxisomal matrix protein import and cells lacking Pex15 are characterized by the mislocalization of those proteins. O-glycosylation of Pex15 was observed when overproduced indicating that its carboxy-terminal tail might protrude into the ER. Thus, Pex15 may be targeted to peroxisomes via the ER, or to both peroxisomes and the ER. Co-localization between endogenous PEX15 mRNA and peroxisomal marker (RFP-PTS1) was observed in 78% of cells expressing both RFP and GFP (n=50) on oleate containing medium (no granules were seen on YPD medium) (FIGS. 7e-h). These results suggest that PEX15 mRNA localizes to peroxisomes.

PEX1-Pex 1 (Peroxin 1) (GenBank Accession No. and primers are provided in Table 1, hereinabove) is an AAA-family ATPase peroxin that has a crucial role in peroxisome biogenesis. PEX1 mutations are responsible for 50% of the Zellweger Syndrome cases, an autosomal-recessive disease that characterized by reduction or absence of peroxisomes. Co-localization between endogenous PEX1 mRNA and peroxisomal marker (RFP-PTS1) was observed in 68% of cells expressing both RFP and GFP (n=50) on oleate-containing medium (FIGS. 7u-x).

PEX5-Pex5 (Peroxin 5) (GenBank Accession No. and primers are provided in Table 1, hereinabove) functions as receptor for the C-terminal tripeptide signal sequence (PTS1) of peroxisomal matrix proteins, and is required for peroxisomal matrix protein import. Co-localization between endogenous PEX5 mRNA and peroxisomal marker (RFP-PTS1) was observed in 56% of cells expressing both RFP and GFP (n=50) on oleate-containing medium (FIGS. 7a-d). Interestingly, Pex5 can be found in the cytosol as well as the vicinity of peroxisomes (reviewed in Stanley, W. A. and Wilmanns, S. (2006) Dynamic architecture of the peroxisomal import receptor Pex5p. Biochem. Biophys. Acta 1763:1592-8). Induction with oleate may result in accumulation of PEX5 mRNA and protein to the vicinity of peroxisomes.

### Example 6

### Peroxisomal Matrix Proteins

Peroxisomal matrix proteins participate in variety of processes which include β-oxidation, synthesis of bile acids and cholesterol, detoxification of hydrogen peroxide (H₂O₂), and more. Most of the proteins have a peroxisomal targeting sequence (PTS) which is recognized by cytosolic receptor. In some cases, however, there is no known PTS and the targeting mechanism is still unrevealed. Moreover, mRNA localization might function as an additional mechanism to a protein targeting sequence as found in mitochondria. The present inventors have identified the mRNA localization of the peroxisomal matrix proteins using the m-TAG method, as follows.

Experimental Results

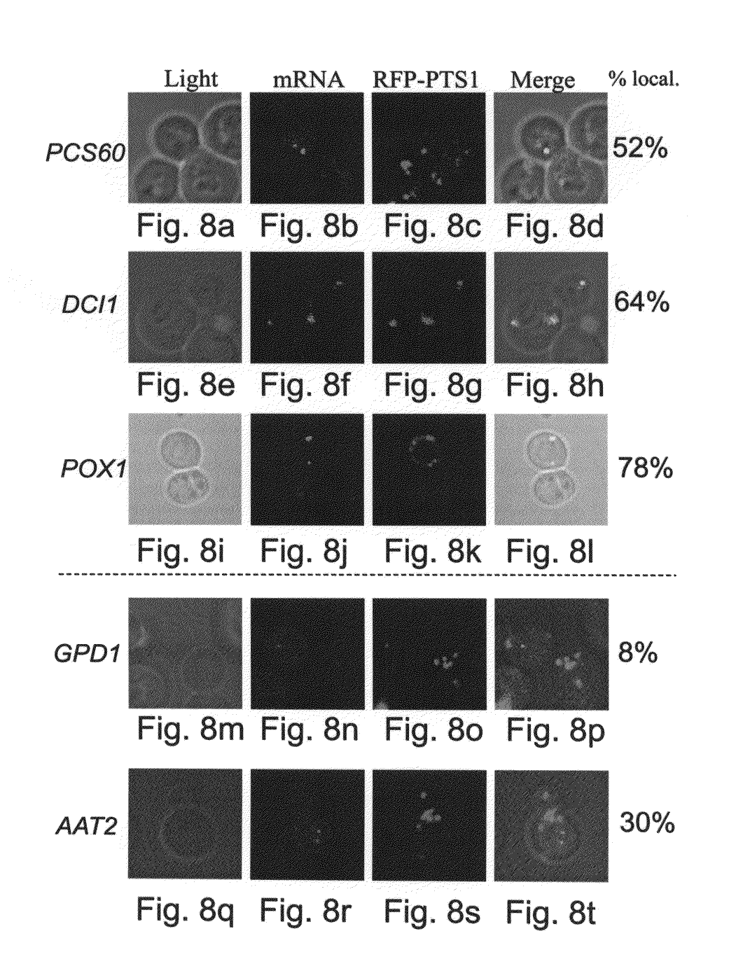

AAT2-Aat2 is an aspartate aminotransferase (GenBank Accession No. and primers are provided in Table 1, hereinabove) that is involved in nitrogen metabolism. It catalyzes the reversible transfer of the amino group from L-aspartate to 2-oxoglutarate to form oxaloacetate and L-glutamate. Co-localization between endogenous AAT2 mRNA and peroxisomal marker (RFP-PTS1) was observed in 30% of the cells grown in YPD medium (FIGS. 8q, r, s, t) and 32% in oleate medium (data not shown) (n=50). Interestingly, Aat2 is usually cytosolic and localized to peroxisomes when grown in oleate. These results suggest that, as expected, translation occurs in cytoplasm and the protein is post-translationally targeted to peroxisomes when induced by oleate.

GPD1-Gpd1 is a NAD-dependent glycerol-3-phosphate dehydrogenase (GenBank Accession No. and primers are provided in Table 1, hereinabove) essential for growth under osmotic stress. Co-localization between endogenous GPD1 mRNA and peroxisomal marker (RFP-PTS1) was observed in only 8% of the cells grown in YPD medium (FIGS. 8m, n, o, p). Gpd1 is known to be localized to cytosol, in addition to peroxisomes, which might explain why not many mRNA granules localized to the vicinity of peroxisomes.

DCI1-Dci1 is a peroxisomal delta(3,5)-delta(2,4)-dienoyl-CoA isomerase (GenBank Accession No. and primers are provided in Table 1, hereinabove) which is involved in β-oxidation of fatty acid. As shown in FIGS. 8e, f, g, h, the present inventors have found co-localization between DCI1 mRNA and peroxisomes (marked by RFP-PTS1) in 64% of the cells which express both GFP and RFP (n=50). Though having putative PTS1 and PTS2 sequences, Karpichev IV and Small GM (J Cell Sci. 2000, 113: 533-44) have shown that Dci1 localizes to peroxisomes when those sequences are deleted or even in the absence of the PTS receptors. The results shown here suggest that there is an additional mechanism for the localization of Dci1 to the peroxisome via mRNA localization.

POX1-Pox1 is a fatty-acyl coenzyme A oxidase (GenBank Accession No. and primers are provided in Table 1, hereinabove) involved in the fatty acid beta-oxidation pathway and is localized to the matrix of the peroxisomal matrix. Although having neither the PTS1 nor PTS2 consensus sequences, Pox1 has been shown to localize to peroxisomes. A possible mechanism for this localization could be through mRNA localization. In order to examine the endogenous localization of POX1 mRNA the PGI method was applied. Oleic acid was used to up-regulate the different peroxisomal enzymes, such as Pox1. Co-localization between endogenous POX1 mRNA and peroxisomal marker (RFP-PTS1) was observed in 78% of the cells expressing both RFP-PTS1 and MS2-CP-GFP(x3) (n=50) (FIGS. 8i-l).

PCS60-Pcs60 is a peroxisomal AMP-binding protein (GenBank Accession No. and primers are provided in Table 1, hereinabove), which localizes to both the peroxisomal membrane and the matrix. It has a PTS1 sequence at the C-terminus. Co-localization between endogenous PCS60 mRNA and peroxisomal marker (RFP-PTS1) was observed in 52% of the cells expressing both RFP-PTS1 and MS2-CP-GFP(x3) (n=50) (FIGS. 8a-d).

### Example 7

### Completion of the mRNA Localization Map for Peroxisomal Proteins

The integration of MS2 loops into the other 30⁺ genes encoding peroxisomal proteins is ongoing (Table 5, herein below). The combination of m-TAG and cellular fractionation studies helps to achieve a more complete picture of the localization of mRNAs encoding peroxisomal proteins. Discovering mRNA molecules that localize to peroxisomes, while others that do not, is an important step towards revealing the mechanisms by which mRNA molecules localize to peroxisomes and peroxisomal proteins reach their target. This work demonstrates, for the first time, the localization of mRNAs encoding peroxisomal proteins to the vicinity of the peroxisomes.

TABLE 6
Localization of endogenous mRNAs encoding peroxisomal proteins

| | | | Localization | | |
|---|---|---|---|---|---|
| | MS2L | Visual- | | Perox- | |
| Gene | tagging | ization | ER | isome | Other |
| AAT2 | ✓ | ✓ | | ✓ (30%) | ✓ (70%) |
| NC_001144 | | | | | |
| (196830-198086) | | | | | |
| ANT1 | ✓ | | | | |
| NC_001139 | | | | | |
| (469097-472303) | | | | | |
| CAT2 | | | | | |
| NC_001145 | | | | | |
| (192788-194800) | | | | | |
| CIT2 | | | | | |
| NC_001135 | | | | | |
| (120944-122326) | | | | | |
| CTA1 | ✓ | | | | |
| NC_001136 | | | | | |
| (968129-969676) | | | | | |
| DCI1 | ✓ | ✓ | | ✓ (64%) | |
| NC_001147 | | | | | |
| (675168-674353) | | | | | |
| ECI1 | ✓ | | | | |
| NC_001144 | | | | | |
| (706200-707042) | | | | | |
| FAA2 | ✓ | | | | |
| NC_001137 | | | | | |
| (184540-186774) | | | | | |
| FAT1 | ✓ | | | | |
| NC_001134 | | | | | |
| (318266-320275) | | | | | |
| FOX2 | ✓ | | | | |
| NC_001143 | | | | | |
| (456697-453995) | | | | | |
| GPD1 | ✓ | | | | |
| NC_001136 | | | | | |
| (411822-412997) | | | | | |
| INP1 | ✓ | | | | |
| NC_001145 | | | | | |
| (670062-671324) | | | | | |
| INP2 | ✓ | | | | |
| NC_001145 | | | | | |
| (584270-586387) | | | | | |
| MDH3 | ✓ | ✓ | | ✓ (42%) | |
| NC_001136 | | | | | |
| (315357-316388) | | | | | |
| NPY1 | ✓ | | | | |
| NC_001139 | | | | | |
| (376104-377258) | | | | | |
| PCD1 | ✓ | ✓ | | ✓ (8%) | ✓ (92%) |
| NC_001144 | | | | | |
| (441716-442738) | | | | | |
| PCS60 | ✓ | ✓ | ✓ | ✓ (52%) | |
| NC_001134 | | | | | |
| (668346-666715) | | | | | |
| PEX1 | ✓ | ✓ | | ✓ (68%) | |
| NC_001143 | | | | | |
| (73870-70739) | | | | | |
| PEX2 | ✓ | | | | |
| NC_001142 | | | | | |
| (36919-37734) | | | | | |
| PEX3 | ✓ | ✓ | ✓ (80%) | | |
| NC_001136 | | | | | |
| (1127590- | | | | | |
| 1126265) | | | | | |
| PEX4 | | | | | |
| NC_001139 | | | | | |
| (756901-757452) | | | | | |
| PEX5 | ✓ | ✓ | | ✓ (56%) | |
| NC_001136 | | | | | |
| (950559-952397) | | | | | |
| PEX6 | ✓ | | | | |
| NC_001146 | | | | | |
| (19541-22633) | | | | | |
| PEX7 | ✓ | | | | |
| NC_001136 | | | | | |
| (740470-741597) | | | | | |
| PEX8 | ✓ | | | | |
| NC_001139 | | | | | |
| (637748-639517) | | | | | |
| PEX10 | ✓ | | | | |
| NC_001136 | | | | | |
| (998860-999873) | | | | | |
| PEX11 | ✓ | ✓ | | ✓ (80%) | |
| NC_001147 | | | | | |
| (47932-48642) | | | | | |
| PEX12 | ✓ | ✓ | | ✓ (58%) | |
| NC_001145 | | | | | |
| (324235-325434) | | | | | |
| PEX13 | ✓ | ✓ | | ✓ (78%) | |
| NC_001144 | | | | | |
| (537274-538434) | | | | | |
| PEX14 | ✓ | ✓ | | ✓ (60%) | |
| NC_001139 | | | | | |
| (216278-217303) | | | | | |
| PEX15 | ✓ | ✓ | | ✓ (78%) | |
| NC_001147 | | | | | |
| (247149-248300) | | | | | |
| PEX17 | ✓ | | | | |
| NC_001146 | | | | | |
| (245618-246217) | | | | | |
| PEX18 | ✓ | | | | |
| NC_001140 | | | | | |
| (420075-419224) | | | | | |
| PEX19 | ✓ | | | | |
| NC_001136 | | | | | |
| (337277-336249) | | | | | |
| PEX21 | ✓ | | | | |
| NC_001139 | | | | | |
| (970058-969192) | | | | | |
| PEX22 | ✓ | | | | |
| NC_001133 | | | | | |
| (42178-42720) | | | | | |
| PEX25 | ✓ | | | | |
| NC_001148 | | | | | |
| (337435-338619) | | | | | |
| PEX27 | ✓ | | | | |
| NC_001147 | | | | | |
| (710447-711577) | | | | | |
| PEX28 | ✓ | | | | |
| NC_001140 | | | | | |
| (397254-398993) | | | | | |
| PEX29 | ✓ | | | | |
| NC_001136 | | | | | |
| (1415202- | | | | | |
| 1416866) | | | | | |
| PEX30 | ✓ | | | | |
| NC_001144 | | | | | |
| (779215-780786) | | | | | |
| PEX31 | | | | | |
| NC_001139 | | | | | |
| (502942-504330) | | | | | |
| PEX32 | | | | | |
| NC_001134 | | | | | |
| (572366-573607) | | | | | |
| POT1 | ✓ | ✓ | | ✓ (24%) | ✓ (76%) |
| NC_001141 | | | | | |
| (41444-40191) | | | | | |
| POX1 | ✓ | ✓ | | ✓ (78%) | |
| NC_001139 | | | | | |
| (108162-110408) | | | | | |
| PXA1 | | | | | |
| NC_001148 | | | | | |
| (273254-275866) | | | | | |
| PXA2 | | | | | |
| NC_001143 | | | | | |
| (86230-88791) | | | | | |
| SPS19 | ✓ | | | | |
| NC_001146 | | | | | |
| (259579-260457) | | | | | |
| TES1 | ✓ | | | | |
| NC_001142 | | | | | |
| (468196-467147) | | | | | |
| YOR084W | | | | | |
| NC_001147 | | | | | |
| (480589-481752) | | | | | |

Table 6: Tagging and visualization of endogenous mRNAs encoding peroxisomal proteins in wild-type cells in vivo. Where indicated (by a ✓) endogenous cells were tagged with MS2L (MS2 loops), grown on oleate-containing medium to induce peroxisomes, and (where indicated) mRNA localization was visualized by fluorescence microscopy. Localization of granular mRNA to the ER or peroxisome is indicated in percent. At least 50 cells were scored for each tagged gene.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

1. Beach, D. L., Salmon, E. D. & Bloom, K. Localization and anchoring of mRNA in budding yeast. Curr Biol 9, 569-578 (1999).
2. Bertrand, E. et al. Localization of ASH1 mRNA particles in living yeast. Mol Cell 2, 437-445 (1998).
3. Bashirullah, A., Cooperstock, R. L. & Lipshitz, H. D. RNA localization in development. Annu Rev Biochem 67, 335-394 (1998).
4. Condeelis, J. & Singer, R. H. How and why does beta-actin mRNA target? Biol

Cell 97, 97-110 (2005).
5. Gonsalvez, G. B., Urbinati, C. R. & Long, R. M. RNA localization in yeast: moving towards a mechanism. Biol Cell 97, 75-86 (2005).
6. Kloc, M., Zearfoss, N. R. & Etkin, L. D. Mechanisms of subcellular mRNA localization. Cell 108, 533-544 (2002).
7. Sotelo-Silveira, J. R., Calliari, A., Kun, A., Koenig, E. & Sotelo, J. R. RNA trafficking in axons. Traffic 7, 508-515 (2006).
8. Wodarz, A. Establishing cell polarity in development. Nat Cell Biol 4, E39-44 (2002).
9. Giaever, G. et al. Functional profiling of the Saccharomyces cerevisiae genome. Nature 418, 387-391 (2002).
10. Ghaemmaghami, S. et al. Global analysis of protein expression in yeast. Nature 425, 737-741 (2003).
11. Huh, W. K. et al. Global analysis of protein localization in budding yeast. Nature 425, 686-691 (2003).
12. Fouts, D. E., True, H. L. & Celander, D. W. Functional recognition of fragmented operator sites by R17/MS2 coat protein, a translational repressor. Nucleic Acids Res 25, 4464-4473 (1997).
13. Sauer, B. Functional expression of the cre-lox site-specific recombination system in the yeast Saccharomyces cerevisiae. Mol Cell Biol 7, 2087-2096 (1987).
14. Aronov, S., Gelin-Licht, R., Zipor, G., Haim, L., Safran, E. & Gerst, J. E. mRNAs encoding polarity and exocytosis factors are co-transported with cortical endoplasmic reticulum to the incipient bud in yeast. Mol Cell Biol 27, 3441-3455 (2007).
15. Guldener, U., Heck, S., Fielder, T., Beinhauer, J. & Hegemann, J. H. A new efficient gene disruption cassette for repeated use in budding yeast. Nucleic Acids Res 24, 2519-2524 (1996).
16. Long, R. M. et al. Mating type switching in yeast controlled by asymmetric localization of ASH1 mRNA. Science 277, 383-387 (1997).
17. Takizawa, P. A., Sil, A., Swedlow, J. R., Herskowitz, I. & Vale, R. D. Actin-dependent localization of an RNA encoding a cell-fate determinant in yeast. Nature 389, 90-93 (1997).
18. Jansen, R. P., Dowzer, C., Michaelis, C., Galova, M. & Nasmyth, K. Mother cell-specific HO expression in budding yeast depends on the unconventional myosin myo4p and other cytoplasmic proteins. Cell 84, 687-697 (1996).
19. Aronov, S. & Gerst, J. E. Involvement of the late secretory pathway in actin regulation and mRNA transport in yeast. J Biol Chem 279, 36962-36971 (2004).
20. Grosshans, B. L. et al. The yeast Ig1 family member Sro7p is an effector of the secretory Rab GTPase Sec4p. J Cell Biol 172, 55-66 (2006).
21. Hoepfner, D., Schildknegt, D., Braakman, I., Philippsen, P. & Tabak, H. F. Contribution of the endoplasmic reticulum to peroxisome formation. Cell 122, 85-95 (2005).
22. Sylvestre, J., Margeot, A., Jacq, C., Dujardin, G. & Corral-Debrinski, M. The role of the 3′ untranslated region in mRNA sorting to the vicinity of mitochondria is conserved from yeast to human cells. Mol Biol Cell 14, 3848-3856 (2003).

1. An isolated polynucleotide comprising a first nucleic acid sequence which comprises two functionally compatible recognition sites for a site-specific recombination enzyme and a second nucleic acid sequence encoding a protein binding-RNA sequence.

2. The isolated polynucleotide of claim 1, further comprising a third nucleic acid sequence encoding a reporter polypeptide.

3-4. (canceled)

5. A nucleic acid construct comprising the isolated polynucleotide of claim 1.

6. A cell transformed with the nucleic acid construct of claim 5.

7. A system comprising:
the isolated polynucleotide of claim 1;
(ii) a second isolated polynucleotide comprising a first nucleic acid sequence which comprises two functionally compatible recognition sites for a site-specific recombination enzyme and a second nucleic acid sequence encoding a reporter polypeptide.

8. (canceled)

9. A transformed cell having a genome which comprises an exogenous polynucleotide being transcriptionally regulated by endogenous 5′ and 3′-untranslated regions of a gene-of-interest, said exogenous polynucleotide comprising a first nucleic acid sequence which comprises at least one recognition site for a site-specific recombination enzyme, and a second nucleic acid sequence encoding a reporter polypeptide.

10. The isolated polynucleotide of claim 1, further comprising additional nucleic acid sequences which enable homologous recombination with a gene-of-interest.

11. A method of identifying a localization of an RNA encoded by a gene-of-interest within a cell, the method comprising:
(a) introducing into the cell the isolated polynucleotide of claim 10 so as to enable homologous recombination of said isolated polynucleotide between endogenous 5′ and 3′-untranslated regions of the gene-of-interest; and
(b) detecting the RNA encoded by the gene-of-interest via said protein binding-RNA sequence;
thereby identifying the localization of the RNA encoded by the gene-of-interest within the cell.

12. A kit for identifying a localization of an RNA encoded by a gene-of-interest within a cell, the kit comprising:
(i) the isolated polynucleotide of claim 1; and
(ii) a pair of oligonucleotides which enable homologous recombination of the isolated polynucleotide between endogenous 5′ and 3′-untranslated regions of the gene-of-interest.

13. The kit of claim 12, wherein said pair of oligonucleotides is selected from the group of oligonucleotide pairs consisting of SEQ ID NOs:1 and 2, 3 and 4, 5 and 6, 7 and 8, 9 and 10, 11 and 12, 13 and 14, 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 106 and 107, 108 and 109, 110 and 111, 112 and 113, 114 and 115, 116 and 117, 118 and 119, 120 and 121, 122 and 123, 124 and 125, 126 and 127, 128 and 129, 130 and 131, 132 and 133, 134 and 135, 136 and 137, 138 and 139, 140 and 141, 142 and 143, 144 and 145, 146 and 147, 148 and 149, 150 and 151, 152 and 153, 154 and 155, 156 and 157, 158 and 159, 160 and 161, 162 and 163, 164 and 165, 166 and 167, 168 and 169 and 170 and 171.

14. (canceled)

15. A method of identifying a localization of a polypeptide encoded by a gene-of-interest within a cell, the method comprising:
(a) introducing into the cell an isolated polynucleotide capable of homologous recombination between endogenous 5′ and 3′-untranslated regions of the gene-of-interest, said isolated polynucleotide comprising a first nucleic acid sequence which comprises two functionally compatible recognition sites for a site-specific recombination enzyme, and a second nucleic acid sequence encoding a reporter polypeptide; and
(b) detecting within the cell a presence of said reporter polypeptide;
thereby identifying the localization of the polypeptide encoded by the gene-of-interest within the cell.

16-17. (canceled)

18. A method of identifying a localization of an RNA and/or a polypeptide encoded by a gene-of-interest within a cell, the method comprising:
(a) introducing into the cell the isolated polynucleotide of claim 10 so as to enable homologous recombination of said isolated polynucleotide between endogenous 5′ and 3′-untranslated regions of the gene-of-interest;
(b) detecting the RNA encoded by the gene-of-interest via said protein binding-RNA sequence; and/or
(c) detecting said reporter polypeptide;
thereby identifying the localization of the RNA and/or the polypeptide encoded by the gene-of-interest within the cell.

19. The method of claim 11, wherein said detecting said RNA encoded by the gene-of-interest is effected by expressing within the cell an exogenous polynucleotide encoding a polypeptide capable of binding said protein binding-RNA sequence.

20. The method of claim 11, wherein said detecting said RNA encoded by the gene-of-interest is effected by introducing into the cell an exogenous polypeptide capable of binding said protein binding-RNA sequence.

21. (canceled)

22. A kit for identifying a localization of an RNA and/or a polypeptide encoded by a gene-of-interest within a cell, the kit comprising:
(i) the isolated polynucleotide of claim 2; and
(ii) a pair of oligonucleotides which enable homologous recombination of said isolated polynucleotide between endogenous 5′ and 3′-untranslated regions of the gene-of-interest.

23. The kit of claim 22, wherein said pair of oligonucleotides is selected from the group of oligonucleotide pairs consisting of SEQ ID NOs:91 and 2, 93 and 4, 95 and 6, and 97 and 8.

24-27. (canceled)

28. The isolated polynucleotide, of claim 1, wherein said first nucleic acid sequence further comprising a selectable marker.

29. The isolated polynucleotide, of claim 28, wherein said two functionally compatible recognition sites are positioned so as to enable excision of said selectable marker following homologous recombination of said isolated polynucleotide in a genome of a cell.

30. The isolated polynucleotide, of claim 1, wherein each of said two functionally compatible recognition sites for said site-specific recombination enzyme comprises a loxP sequence.

31. The isolated polynucleotide, of claim 1, wherein said site-specific recombination enzyme comprises a Cre recombinase.

32. The isolated polynucleotide, of claim 1, wherein said protein binding-RNA sequence is capable of binding a protein selected from the group consisting of a bacteriophage MS2 coat protein, an IRP1 protein, a zipcode binding protein, a box C/D snoRNA binding protein and an aptamer.

33. The method of claim 11, wherein the cell is a living cell.

34. The transformed cell, of claim 9, wherein the cell is a eukaryotic cell.

35. The transformed cell, of claim 9, wherein the cell is a yeast cell.

36. The isolated polynucleotide, of claim 2, wherein said reporter polypeptide comprises an antibody binding antigen or a labeled protein.

37. The method of claim 11, wherein the RNA encoded by the gene-of-interest is selected from the group consisting of ASH1, SRO7, PEX3, OXA1, PEX14, PEX13, PEX11, PEX15, PEX1, PEX5, AAT2, GPD1, DCI1, POX1, PCS60, MDH3, PCD1, PEX12 and POT1.

38. The method of claim 11, wherein the gene-of-interest is selected from the group consisting of a peroxin and a peroxisomal matrix protein.

39. A nucleic acid construct comprising the isolated polynucleotide of claim 2.

40. A cell transformed with the nucleic acid construct of claim 39.

41. The isolated polynucleotide of claim 2, further comprising additional nucleic acid sequences which enable homologous recombination with a gene-of-interest.


SEQUENCE LISTING (203 sequences)

SEQ ID NO: 1 - DNA, 60 bp, Artificial sequence
  Single strand DNA oligonucleotide
cttattttgtaattacataactgagacagtagagaattgaacgctgcaggtcgacaaccc

SEQ ID NO: 2 - DNA, 60 bp, Artificial sequence
  Single strand DNA oligonucleotide
atgtctcttattagttgaaagagattcagttatccatgtagcataggccactagtggatc

SEQ ID NO: 3 - DNA, 65 bp, Artificial sequence
  Single strand DNA oligonucleotide
gagcagactggaaaagatgtaatgaaaggtgcccttggtttttaaacgctgcaggtcgacaaccc

SEQ ID NO: 4 - DNA, 65 bp, Artificial sequence
  Single strand DNA oligonucleotide
atagaaggaagttgctcattaccctgtatgaattagtgtatgtatctgatatcgatcgcgcgcag

SEQ ID NO: 5 - DNA, 60 bp, Artificial sequence
  Single strand DNA oligonucleotide
aattgttcacaaatcaaacttcattaataacaaaaaatgaacgctgcaggtcgacaaccc

SEQ ID NO: 6 - DNA, 60 bp, Artificial sequence
  Single strand DNA oligonucleotide
tttatatttttatatttacagagagatatagagcctttatgcataggccactagtggatc

SEQ ID NO: 7 - DNA, 60 bp, Artificial sequence
  Single strand DNA oligonucleotide
caactttggcgtctccagctcgttttccttcaagccttaaacgctgcaggtcgacaaccc

SEQ ID NO: 8 - DNA, 60 bp, Artificial sequence
  Single strand DNA oligonucleotide
tatatatattctggtgtgagtgtcagtacttattcagagagcataggccactagtggatc

SEQ ID NO: 9 - DNA, 60 bp, Artificial sequence
  Single strand DNA oligonucleotide
tgtaatcatcgtccccattgctgttcactttagtcgatagacgctgcaggtcgacaaccc

SEQ ID NO: 10 - DNA, 60 bp, Artificial sequence
  Single strand DNA oligonucleotide
tatggaagctccctatatatatagcattgcgagtgaacttgcataggccactagtggatc

SEQ ID NO: 11 - DNA, 60 bp, Artificial sequence
  Single strand DNA oligonucleotide
taaacagcttcaagagggaaacaggcgccacaagttataaacgctgcaggtcgacaaccc

SEQ ID NO: 12 - DNA, 60 bp, Artificial sequence
  Single strand DNA oligonucleotide
gattttttatgttaaaatcctatctctaaatgctatattagcataggccactagtggatc

SEQ ID NO: 13 - DNA, 60 bp, Artificial sequence
  Single strand DNA oligonucleotide
cgccgctgtaaaactatcgcaggcaaaatctaaactataaacgctgcaggtcgacaaccc

SEQ ID NO: 14 - DNA, 60 bp, Artificial sequence
  Single strand DNA oligonucleotide
ataatcgctattttttatattattcaaatctttttttgtagcataggccactagtggatc

SEQ ID NO: 15 - DNA, 60 bp, Artificial sequence
  Single strand DNA oligonucleotide
aacttttgctaagagcagcagaaataagagtaagttgtagacgctgcaggtcgacaaccc

SEQ ID NO: 16 - DNA, 60 bp, Artificial sequence
  Single strand DNA oligonucleotide
tagaagctttcagagagcataaaattgtacaggatactgcgcataggccactagtggatc

SEQ ID NO: 17 - DNA, 60 bp, Artificial sequence
  Single strand DNA oligonucleotide
gaattccatcgacattggtagccgactctcccttatgtgaacgctgcaggtcgacaaccc

SEQ ID NO: 18 - DNA, 59 bp, Artificial sequence
  Single strand DNA oligonucleotide
tttaaagggaaacgcgctttgttcttttcttcttcctttgcataggccactagtggatc

SEQ ID NO: 19 - DNA, 60 bp, Artificial sequence
  Single strand DNA oligonucleotide
tgactggcaaaatggacaggtcgaagactccatcccatagacgctgcaggtcgacaaccc

SEQ ID NO: 20 - DNA, 60 bp, Artificial sequence
  Single strand DNA oligonucleotide
caatttccgttaaaaaactaattacttacatagaattgcggcataggccactagtggatc

SEQ ID NO: 21 - DNA, 60 bp, Artificial sequence
  Single strand DNA oligonucleotide
ccagattgtagggttgctaaaacttctagcgagtatatgaacgctgcaggtcgacaaccc

SEQ ID NO: 22 - DNA, 60 bp, Artificial sequence
  Single strand DNA oligonucleotide
aaataagtaggtagggttttataaactattcaaatatttcgcataggccactagtggatc

SEQ ID NO: 23 - DNA, 60 bp, Artificial sequence
  Single strand DNA oligonucleotide
tggtcttgagttccatgatgttgaagacagaattgcttaaacgctgcaggtcgacaaccc

SEQ ID NO: 24 - DNA, 60 bp, Artificial sequence
  Single strand DNA oligonucleotide
ctgaaattcatggtttaaattaaagaaatttcaaggcccggcataggccactagtggatc

SEQ ID NO: 25 - DNA, 60 bp, Artificial sequence
  Single strand DNA oligonucleotide
ccttgataaggaattaaccgacggttgcaaacaacaataaacgctgcaggtcgacaaccc

SEQ ID NO: 26 - DNA, 75 bp, Artificial sequence
  Single strand DNA oligonucleotide
gataatgaactacttttttttttttttttttactgttatcataaatatatataccgcataggccactagtggatc

SEQ ID NO: 27 - DNA, 60 bp, Artificial sequence
  Single strand DNA oligonucleotide
tttcgtcaaggacgaaattcacaaagacatacttgattgaacgctgcaggtcgacaaccc

SEQ ID NO: 28 - DNA, 60 bp, Artificial sequence
  Single strand DNA oligonucleotide
gtagtagttacaagaggtacaattgtagaaactgcctatagcataggccactagtggatc

SEQ ID NO: 29 - DNA, 60 bp, Artificial sequence
  Single strand DNA oligonucleotide
gatacatcgtgttattaagaatgcaacaccagtagcataaacgctgcaggtcgacaaccc

SEQ ID NO: 30 - DNA, 60 bp, Artificial sequence
  Single strand DNA oligonucleotide
aagaggggtggggttgtaggtgaaggaaaacaattgtgcagcataggccactagtggatc

SEQ ID NO: 31 - DNA, 60 bp, Artificial sequence
  Single strand DNA oligonucleotide
catggacctgaaaagatttaaaggagaattttcgttttgaacgctgcaggtcgacaaccc

SEQ ID NO: 32 - DNA, 60 bp, Artificial sequence
  Single strand DNA oligonucleotide
tgggcagtgatgcgagaacataaaattgcggagaaccatagcataggccactagtggatc

SEQ ID NO: 33 - DNA, 60 bp, Artificial sequence
  Single strand DNA oligonucleotide
tactggtatgggtgccgccgccatctttattaaagaatagacgctgcaggtcgacaaccc

SEQ ID NO: 34 - DNA, 60 bp, Artificial sequence
  Single strand DNA oligonucleotide
aataaaaagggagaatattaactattatcaagtattaaaagcataggccactagtggatc

SEQ ID NO: 35 - DNA, 60 bp, Artificial sequence
  Single strand DNA oligonucleotide
tgcagctaatgcggaaattttatcgaaaataaacaagtgaacgctgcaggtcgacaaccc

SEQ ID NO: 36 - DNA, 60 bp, Artificial sequence
  Single strand DNA oligonucleotide
cgcaaaacagagggttcgaaggaaaacaggaaacctctacgcataggccactagtggatc

SEQ ID NO: 37 - DNA, 60 bp, Artificial sequence
  Single strand DNA oligonucleotide
tagtgggagcggaaacagttctaaatcaaattgctgttgaacgctgcaggtcgacaaccc

SEQ ID NO: 38 - DNA, 60 bp, Artificial sequence
  Single strand DNA oligonucleotide
ttcacgattaattctcaaagaagcaaaaatcttcttttctgcataggccactagtggatc

SEQ ID NO: 39 - DNA, 60 bp, Artificial sequence
  Single strand DNA oligonucleotide
tccagaagccttaataaagagtatgacatctaaattataaacgctgcaggtcgacaaccc

SEQ ID NO: 40 - DNA, 60 bp, Artificial sequence
  Single strand DNA oligonucleotide
gaaagtgtcatataataatagtactcaatacctatacgtagcataggccactagtggatc

SEQ ID NO: 41 - DNA, 60 bp, Artificial sequence
  Single strand DNA oligonucleotide
tgtctacgggtcagaacgagacattcgagccaagttctgaacgctgcaggtcgacaaccc

SEQ ID NO: 42 - DNA, 60 bp, Artificial sequence
  Single strand DNA oligonucleotide
aatatatatgtatgtgtttatacgtgggagggaattgtccgcataggccactagtggatc

SEQ ID NO: 43 - DNA, 60 bp, Artificial sequence
  Single strand DNA oligonucleotide
gaatgaagctttggttaaaacgactaaacaaaaactgtaaacgctgcaggtcgacaaccc

SEQ ID NO: 44 - DNA, 60 bp, Artificial sequence
  Single strand DNA oligonucleotide
attattattgaataatatatgtaatagtgtacacaagtgtgcataggccactagtggatc

SEQ ID NO: 45 - DNA, 31 bp, Artificial sequence
  Single strand DNA oligonucleotide
cgtcgacctgaggtgatatcaacccgggccc

SEQ ID NO: 46 - DNA, 31 bp, Artificial sequence
  Single strand DNA oligonucleotide
gggcccgggttgatatcacctcaggtcgacg

SEQ ID NO: 47 - DNA, 30 bp, Artificial sequence
  Single strand DNA oligonucleotide
gatatcgtgtctaaaggtgaagaattattc

SEQ ID NO: 48 - DNA, 27 bp, Artificial sequence
  Single strand DNA oligonucleotide
gatatctttgtacaattcatccatacc

SEQ ID NO: 49 - DNA, 28 bp, Artificial sequence
  Single strand DNA oligonucleotide
gaattgtacaaagagatcaagcttatcg

SEQ ID NO: 50 - DNA, 28 bp, Artificial sequence
  Single strand DNA oligonucleotide
cgataagcttgatctctttgtacaattc

SEQ ID NO: 51 - DNA, 31 bp, Artificial sequence
  Single strand DNA oligonucleotide
tgactggtcgaccgatgttcaaactcacctc

SEQ ID NO: 52 - DNA, 31 bp, Artificial sequence
  Single strand DNA oligonucleotide
tgactgcccgggttttttgttattaatgaag

SEQ ID NO: 53 - DNA, 28 bp, Artificial sequence
  Single strand DNA oligonucleotide
agcgagctctaaaggctctatatctctc

SEQ ID NO: 54 - DNA, 31 bp, Artificial sequence
  Single strand DNA oligonucleotide
tgactggagctcatcgcaaggctgttttaag

SEQ ID NO: 55 - DNA, 29 bp, Artificial sequence
  Single strand DNA oligonucleotide
tcagtcacccggggcctcctccgaggacg

SEQ ID NO: 56 - DNA, 27 bp, Artificial sequence
  Single strand DNA oligonucleotide
tcagtcgagctcttaggcgccggtgga

SEQ ID NO: 57 - DNA, 45 bp, Artificial sequence
  Single strand DNA oligonucleotide
gacgatgaaagcttaggcgccggtgcctcctccgaggacgtcatc

SEQ ID NO: 58 - DNA, 27 bp, Artificial sequence
  Single strand DNA oligonucleotide
ctcagcttcctttcgggctttgttagc

SEQ ID NO: 59 - DNA, 21 bp, Artificial sequence
  Single strand DNA oligonucleotide
ctgcgaaattgaagggtaccg

SEQ ID NO: 60 - DNA, 21 bp, Artificial sequence
  Single strand DNA oligonucleotide
gcacagacaaggagagaaatg

SEQ ID NO: 61 - DNA, 21 bp, Artificial sequence
  Single strand DNA oligonucleotide
cgactatgctaccgccatggg

SEQ ID NO: 62 - DNA, 31 bp, Artificial sequence
  Single strand DNA oligonucleotide
caaaccttcgtaaaactagacatgtataatg

SEQ ID NO: 63 - DNA, 31 bp, Artificial sequence; identical to SEQ ID NO: 51
  Single strand DNA oligonucleotide

SEQ ID NO: 64 - DNA, 31 bp, Artificial sequence; identical to SEQ ID NO: 54
  Single strand DNA oligonucleotide

SEQ ID NO: 65 - DNA, 20 bp, Artificial sequence
  Single strand DNA oligonucleotide
gaacgaatacctggccactc

SEQ ID NO: 66 - DNA, 30 bp, Artificial sequence
  Single strand DNA oligonucleotide
tcagtcagtgagctcccgaacattgggcac

SEQ ID NO: 67 - DNA, 21 bp, Artificial sequence
  Single strand DNA oligonucleotide
gaaaagccatgtggtacaagg

SEQ ID NO: 68 - DNA, 25 bp, Artificial sequence
  Single strand DNA oligonucleotide
ataagaacaaagtaaatatacgccc

SEQ ID NO: 69 - DNA, 22 bp, Artificial sequence
  Single strand DNA oligonucleotide
cataagatggcctctcactagg

SEQ ID NO: 70 - DNA, 26 bp, Artificial sequence
  Single strand DNA oligonucleotide
ccgtatcagttttcaatataggatca

SEQ ID NO: 71 - DNA, 20 bp, Artificial sequence
  Single strand DNA oligonucleotide
ccatcccttgggttgtactg

SEQ ID NO: 72 - DNA, 20 bp, Artificial sequence
  Single strand DNA oligonucleotide
ttcaaatcagccctcaaagg

SEQ ID NO: 73 - DNA, 29 bp, Artificial sequence
  Single strand DNA oligonucleotide
atgacccgggatgagtgacgtggtcagta

SEQ ID NO: 74 - DNA, 26 bp, Artificial sequence
  Single strand DNA oligonucleotide
tcggagctcacaatatctagagcctc

SEQ ID NO: 75 - DNA, 30 bp, Artificial sequence
  Single strand DNA oligonucleotide
atgacccgggatgagtgcttccaaaatggc

SEQ ID NO: 76 - DNA, 19 bp, Artificial sequence
  Single strand DNA oligonucleotide
gatacccgctcgtgaaagg

SEQ ID NO: 77 - DNA, 30 bp, Artificial sequence
  Single strand DNA oligonucleotide
tccccgggatggatactatgaatacagcaa

SEQ ID NO: 78 - DNA, 28 bp, Artificial sequence
  Single strand DNA oligonucleotide
tggagctccttagttcaaacatatggtg

SEQ ID NO: 79 - DNA, 21 bp, Artificial sequence
  Single strand DNA oligonucleotide
ggcgatgagcatgaggatcac

SEQ ID NO: 80 - DNA, 21 bp, Artificial sequence
  Single strand DNA oligonucleotide
gaagggtcgaatcaaacataa

SEQ ID NO: 81 - DNA, 19 bp, Artificial sequence
  Single strand DNA oligonucleotide
gaggcctgtcccgtttgcg

SEQ ID NO: 82 - DNA, 21 bp, Artificial sequence
  Single strand DNA oligonucleotide
caatgggaaatttcaaatatg

SEQ ID NO: 83 - DNA, 21 bp, Artificial sequence
  Single strand DNA oligonucleotide
gttccagaaaacccagagatg

SEQ ID NO: 84 - DNA, 21 bp, Artificial sequence
  Single strand DNA oligonucleotide
gtttctgctgattctccctgg

SEQ ID NO: 85 - DNA, 20 bp, Artificial sequence
  Single strand DNA oligonucleotide
ccgatgccgtgtcaatctcc

SEQ ID NO: 86 - DNA, 28 bp, Artificial sequence
  Single strand DNA oligonucleotide
ttgagctccaatttgaaactgctggtaa

SEQ ID NO: 87 - DNA, 20 bp, Artificial sequence
  Single strand DNA oligonucleotide
ccgatgccgtgtcaatctcc

SEQ ID NO: 88 - DNA, 22 bp, Artificial sequence
  Single strand DNA oligonucleotide
gttttcttcgggtaactgagtg

SEQ ID NO: 89 - DNA, 19 bp, Artificial sequence
  Single strand DNA oligonucleotide
caaccgaacggaagaagtg

SEQ ID NO: 90 - DNA, 20 bp, Artificial sequence
  Single strand DNA oligonucleotide
gattaaggacatccagtatg

SEQ ID NO: 91 - DNA, 59 bp, Artificial sequence
  Single strand DNA oligonucleotide
cttattttgtaattacataactgagacagtagagaatggaggcgccggtgcctcctccg

SEQ ID NO: 92 - DNA, 8149 bp, Artificial sequence
  pMS2CPGFP(x3)
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccggatgcaagggttcgaatcccttagctctcattattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatactctagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggatcccccgggctgcaggaattcgatatcgtgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaagatatcgtgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaagagatcaagcttatcgataccgtcgacctcgacatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactggtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcgccagtagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacgggaatttatggtgcactctcagtacaatctgctctgatgccgcatagttaagccagccccgacacccgccaacacgcgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 93 - DNA, 62 bp, Artificial sequence
  Single strand DNA oligonucleotide
gcagactggaaaagatgtaatgaaaggtgcccttggttttggaggcgccggtgcctcctccg

SEQ ID NO: 94 - DNA, 21 bp, Artificial sequence
  Stem and loop MS2L
aaacatgaggatcacccatgt

SEQ ID NO: 95 - DNA, 62 bp, Artificial sequence
  Single strand DNA oligonucleotide
caaaattgttcacaaatcaaacttcattaataacaaaaaaggaggcgccggtgcctcctccg

SEQ ID NO: 96 - DNA, 5242 bp, Artificial sequence
  pRFPLOXHIS5MS2L
gaacgcggccgccagctgaagcttaggcgccggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcgaagcttcgtacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaggagggcttttgtagaaagaaatacgaacgaaacgaaaatcagcgttgccatcgctttggacaaagctcccttacctgaagagtcgaattttattgatgaacttataacttccaagcatgcaaaccaaaagggagaacaagtaatccaagtagacacgggaattggattcttggatcacatgtatcatgcactggctaaacatgcaggctggagcttacgactttactcaagaggtgatttaatcatcgatgatcatcacactgcagaagatactgctattgcacttggtattgcattcaagcaggctatgggtaactttgccggcgttaaaagatttggacatgcttattgtccacttgacgaagctctttctagaagcgtagttgacttgtcgggacggccctatgctgttatcgatttgggattaaagcgtgaaaaggttggggaattgtcctgtgaaatgatccctcacttactatattccttttcggtagcagctggaattactttgcatgttacctgcttatatggtagtaatgaccatcatcgtgctgaaagcgcttttaaatctctggctgttgccatgcgcgcggctactagtcttactggaagttctgaagtcccaagcacgaagggagtgttgtaaagagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtttaaacgagctctcgagaacccttaatataacttcgtataatgtatgctatacgaagttattaggtgatatcaacccgggccctatatatggatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatctgcgcgcgatcgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 97 - DNA, 62 bp, Artificial sequence
  Single strand DNA oligonucleotide
cagcaactttggcgtctccagctcgttttccttcaagcctggaggcgccggtgcctcctccg

SEQ ID NO: 98 - DNA, 34 bp, Artificial sequence
  loxP site
ataacttcgtataatgtatgctatacgaagttat

SEQ ID NO: 99 - DNA, 714 bp, Artificial sequence
  GFP coding sequence
gtgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaa

SEQ ID NO: 100 - DNA, 698 bp, Artificial sequence
  RFP coding sequence
agcttaggcgccggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcga

SEQ ID NO: 101 - DNA, 698 bp, Artificial sequence
  MS2L sequence
atcaacccgggccctatatatggatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatctgcgcgcgatcgatatca

SEQ ID NO: 102 - DNA, 426 bp, Artificial sequence
  CP-MS2
tctagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggatcccc

SEQ ID NO: 103 - DNA, 4544 bp, Artificial sequence
  pLOXPHIS5MS2L
gaacgcggccgccagctgaagcttcgtacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaggagggcttttgtagaaagaaatacgaacgaaacgaaaatcagcgttgccatcgctttggacaaagctcccttacctgaagagtcgaattttattgatgaacttataacttccaagcatgcaaaccaaaagggagaacaagtaatccaagtagacacgggaattggattcttggatcacatgtatcatgcactggctaaacatgcaggctggagcttacgactttactcaagaggtgatttaatcatcgatgatcatcacactgcagaagatactgctattgcacttggtattgcattcaagcaggctatgggtaactttgccggcgttaaaagatttggacatgcttattgtccacttgacgaagctctttctagaagcgtagttgacttgtcgggacggccctatgctgttatcgatttgggattaaagcgtgaaaaggttggggaattgtcctgtgaaatgatccctcacttactatattccttttcggtagcagctggaattactttgcatgttacctgcttatatggtagtaatgaccatcatcgtgctgaaagcgcttttaaatctctggctgttgccatgcgcgcggctactagtcttactggaagttctgaagtcccaagcacgaagggagtgttgtaaagagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtttaaacgagctctcgagaacccttaatataacttcgtataatgtatgctatacgaagttattaggtgatatcaacccgggccctatatatggatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatctgcgcgcgatcgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 104 - protein, 130 aa, Artificial sequence
  Enterobacteria phage MS2 coat protein amino acid sequence
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 105 - DNA, 4548 bp, Artificial sequence
  pRFPLOXHIS5
gaacgcggccgccagctgaagcttaggcgccggtgcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcgaagcttcgtacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaggagggcttttgtagaaagaaatacgaacgaaacgaaaatcagcgttgccatcgctttggacaaagctcccttacctgaagagtcgaattttattgatgaacttataacttccaagcatgcaaaccaaaagggagaacaagtaatccaagtagacacgggaattggattcttggatcacatgtatcatgcactggctaaacatgcaggctggagcttacgactttactcaagaggtgatttaatcatcgatgatcatcacactgcagaagatactgctattgcacttggtattgcattcaagcaggctatgggtaactttgccggcgttaaaagatttggacatgcttattgtccacttgacgaagctctttctagaagcgtagttgacttgtcgggacggccctatgctgttatcgatttgggattaaagcgtgaaaaggttggggaattgtcctgtgaaatgatccctcacttactatattccttttcggtagcagctggaattactttgcatgttacctgcttatatggtagtaatgaccatcatcgtgctgaaagcgcttttaaatctctggctgttgccatgcgcgcggctactagtcttactggaagttctgaagtcccaagcacgaagggagtgttgtaaagagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtttaaacgagctctcgagaacccttaatataacttcgtataatgtatgctatacgaagttattaggtgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 106 - DNA, 60 bp, Artificial sequence
  Single strand DNA oligonucleotide
gaaaattgagcatgttgatgatgaaacgcgtacacactagacgctgcaggtcgacaaccc

SEQ ID NO: 107 - DNA, 60 bp, Artificial sequence
  Single strand DNA oligonucleotide
tatatatatatgcgaatatatgtgtgcaaatattgatgcagcataggccactagtggatc

SEQ ID NO: 108 - DNA, 60 bp, Artificial sequence
  Single strand DNA oligonucleotide
atctatccttggtatgcaagacatgtggaaagctacatagacgctgcaggtcgacaaccc

SEQ ID NO: 109 - DNA, 70 bp, Artificial sequence
  Single strand DNA oligonucleotide
tcaaacataagcggagaatagccaaataaaaaaaaaagatgaaaagaaaggcataggccactagtggatc

SEQ ID NO: 110 - DNA, 60 bp, Artificial sequence
  Single strand DNA oligonucleotide
tgaagtggtgcgcttctatactattgaagctaaattgtaaacgctgcaggtcgacaaccc

SEQ ID NO: 111 - DNA, 60 bp, Artificial sequence
  Single strand DNA oligonucleotide
atgaagagtgtaataggtaagtataagtattatttaatcagcataggccactagtggatc

SEQ ID NO: 112 - DNA, 60 bp, Artificial sequence
  Single strand DNA oligonucleotide
gccggacatgattgaagaattagatctacatgaagattagacgctgcaggtcgacaaccc

SEQ ID NO: 113 - DNA, 60 bp, Artificial sequence
  Single strand DNA oligonucleotide
agtgggggaaagtatgatatgttatctttctccaataaatgcataggccactagtggatc

SEQ ID NO: 114 - DNA, 60 bp, Artificial sequence
  Single strand DNA oligonucleotide
cctaaagcacaacggacaacgcaagctggcttccacttgaacgctgcaggtcgacaaccc

SEQ ID NO: 115 - DNA, 60 bp, Artificial sequence
  Single strand DNA oligonucleotide
tctaaacgcaatgtgcttatttcagtaatagtaaggattcgcataggccactagtggatc

SEQ ID NO: 116 - DNA, 60 bp, Artificial sequence
  Single strand DNA oligonucleotide
cgccttggaaaatgagaataaacgaaaagcaaagttatgaacgctgcaggtcgacaaccc

SEQ ID NO: 117 - DNA, 60 bp, Artificial sequence
  Single strand DNA oligonucleotide
aaaatattcacaaattaattgaagaggaaaggtgaaaaatgcataggccactagtggatc

SEQ ID NO: 118 - DNA, 60 bp, Artificial sequence
  Single strand DNA oligonucleotide
atacaaggaattggtcaaaaacattgaaagcaaactatagacgctgcaggtcgacaaccc

SEQ ID NO: 119 - DNA, 60 bp, Artificial sequence
  Single strand DNA oligonucleotide
aaaaatatgcagaggggtgtaaaagtaggatgtaatccaagcataggccactagtggatc

SEQ ID NO: 120 - DNA, 60 bp, Artificial sequence
  Single strand DNA oligonucleotide
aaaacatgcttctgagctttcgagtaactccaaattttgaacgctgcaggtcgacaaccc

SEQ ID NO: 121 - DNA, 60 bp, Artificial sequence
  Single strand DNA oligonucleotide
tgtcgtggaaacaacgccactcatttgttacttgagcgttgcataggccactagtggatc

SEQ ID NO: 122 - DNA, 60 bp, Artificial sequence
  Single strand DNA oligonucleotide
taggcagctgggctcgaaacaaaggaagcatcgtttatgaacgctgcaggtcgacaaccc

SEQ ID NO: 123 - DNA, 60 bp, Artificial sequence
  Single strand DNA oligonucleotide
tatattgtgtgtgcgttttgtttcactgagaaagcggacggcataggccactagtggatc

SEQ ID NO: 124 - DNA, 60 bp, Artificial sequence
  Single strand DNA oligonucleotide
atacgccgaaggttcactagtcaagacagaaaagctttagacgctgcaggtcgacaaccc

SEQ ID NO: 125 - DNA, 60 bp, Artificial sequence
  Single strand DNA oligonucleotide
ttttttctagtttgaatgtgttccaaatcgtcataagtacgcataggccactagtggatc

SEQ ID NO: 126 - DNA, 60 bp, Artificial sequence
  Single strand DNA oligonucleotide
tgattgggaagccatcgatgcacaaacaattaaattatgaacgctgcaggtcgacaaccc

SEQ ID NO: 127 - DNA, 60 bp, Artificial sequence
  Single strand DNA oligonucleotide
tgcaaggaaaaatactttatcctaattcaggaacatcaaagcataggccactagtggatc

SEQ ID NO: 128 - DNA, 60 bp, Artificial sequence
  Single strand DNA oligonucleotide
atttcagaggagatccatatctggtcttggcgacctttgaacgctgcaggtcgacaaccc

SEQ ID NO: 129 - DNA, 60 bp, Artificial sequence
  Single strand DNA oligonucleotide
acacctacattcatttgtgcagttatgctttgaacttcatgcataggccactagtggatc

SEQ ID NO: 130 - DNA, 60 bp, Artificial sequence
  Single strand DNA oligonucleotide
tttgtatgaattaaaaggattactaggaaatgattcatgaacgctgcaggtcgacaaccc

SEQ ID NO: 131 - DNA, 60 bp, Artificial sequence
  Single strand DNA oligonucleotide
gtgtaattagttatttcaaagtacatattaaaatatattagcataggccactagtggatc

SEQ ID NO: 132 - DNA, 60 bp, Artificial sequence
  Single strand DNA oligonucleotide
aaaaggcaagagtttcatcctagactcttccaagctatgaacgctgcaggtcgacaaccc

SEQ ID NO: 133 - DNA, 60 bp, Artificial sequence
  Single strand DNA oligonucleotide
aggagtatagagttaagaaaaatataaaaattgaagtagcgcataggccactagtggatc

SEQ ID NO: 134 - DNA, 60 bp, Artificial sequence
  Single strand DNA oligonucleotide
ctataaaaacttacgtaagacctcatcgagccatctatagacgctgcaggtcgacaaccc

SEQ ID NO: 135 - DNA, 60 bp, Artificial sequence
  Single strand DNA oligonucleotide
ctgaagcacgcctatttatcaatgtttattatattaaaaagcataggccactagtggatc

SEQ ID NO: 136 - DNA, 60 bp, Artificial sequence
  Single strand DNA oligonucleotide
ctacatgaagcacctgctggagtgccgctcgctttggtaaacgctgcaggtcgacaaccc

SEQ ID NO: 137 - DNA, 60 bp, Artificial sequence
  Single strand DNA oligonucleotide
tgagagtattgttaggcaacgcattataccacagttttttgcataggccactagtggatc

SEQ ID NO: 138 - DNA, 60 bp, Artificial sequence
  Single strand DNA oligonucleotide
tggatcctctgggagactgaccgcctcaccagtgtactaaacgctgcaggtcgacaaccc

SEQ ID NO: 139 - DNA, 60 bp, Artificial sequence
  Single strand DNA oligonucleotide
atacacatatatagagatacaagcgagggaacggggccctgcataggccactagtggatc

SEQ ID NO: 140 - DNA, 60 bp, Artificial sequence
  Single strand DNA oligonucleotide
gtacttcctagcagaaagagagcggatcaacaaccattgaacgctgcaggtcgacaaccc

SEQ ID NO: 141 - DNA, 60 bp, Artificial sequence
  Single strand DNA oligonucleotide
cccattgtttgccattcgaacacatccatcctacgtggtagcataggccactagtggatc

SEQ ID NO: 142 - DNA, 60 bp, Artificial sequence
  Single strand DNA oligonucleotide
tcattatgaagcggtgagagctaattttgaaggtgcttaaacgctgcaggtcgacaaccc

SEQ ID NO: 143 - DNA, 60 bp, Artificial sequence
  Single strand DNA oligonucleotide
atatttacaaatttacctatacgctctgagttgatattacgcataggccactagtggatc

SEQ ID NO: 144 - DNA, 60 bp, Artificial sequence
  Single strand DNA oligonucleotide
atgggatggaaatttatttgtatggaacggcttaggttgaacgctgcaggtcgacaaccc

SEQ ID NO: 145 - DNA, 60 bp, Artificial sequence
  Single strand DNA oligonucleotide
gtttaaataatgcaaaaaatttgtgtaaaaagaatatgtggcataggccactagtggatc

SEQ ID NO: 146 - DNA, 60 bp, Artificial sequence
  Single strand DNA oligonucleotide
gtacacaacggtcttatcaagtcaatcttctaaattatagacgctgcaggtcgacaaccc

SEQ ID NO: 147 - DNA, 60 bp, Artificial sequence
  Single strand DNA oligonucleotide
aggaatataaaaaggcgctactataaagtacttaatgatagcataggccactagtggatc

SEQ ID NO: 148 - DNA, 60 bp, Artificial sequence
  Single strand DNA oligonucleotide
acactgtcaaccacaggaaattctggtcctgcggcaatagacgctgcaggtcgacaaccc

SEQ ID NO: 149 - DNA, 60 bp, Artificial sequence
  Single strand DNA oligonucleotide
gacaatgctaaaagagtagtcaaattattgattagttcctgcataggccactagtggatc

SEQ ID NO: 150 - DNA, 60 bp, Artificial sequence
  Single strand DNA oligonucleotide
atgggaagttgtgacaggtattaggaagctactaatctgaacgctgcaggtcgacaaccc

SEQ ID NO: 151 - DNA, 60 bp, Artificial sequence
  Single strand DNA oligonucleotide
tatatattacacagaattattttcttcacttcctccgtcagcataggccactagtggatc

SEQ ID NO: 152 - DNA, 60 bp, Artificial sequence
  Single strand DNA oligonucleotide
aatcaaaggttggtttgtgaatggccaagtgccaaggtaaacgctgcaggtcgacaaccc

SEQ ID NO: 153 - DNA, 60 bp, Artificial sequence
  Single strand DNA oligonucleotide
cactagagcgttttaaattcaatgctattatttttgattggcataggccactagtggatc

SEQ ID NO: 154 - DNA, 60 bp, Artificial sequence
  Single strand DNA oligonucleotide
cgatgtagaggatgtgctgattgacactttatgcaattaaacgctgcaggtcgacaaccc

SEQ ID NO: 155 - DNA, 60 bp, Artificial sequence
  Single strand DNA oligonucleotide
tttattctttacatactgttacaagaaactcttttctacagcataggccactagtggatc

SEQ ID NO: 156 - DNA, 60 bp, Artificial sequence
  Single strand DNA oligonucleotide
gataacaacaaagaggtcactttgctcttcaaaagattgaacgctgcaggtcgacaaccc

SEQ ID NO: 157 - DNA, 60 bp, Artificial sequence
  Single strand DNA oligonucleotide
tatatatgtacatatctatatgtatacatatttttatatagcataggccactagtggatc

SEQ ID NO: 158 - DNA, 60 bp, Artificial sequence
  Single strand DNA oligonucleotide
caaagtcacttcggctaatgaacatacaagcgctgtttgaacgctgcaggtcgacaaccc

SEQ ID NO: 159 - DNA, 60 bp, Artificial sequence
  Single strand DNA oligonucleotide
acgaaataaagagggatgcaacgaacttggtcatctgttggcataggccactagtggatc

SEQ ID NO: 160 - DNA, 60 bp, Artificial sequence
  Single strand DNA oligonucleotide
aatcgaagagctaacagacactctcaattcaactatataaacgctgcaggtcgacaaccc

SEQ ID NO: 161 - DNA, 60 bp, Artificial sequence
  Single strand DNA oligonucleotide
gactgtatcatcagtgaacatatagtataacaaatcaagtgcataggccactagtggatc

SEQ ID NO: 162 - DNA, 60 bp, Artificial sequence
  Single strand DNA oligonucleotide
aaatccaaccattggtcgcgatagcaagaaggccgtatgaacgctgcaggtcgacaaccc

SEQ ID NO: 163 - DNA, 60 bp, Artificial sequence
  Single strand DNA oligonucleotide
tagagattatattatgtaaaggtaaaaacgggagcgagcagcataggccactagtggatc

SEQ ID NO: 164 - DNA, 60 bp, Artificial sequence
  Single strand DNA oligonucleotide
aatacaaatatctgatgtttcaatgtctccttctctataaacgctgcaggtcgacaaccc

SEQ ID NO: 165 - DNA, 60 bp, Artificial sequence
  Single strand DNA oligonucleotide
accagtgtgaacgttgttgtccatatggggcatgcactcagcataggccactagtggatc

SEQ ID NO: 166 - DNA, 60 bp, Artificial sequence
  Single strand DNA oligonucleotide
caggtcaagaaaatggaaacgacgcctcttccatttgtaaacgctgcaggtcgacaaccc

SEQ ID NO: 167 - DNA, 60 bp, Artificial sequence
  Single strand DNA oligonucleotide
accaactatatatgcagtttagaggcttaaagcaatactagcataggccactagtggatc

SEQ ID NO: 168 - DNA, 60 bp, Artificial sequence
  Single strand DNA oligonucleotide
tgagaggacgaagctacgggaaaagcttgaaattatttgaacgctgcaggtcgacaaccc

SEQ ID NO: 169 - DNA, 60 bp, Artificial sequence
  Single strand DNA oligonucleotide
tatattcgctaaataaaatctctccctttctagggtgtttgcataggccactagtggatc

SEQ ID NO: 170 - DNA, 60 bp, Artificial sequence
  Single strand DNA oligonucleotide
aaaagttaaaacaaaaaaggaagaaggaaaggagaggtaaacgctgcaggtcgacaaccc

SEQ ID NO: 171 - DNA, 60 bp, Artificial sequence
  Single strand DNA oligonucleotide
caatttatacatgatttggatcctcctttggctatgtatggcataggccactagtggatc

SEQ ID NO: 172 - DNA, 20 bp, Artificial sequence
  Single strand DNA oligonucleotide
tagcacctggcgtattgact

SEQ ID NO: 173 - DNA, 20 bp, Artificial sequence
  Single strand DNA oligonucleotide
cgaggaaggaaatagtaacg

SEQ ID NO: 174 - DNA, 20 bp, Artificial sequence
  Single strand DNA oligonucleotide
gcctcaatatttgcctggac

SEQ ID NO: 175 - DNA, 20 bp, Artificial sequence
  Single strand DNA oligonucleotide
tggatctctcctgcctaatc

SEQ ID NO: 176 - DNA, 24 bp, Artificial sequence
  Single strand DNA oligonucleotide
caattactaggtcgtctgttggtc

SEQ ID NO: 177 - DNA, 20 bp, Artificial sequence
  Single strand DNA oligonucleotide
ccacattggtgtatagttgg

SEQ ID NO: 178 - DNA, 20 bp, Artificial sequence
  Single strand DNA oligonucleotide
gtccactcaaacttcagacg

SEQ ID NO: 179 - DNA, 20 bp, Artificial sequence
  Single strand DNA oligonucleotide
gtttctgcactttcacttgc

SEQ ID NO: 180 - DNA, 20 bp, Artificial sequence
  Single strand DNA oligonucleotide
attcgctgggttagaatacc

SEQ ID NO: 181 - DNA, 20 bp, Artificial sequence
  Single strand DNA oligonucleotide
cgtcgcttcccacatcgtcc

SEQ ID NO: 182 - DNA, 20 bp, Artificial sequence
  Single strand DNA oligonucleotide
aacaagccattggggatgcc

SEQ ID NO: 183 - DNA, 20 bp, Artificial sequence
  Single strand DNA oligonucleotide
ccctggcattgttagacatc

SEQ ID NO: 184 - DNA, 20 bp, Artificial sequence
  Single strand DNA oligonucleotide
tgttaataacgacgcagagg

SEQ ID NO: 185 - DNA, 20 bp, Artificial sequence
  Single strand DNA oligonucleotide
ggaatgactaccgccacccc

SEQ ID NO: 186 - DNA, 20 bp, Artificial sequence
  Single strand DNA oligonucleotide
cagtggtaatgcaataaagg

SEQ ID NO: 187 - DNA, 20 bp, Artificial sequence
  Single strand DNA oligonucleotide
tcaagtggaagcggagtggg

SEQ ID NO: 188 - DNA, 20 bp, Artificial sequence
  Single strand DNA oligonucleotide
aaaccttggtgaggaggaag

SEQ ID NO: 189 - DNA, 20 bp, Artificial sequence
  Single strand DNA oligonucleotide
tagtaccagcagcgggaagg

SEQ ID NO: 190 - DNA, 20 bp, Artificial sequence
  Single strand DNA oligonucleotide
cacgaatggtttaaccgctg

SEQ ID NO: 191 - DNA, 18 bp, Artificial sequence
  Single strand DNA oligonucleotide
gaatactttcccatccgc

SEQ ID NO: 192 - DNA, 20 bp, Artificial sequence
  Single strand DNA oligonucleotide
gaagggtgatgaccacattc

SEQ ID NO: 193 - DNA, 20 bp, Artificial sequence
  Single strand DNA oligonucleotide
tctatttggattgttccctc

SEQ ID NO: 194 - DNA, 20 bp, Artificial sequence
  Single strand DNA oligonucleotide
tctctgctcagatgcaatgc

SEQ ID NO: 195 - DNA, 20 bp, Artificial sequence
  Single strand DNA oligonucleotide
atactatgagccggggaggg

SEQ ID NO: 196 - DNA, 20 bp, Artificial sequence
  Single strand DNA oligonucleotide
atgcgcacgggctggcaatc

SEQ ID NO: 197 - DNA, 19 bp, Artificial sequence
  Single strand DNA oligonucleotide
gtcagaagcgttgttaccc

SEQ ID NO: 198 - DNA, 20 bp, Artificial sequence
  Single strand DNA oligonucleotide
gggactctttgcacgagacg

SEQ ID NO: 199 - DNA, 20 bp, Artificial sequence
  Single strand DNA oligonucleotide
ttgaaggggggtatctttgg

SEQ ID NO: 200 - DNA, 20 bp, Artificial sequence
  Single strand DNA oligonucleotide
ggtggtgaaaagcaaagagt

SEQ ID NO: 201 - DNA, 20 bp, Artificial sequence
  Single strand DNA oligonucleotide
aggttgtccttgatacgtgg

SEQ ID NO: 202 - DNA, 20 bp, Artificial sequence
  Single strand DNA oligonucleotide
gatcaacaggtgccactttg

SEQ ID NO: 203 - DNA, 20 bp, Artificial sequence
  Single strand DNA oligonucleotide
cagtgggtcgtgacatgaat